(19) United States
(12) Patent Application Publication
(10) Pub. No.: US 2010/0063065 A1
(43) Pub. Date: Mar. 11, 2010

(54) THIOPHENE DERIVATIVES AS PPAR AGONISTS I

(75) Inventors: Andrew Ayscough, Ely (GB); David Rodyney Owen, Cambridge (GB); Paul Meo, Cambridge (GB); David James Pearson, Hertfordshire (GB); Yvonne Walker, Cambridge (GB); Richard Justin Boyce, Cambridge (GB); Fabio Zuccotto, Hitchin (GB)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Inpharmatica Limited, London (GB)

(21) Appl. No.: 12/088,333

(22) PCT Filed: Sep. 28, 2006
(86) PCT No.: PCT/GB2006/003620

§ 371 Date: Mar. 27, 2008

(30) Foreign Application Priority Data
Sep. 29, 2005 (GB) 0519873.4
Jul. 21, 2006 (GB) 0614580.9

(51) Int. Cl. A61K 31/381; C07D 333/36; C07D 409/10; A61K 31/506; A61K 31/4436; A61P 3/10; A61P 3/04; A61P 17/02; A61P 29/00; A61P 25/00

(52) U.S. Cl. 514/256; 549/68; 549/69; 544/333; 546/280.4; 514/447; 514/336

(57) ABSTRACT

The invention discloses compounds of formula (I); wherein: R is a carboxylic acid or a derivative thereof; R¹ is alkyl, alkenyl, alkynyl, cycloalkyl, alkoxy, alkylthio, halo or trihalomethyl; R² is aryl, heteroaryl, arylalkyl or heteroarylalkyl; R³ is H or F; and L is a linking group comprising a chain of from 2 to 8 atoms linking R and the carbonyl group (A); and pharmaceutically acceptable derivatives thereof, useful for treating disorders mediated by peroxisome-proliferator-activated receptor (PPAR) subtype δ (PPARδ). The compounds of the invention are therefore useful in the treatment of metabolic syndrome, obesity, type-II diabetes, dyslipidemia, wound healing, inflammation, neurodegenerative disorders and multiple sclerosis.

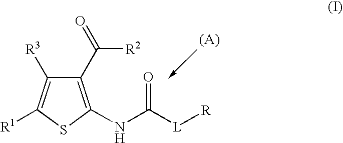

All documents cited herein are incorporated by reference in their entirety.

### TECHNICAL FIELD

This invention relates to thienyl compounds which are useful for treating disorders mediated by peroxisome-proliferator-activated receptor (PPAR) subtype δ (PPARδ).

### BACKGROUND OF THE INVENTION

The high fat diet of modern society combined with a largely sedentary lifestyle has resulted in an increase in the population that are overweight or obese. Being overweight or obese increases the risk of coronary heart disease, hypertension, dyslipidemia, atherosclerosis, type-II diabetes, stroke, osteoarthritis, restrictive pulmonary disease, sleep apnoea, certain types of cancers and inflammatory disorders. The standard treatment for obesity is calorific restriction and increase of physical exercise. However, such approaches are rarely successful and pharmaceutical treatments are required to correct these metabolic disorders.

The three peroxisome-proliferator-activated receptor (PPAR) subtypes, PPARγ, PPARα and PPARδ, are nuclear receptors that regulate glucose and lipid homeostasis.

Pharmacological evidence gained with small molecule agonists and genetic studies has uncovered several important roles of PPARδ in regulating lipid metabolism and energy homeostasis (1). The data indicate that PPARδ agonists might be useful in the treatment of various components of the metabolic syndrome including dyslipidemia, obesity and insulin resistance by increasing fatty acid consumption in skeletal muscle and adipose tissue.

PPARδ agonists have shown cholesterol lowering activity and elevation of high-density lipoprotein cholesterol (HDL-C) levels in diabetic mice suggesting they may have beneficial effects on dyslipidemia (2). A potent PPARδ agonist has also been shown to increase HDL-C while decreasing elevated triglyceride (TG) and insulin levels in obese rhesus monkeys (3). The same compound also attenuates weight gain and insulin resistance in mice fed high-fat diets by increasing the expression of genes in skeletal muscle that promote lipid catabolism and mitochondrial uncoupling, thereby increasing β-oxidation of fatty acids in skeletal muscle (4).

Genetic studies provide data that accord with that of the pharmacological experiments described above. Overexpression of constitutively active PPARδ in mouse adipose tissue protects against either genetic or high-fat-diet-induced hyperlipidemia, steatosis and obesity and increases the expression of genes that are involved in fatty acid oxidation and energy dissipation (5). Conversely, PPARδ null mice display an obese phenotype and reduced energy uncoupling when fed a high-fat diet. Recently, overexpression of constitutively active PPARδ in mouse skeletal muscle was found to induce differentiation of mitochondria-rich, oxidative type-1 muscle fibres (6). As a result, these transgenic animals are resistant to diet-induced obesity and their exercise endurance is improved.

Studies on PPARδ+/−mice show a delay in wound healing (7) and further animal model studies with a PPARδ agonist have demonstrated an enhancement in barrier repair and a reduction in inflammation (8).

A series of studies have demonstrated the expression of PPARδ in a number of neural cell types including optic nerve oligodendrocytes and sciatic nerve Schwann cells. A PPARδ agonist has demonstrated neuroprotective effects on cerebellar neurons suggesting a role in the treatment of neurodegenerative diseases including Alzheimer's disease and Parkinson's disease and may also be of use in the enhancement of learning and memory function (9). Studies with a PPARδ agonist show a reduction in the clinical signs of murine experimental autoimmune encephalomyelitis, commonly used as a model for multiple sclerosis (10).

Consequently, PPARδ agonists are expected to be therapeutically useful, e.g. in the treatment of metabolic syndrome, obesity, type-II diabetes, dyslipidemia, wound healing, inflammation, neurodegenerative disorders and multiple sclerosis. There is therefore a need for new and improved compounds which are PPARδ agonists.

### DISCLOSURE OF THE INVENTION

Compounds of formula (I) defined below, and pharmaceutically acceptable derivatives thereof, have been found to be agonists of PPARδ. Compounds of formula (I) or pharmaceutically acceptable derivatives thereof are thus useful in the treatment of conditions and diseases mediated by PPARδ, in particular metabolic syndrome, obesity, type-II diabetes, dyslipidemia, wound healing, inflammation, neurodegenerative disorders and multiple sclerosis.

The invention therefore provides a compound of formula (I):

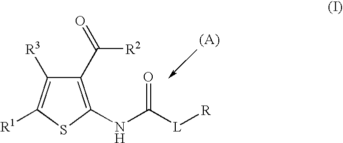

wherein:
R is a carboxylic acid or a derivative thereof;
R¹ is alkyl, alkenyl, alkynyl, cycloalkyl, alkoxy, alkylthio, halo or trihalomethyl;
R² is aryl, heteroaryl, arylalkyl or heteroarylalkyl;
R³ is H or F; and
L is a linking group comprising a chain of from 2 to 8 atoms linking R and the carbonyl group (A);
   and pharmaceutically acceptable derivatives thereof.

The invention also provides a compound of formula (I), or a pharmaceutically acceptable derivative thereof, for use in therapy. The invention further provides a pharmaceutical composition comprising a compound of formula (I), or a pharmaceutically acceptable derivative thereof, in combination with a pharmaceutically acceptable carrier, excipient or diluent.

The invention further provides a method for the treatment of a disease or condition mediated by PPARδ, comprising the step of administering a therapeutically effective amount of a compound of formula (I), or a pharmaceutically acceptable derivative thereof, to a patient. The invention also provides the use of a compound of formula (I), or a pharmaceutically acceptable derivative thereof, in the manufacture of a medicament for the treatment of a disease or condition mediated by PPARδ.

The invention also provides a composition comprising PPARδ and a compound of formula (I), or a pharmaceutically acceptable derivative thereof.

The invention also provides a crystal of PPARδ and a compound of formula (I), or a pharmaceutically acceptable derivative thereof. Such crystals can be used for X-ray diffraction studies of PPARδ inhibition, e.g. to provide atomic structural information in order to aid rational design of further agonists.

### Compounds of Formula (I) and Derivatives

The term “pharmaceutically acceptable derivative” includes any pharmaceutically acceptable salt, solvate or hydrate thereof.

The term “pharmaceutically acceptable salt” includes a salt prepared from pharmaceutically acceptable non-toxic acids or bases including inorganic or organic acids and bases.

Examples of inorganic acids suitable for use in this invention include, but are not limited to hydrochloric, hydrobromic, hydroiodic, sulfuric, and phosphoric acids. Appropriate organic acids for use in this invention include, but are not limited to aliphatic, aromatic, carboxylic and sulfonic classes of organic acids, examples of which include formic, acetic, propionic, citric, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic.

Examples of inorganic bases suitable for use in this invention include metallic salts made from aluminium, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

The compounds of the invention may exist in a number of diastereomeric and enantiomeric forms. Diastereomeric and enantiomeric forms of the polyphenols of the invention may be differentiated by the direction in which they rotate plane-polarised light. A dextrorotatory (d) substance rotates plane-polarised light in a clockwise or positive (+) direction. A levorotatory (l) substance rotates plane-polarised light in a counterclockwise or negative (−) direction. The invention encompasses pure diastereomers and enantiomers as well as mixtures, including racemic mixtures, of diastereomers and enantiomers.

R is a carboxylic acid or a derivative thereof. Derivatives of carboxylic acids include esters (e.g. of the formula —CO₂R⁴). R⁴ is alkyl (e.g. C₁₋₆alkyl) or arylalkyl (e.g. benzyl).

L is a linking group comprising a chain of 2 to 8 atoms linking R and the carbonyl group (A). The linking group L may therefore be any stable (i.e. not liable to decompose spontaneously) divalent linking group which separates R and the carbonyl group (A) by a chain of 2 to 8 atoms.

The chain may optionally be substituted by additional atoms or groups branching from the chain and/or the chain may optionally be substituted by additional atoms or groups forming cyclic moieties with the chain.

For example, L may be a chain of carbon atoms substituted by hydrogen (e.g. —(CH₂)₆—) or other groups (e.g. —CH₂CH(CH₃)CH₂—). Alternatively, where the chain is substituted by additional groups forming cyclic moieties with the chain, L includes structures such as

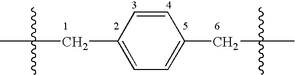

and the like. Where the chain length may be counted in more than one way, the chain length refers to the shortest chain length, e.g.

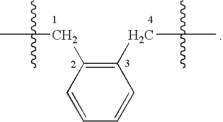

### Preferred Compounds

### Group R

Preferably, R is a carboxylic acid, i.e. —CO₂H.

### Group R¹

Preferably, R¹ is C₁₋₆alkyl, C₂₋₆alkenyl, C₂₋₆alkynyl, C₃₋₆cycloalkyl, C₁₋₆alkoxy, C₁₋₆alkylthio, halo (e.g. Cl) or trihalomethyl (e.g. CF₃). Especially preferred R¹ are C₁₋₆alkyl (more preferably methyl or ethyl) and Cl.

R¹ may be substituted or unsubstituted. Where substituted, R¹ may be substituted by one or more Sub¹, defined below. Preferred substituents on R¹ are halo, C₁₋₆alkylthio, C₁₋₆alkoxy, —S(O)R^{s} or —S(O)₂OR^{s}, where R^{s} is defined below.

### Group R²

Preferably, R² is aryl, heteroaryl, arylalkyl or heteroarylalkyl.

Particularly preferred R² are phenyl and pyridyl.

R² may be substituted or unsubstituted. Where substituted, R² may be substituted by one or more Sub¹, defined below. Preferred substituents on R² are OCF₃, CF₃, halo (e.g. F), aryl (e.g. phenyl), alkyl (e.g. C₁₋₆alkyl, such as methyl) and alkoxy (e.g. C₁₋₆alkoxy, such as methoxy). Particularly preferred substituents on R² are OCF₃ and halo (e.g. F).

Where R² is a phenyl group or a six-membered ring heteroaryl group (e.g. pyridyl) and is substituted, substitution at the meta and/or para positions is preferred, with para substitution being especially preferred.

### Group R³

Preferably, R³ is H.

### Group L

Preferably, the linking group L, in the orientation —(CO)-L-R, is -X-Y-Z-, where:
X is a single bond, alkylene, alkenylene, alkynylene, heteroalkylene, heteroalkenylene, heteroalkynylene, NR⁵, O, S, arylene, heteroarylene, cycloalkylene, heterocycloalkylene, cycloalkenylene or heterocycloalkenylene;
Y is a single bond, arylene, heteroarylene, cycloalkylene, heterocycloalkylene, cycloalkenylene or heterocycloalkenylene; and
Z is single bond, alkylene, alkenylene, alkynylene, heteroalkylene, heteroalkenylene, heteroalkynylene, NR⁵, O, S, arylene, heteroarylene, cycloalkylene, heterocycloalkylene, cycloalkenylene or heterocycloalkenylene;
provided that X, Y and Z are not each a single bond.

R⁵ is H, alkyl, aryl, —C(O)-alkyl, —C(O)-aryl, —S(O)₂-alkyl or —S(O)₂aryl.

More preferably,
X is a single bond, alkylene, alkenylene, alkynylene, heteroalkylene, heteroalkenylene, heteroalkynylene, NR⁵, O, or S;
Y is a single bond, arylene, heteroarylene, cycloalkylene, heterocycloalkylene, cycloalkenylene or heterocycloalkenylene; and
Z is single bond, alkylene, alkenylene, alkynylene, heteroalkylene, heteroalkenylene, heteroalkynylene, NR⁵, O, or S;
provided that X, Y and Z are not each a single bond.

Preferably, L comprises a chain of from 2 to 6 atoms linking R and the carbonyl group (A).

X may be unsubstituted or substituted. Where substituted, X may be substituted by one or more Sub¹, defined below. Preferred substituents on the group X are alkyl (e.g. C₁₋₆alkyl), alkoxy (e.g. C₁₋₆alkoxy), halogen, aryl (e.g. C₆₋₁₄aryl), heteroaryl (e.g. heteroaryl having 5-13 members), arylalkyl (e.g. C₆₋₁₄arylC₁₋₆alkyl) or heteroarylalkyl (e.g. heteroarylC₁₋₆alkyl, where heteroaryl has 5-13 members) or, alkylene where X is substituted by both ends of the alkylene (e.g. C₁₋₆alkylene) chain to form a cyclic group (e.g. cyclopentylene or cyclohexylene).

Y may be unsubstituted or substituted. Where substituted, Y may be substituted by one or more Sub¹, defined below.

Z may be unsubstituted or substituted. Where substituted, Z may be substituted by one or more Sub¹, defined below. Preferred substituents on the group Z are alkyl (e.g. C₁₋₆alkyl), alkoxy (e.g. C₁₋₆alkoxy), halogen, aryl (e.g. C₆₋₁₄aryl), heteroaryl (e.g. heteroaryl having 5-13 members), arylalkyl (e.g. C₆₋₁₄arylC₁₋₆alkyl) or heteroarylalkyl (e.g. heteroarylC₁₋₆alkyl, where heteroaryl has 5-13 members) or, alkylene where Z is substituted by both ends of the alkylene (e.g. C₁₋₆alkylene) chain to form a cyclic group (e.g. cyclopentylene or cyclohexylene).

X is preferably a single bond, alkylene, heteroalkylene, NR⁵ or O.

Y is preferably a single bond, arylene, heteroarylene, cycloalkylene or heterocycloalkylene.

Z is preferably a single bond, alkylene or heteroalkylene.

Preferred groups L, in the orientation —(CO)-L-R, are:
-(alkylene or heteroalkylene)-(arylene)-

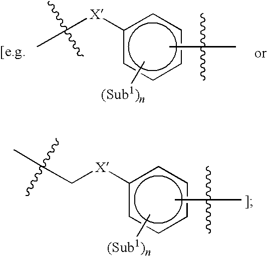

-(alkylene or heteroalkylene)-(arylene)-(alkylene or heteroalkylene)-

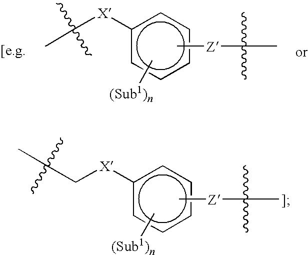

-(arylene)-(alkylene or heteroalkylene)-

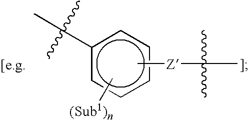

-(alkylene or heteroalkylene)- and

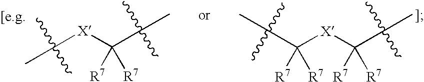

and
-(arylene)-

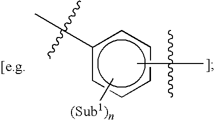

where:
X′ is CR⁷₂, O, S or NR⁶;
Sub¹ is defined below;
Z′ is (in the orientation —(CO)— . . . -Z′-R)—CR⁷CR⁷—, —O—CR⁷—, —S—CR⁷— or —NR⁶—CR⁷—;
R⁶ is H, alkyl, aryl, —C(O)-alkyl, —C(O)-aryl, —S(O)₂-alkyl or —S(O)₂-aryl, or R⁶, together with a Sub¹ or R⁷ group, is alkylene;
R⁷ is independently H or Sub¹, or two R⁷ are alkylene or heteroalkylene; and
n is 0, 1, 2 or 3.

R⁷ is preferably H.

R⁶ is preferably H or alkyl (e.g. C₁₋₆alkyl)

Preferred compounds of formula (I) are those of formula (II):

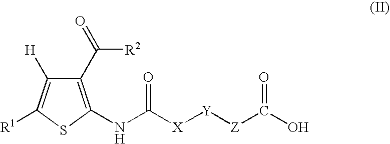

wherein R¹, R², X, Y and Z are defined above;
and pharmaceutically acceptable derivatives thereof.

Especially preferred compounds of the invention are the compounds of examples 1-103 below. Still more preferred compounds of the invention are the compounds of examples 1-5, 8-10, 12, 19, 22-24, 27-29, 31, 33, 34, 36-40, 43-45, 47, 54, 58, 59, 67, 71, 72, 75-77, 79-81, 83-87 and 92-103. Even more preferred compounds of the invention are the compounds of examples 1, 2, 22, 28, 29, 36, 38-40, 45, 67, 75-77, 79, 80, 83, 99 and 101.

Other preferred examples of the invention are the compounds of examples 120, 123, 131, 148, 161, 168, 174, 187, and 190. Even more preferred examples are the compounds of examples 112, 129, 146, 164, 179, 181, 182, 183, 184, 186, 188.

### Disclaimers

In some embodiments of the invention, e.g. the compounds of the invention, the compounds of formulae (IIIa)-(IIIg) are optionally disclaimed:

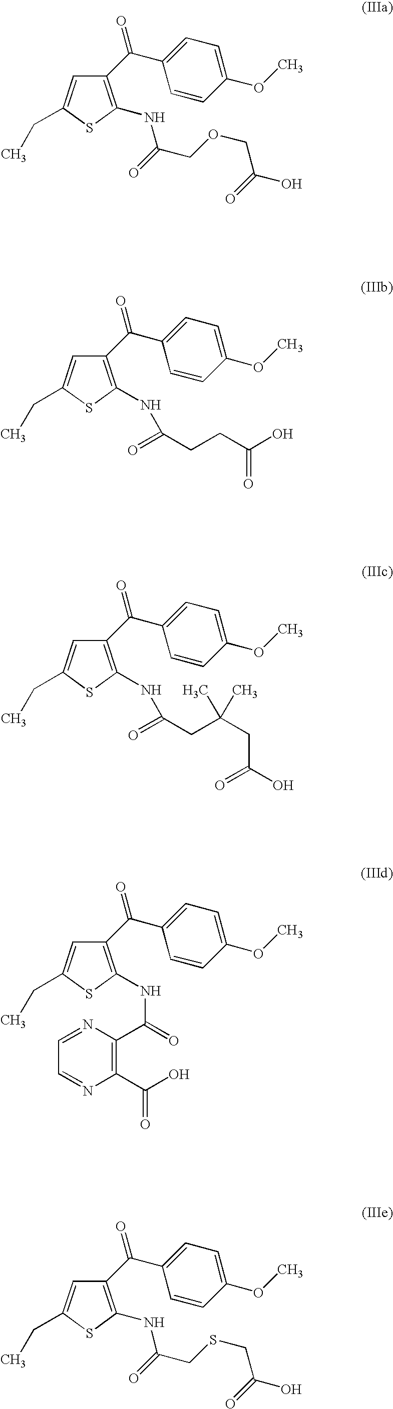

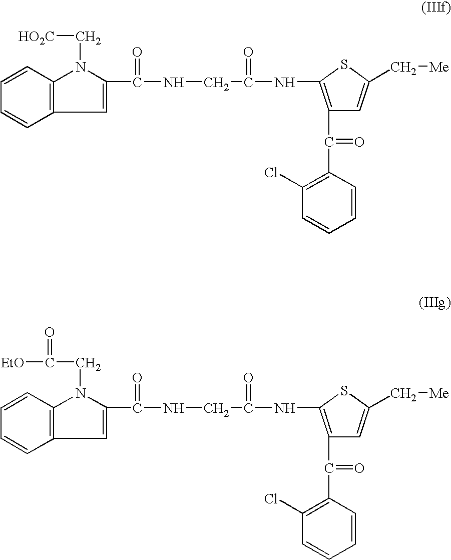

### Preparation

Methods for the preparation of the compounds of the invention are disclosed in detail below in the examples.

In general, compounds of the invention may be conveniently prepared by a general process wherein moiety A is coupled to an acid B using standard amide bond forming conditions. This synthesis is preferably carried out with the acid group protected by R′. Preferably, R′ is a C₁₋₆alkyl which can be hydrolysed after coupling of A and B to give a compound of formula (I) wherein R is a carboxylic acid.

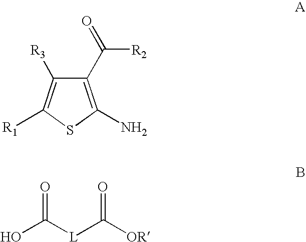

When L comprises a chain of 2 or 3 atoms linking R and the carbonyl group (A), it is preferable to react the moiety A with a cyclic anhydride C by heating the mixture in a high boiling point solvent such as toluene or acetonitrile to give compounds of formula (I) directly:

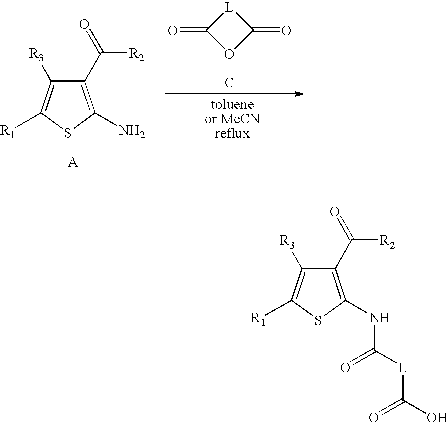

Alternatively, when X is alkylene, the synthesis can be carried out in a stepwise fashion wherein moiety A is coupled to a haloalkyl containing acid chloride D with a suitable non-nucleophilic base. The moiety E can then be coupled to moiety F by alkylation. The synthesis is carried out with the acid group protected by R′. L′ is a precursor of linker L which, together with CH₂ group a to the amide carbonyl of moiety E, forms the linker L when moiety E is reacted with moiety F:

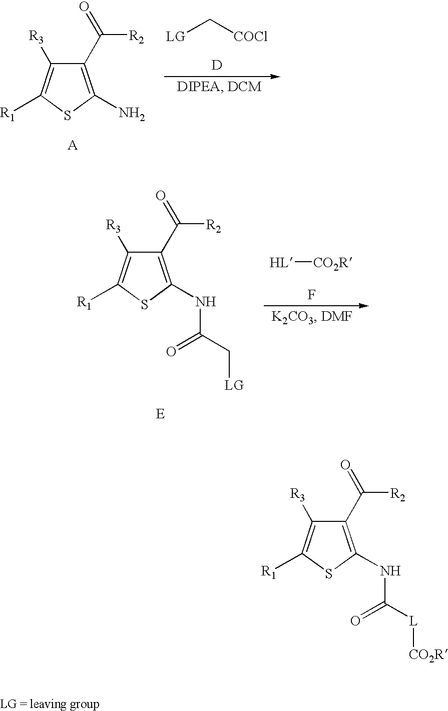

### Diseases and Conditions

Compounds of formula (I), and pharmaceutically acceptable derivatives thereof, have been found to be agonists of PPARδ.

Preferred compounds of the invention have an EC₅₀ in the PPARδ GAL4 assay of biological assay 1 of <1 μM, preferably <100 nM.

Preferred compounds of the invention up-regulate one or more of the target genes identified in biological assay 3 below (i.e. FATP, LCAD, CPT1, PDK4, UCP2, UCP3, PGC-1a and GLUT4) by at least 2 fold at sub-micromolar concentrations.

Preferred compounds of the invention demonstrate one or more of the following effects when compared to vehicle treated animals:
(i) improve lipid profiles through increasing HDL-cholesterol levels and/or reduce total cholesterol;
(ii) reduce triglyceride levels;
(iii) reduce glucose serum levels and improve oral glucose tolerance;
(iv) maintenance of body weight and/or promotion of lean tissue over fat mass from the results from the DEXA scanning and monitoring of body weight; and/or
(v) up-regulate one or more of the target genes identified in biological assay 3 below (i.e. FATP, LCAD, CPT1, PDK4, UCP2, UCP3, PGC-1a and GLUT4) by at least 2 fold at sub-micromolar concentrations.

Preferred compounds of the invention have an EC₅₀ in the PPARδ GAL4 assay of biological assay 1 at least ten times lower than its EC₅₀ in the PPARα GAL4 assay or the PPARγ GAL4 assay, preferably both, of biological assay 1.

The invention is useful for the treatment of a disease or condition mediated by PPARδ. Diseases and conditions mediated by PPARδ include: metabolic syndrome, and components thereof including dyslipidaemia, obesity and insulin resistance; type-II diabetes; wound healing; inflammation; neurodegenerative disorders; and multiple sclerosis. Since being overweight or obese increases certain risk factors, the present invention is useful for the treatment of coronary heart disease, hypertension, hyperlipidaemia, type-II diabetes mellitus, stroke, osteoarthritis, restrictive pulmonary disease, sleep apnoea and cancer.

As used herein, “treatment” includes prophylactic treatment. As used herein, a “patient” means an animal, preferably a mammal, preferably a human in need of treatment.

The amount of the compound of the invention administered should be a therapeutically effective amount where the compound or derivative is used for the treatment of a disease or condition and a prophylactically effective amount where the compound or derivative is used for the prevention of a disease or condition.

The term “therapeutically effective amount” used herein refers to the amount of compound needed to treat or ameliorate a targeted disease or condition. The term “prophylactically effective amount” used herein refers to the amount of compound needed to prevent a targeted disease or condition. The exact dosage will generally be dependent on the patient's status at the time of administration. Factors that may be taken into consideration when determining dosage include the severity of the disease state in the patient, the general health of the patient, the age, weight, gender, diet, time and frequency of administration, drug combinations, reaction sensitivities and the patient's tolerance or response to therapy. The precise amount can be determined by routine experimentation, but may ultimately lie with the judgement of the clinician. Generally, an effective dose will be from 0.01 mg/kg/day (mass of drug compared to mass of patient) to 50 mg/kg/day, preferably 0.05 mg/kg/day to 10 mg/kg/day. Compositions may be administered individually to a patient or may be administered in combination with other agents, drugs or hormones.

The compounds of the invention may be administered as a medicament by mucosal or parenteral routes, including intravenous, intramuscular, intraperitoneal, subcutaneous, transdermal, airway (aerosol), oral, intranasal, rectal, vaginal and topical (including buccal and sublingual) administration.

For parenteral administration, the compounds of the invention will generally be provided in injectable form. For oral administration, the compounds of the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Suitable binding agents include starch and gelatin. Suitable lubricating agents include magnesium stearate, stearic acid or talc. The tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients are mixed with water or an oil such as peanut oil, liquid paraffin or olive oil.

Compositions for use with the invention may comprise pharmaceutically acceptable carriers, such as sugars or salts, or excipients. They may also contain diluents, such as water, saline, glycerol, etc. Additionally, auxiliary substances, such as wetting or emulsifying agents, pH buffering substances, and the like, may be present. A thorough discussion of pharmaceutically acceptable carriers and excipients is available in Gennaro (2000) Remington: The Science and Practice of Pharmacy, 20th edition (ISBN: 0683306472).

### Chemical Groups

The term “halogen” (or “halo”) includes fluorine, chlorine, bromine and iodine.

Unless indicated explicitly otherwise, where combinations of groups are referred to herein as one moiety, e.g. arylalkyl, the last mentioned group contains the atom by which the moiety is attached to the rest of the molecule.

The terms “alkyl”, “alkylene”, “alkenyl”, “alkenylene”, “alkynyl”, or “alkynylene” are used herein to refer to both straight and branched chain acyclic forms. Cyclic analogues thereof are referred to as cycloalkyl, cycloalkylene, etc.

The term “alkyl” includes monovalent, straight or branched, saturated, acyclic hydrocarbyl groups. Preferred alkyl are C₁₋₁₀alkyl, more preferably C₁₋₆alkyl, still more preferably C₁₋₄alkyl, such as methyl, ethyl, n-propyl, i-propyl or t-butyl groups.

The term “cycloalkyl” includes monovalent, saturated, cyclic hydrocarbyl groups. Preferred cycloalkyl are C₃₋₆cycloalkyl, such as cyclopentyl and cyclohexyl.

The term “alkoxy” means alkyl-O—.

The term “alkylthio” means alkyl-S—.

The term “alkenyl” includes monovalent, straight or branched, unsaturated, acyclic hydrocarbyl groups having at least one carbon-carbon double bond and preferably no carbon-carbon triple bonds. Preferred alkenyl are C₂₋₁₀alkenyl, more preferably C₂₋₆alkenyl, still more preferably C₂₋₄alkenyl.

The term “cycloalkenyl” includes monovalent, unsaturated, cyclic hydrocarbyl groups having at least one carbon-carbon double bond and preferably no carbon-carbon triple bonds. Preferred cycloalkenyl are C₃₋₆cycloalkenyl, preferably C₅₋₆cycloalkenyl.

The term “alkynyl” includes monovalent, straight or branched, unsaturated, acyclic hydrocarbyl groups having at least one carbon-carbon triple bond and preferably no carbon-carbon double bonds. Preferred alkynyl are C₂₋₁₀alkynyl, more preferably C₂₋₆alkynyl, still more preferably C₂₋₄alkynyl.

The term “alkylene” includes divalent, straight or branched, saturated, acyclic hydrocarbyl groups. Preferred alkylene are C₁₋₁₀alkylene, more preferably C₁₋₆alkylene, still more preferably C₁₋₄alkylene, such as methylene, ethylene, n-propylene, i-propylene or t-butylene groups.

The term “cycloalkylene” includes divalent, saturated, cyclic hydrocarbyl groups. Preferred cycloalkylene are C₃₋₆cycloalkyl, such as cyclopentylene and cyclohexylene.

The term “alkenylene” includes divalent, straight or branched, unsaturated, acyclic hydrocarbyl groups having at least one carbon-carbon double bond and preferably no carbon-carbon triple bonds. Preferred alkenylene are C₁₋₁₀alkenylene, more preferably C₁₋₆alkenylene, still more preferably C₁₋₄alkenylene.

The term “cycloalkenylene” includes divalent, unsaturated, cyclic hydrocarbyl groups having at least one carbon-carbon double bond and preferably no carbon-carbon triple bonds. Preferred cycloalkenyl are C₃₋₆cycloalkenylene, preferably C₅₋₆cycloalkenylene.

The term “alkynylene” includes divalent, straight or branched, unsaturated, acyclic hydrocarbylene groups having at least one carbon-carbon triple bond and preferably no carbon-carbon double bonds. Preferred alkynylene are C₁₋₁₀alkynylene, more preferably C₁₋₆alkynylene, still more preferably C₁₋₄alkynylene.

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means allyl substituted with an aryl group, e.g. benzyl.

The term “arylene” includes divalent aromatic groups, such phenylene (e.g. phen-1,2-diyl, phen-1,3-diyl, or phen-1,4-diyl) or naphthylene (e.g. naphth-1,2-diyl, naphth-1,3-diyl, naphth-1,4-diyl, naphth-1,5-diyl, naphth-1,6-diyl, naphth-1,7-diyl, naphth-1,8-diyl, naphth-2,5-diyl, naphth-2,6-diyl, naphth-2,7-diyl or naphth-2,8-diyl). In general, the arylene groups may be monocyclic or polycyclic fused ring aromatic groups. Preferred arylene are C₆-C₁₄arylene.

Other examples of arylene groups are divalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “heteroaryl” includes monovalent, heteroaromatic, cyclic hydrocarbyl groups additionally containing one or more heteroatoms selected from O, S or N. In general, the heteroaryl groups may be monocyclic or polycyclic (e.g. bicyclic) fused ring heteroaromatic groups. Preferred heteroaryl groups are 5-13 membered (preferably 5-10 membered) and contain 1, 2, 3 or 4 heteroatoms selected from O, S or N.

Monocyclic heteroaromatic groups include 5- or 6-membered heteroaromatic groups containing 1, 2, 3 or 4 heteroatoms selected from O, S or N. Examples of monocyclic heteroaryl groups are pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-thiadiazolyl, pyridyl, pyrimidinyl, pyridazinyl, pyrazinyl, 1,3,5-triazinyl, 1,2,4-triazinyl, 1,2,3-triazinyl, tetrazolyl and succinimidyl.

Bicyclic heteroaromatic groups include 9- to 13-membered fused-ring heteroaromatic groups containing 1, 2, 3, 4 or more heteroatoms selected from O, S or N. Examples of bicyclic heteroaromatic groups are benzofuryl, [2,3-dihydro]benzofuryl, benzothienyl, benzotriazolyl, indolyl, isoindolyl, benzimidazolyl, imidazo[1,2-a]pyridyl, benzothiazolyl, benzoxazolyl, benzopyranyl, [3,4-dihydro]benzopyranyl, quinazolinyl, naphthyridinyl, pyrido[3,4-b]pyridyl, pyrido[3,2-b]pyridyl, pyrido[4,3-b]pyridyl, quinolinyl, isoquinolinyl, 5,6,7,8-tetrahydroquinolinyl, 5,6,7,8-tetrahydroisoquinolinyl and phthalimidyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The term “heteroarylalkyl” means alkyl substituted with an heteroaryl group.

The term “heteroarylene” includes divalent, heteroaromatic, cyclic hydrocarbyl groups additionally containing one or more heteroatoms selected from O, S or N. In general, the heteroaryl groups may be monocyclic or polycyclic (e.g. bicyclic) fused ring heteroaromatic groups. Preferred heteroaryl groups are 5-13 membered (preferably 5-10 membered) and contain 1, 2, 3 or 4 heteroatoms selected from O, S or N.

Monocyclic heteroaromatic groups include 5- or 6-membered heteroaromatic groups containing 1, 2, 3 or 4 heteroatoms selected from O, S or N. Examples of monocyclic heteroaryl groups are pyrrolylene, furylene, thienylene, imidazolylene, oxazolylene, isoxazolylene, thiazolylene, isothiazolylene, pyrazolylene, 1,2,3-triazolylene, 1,2,4-triazolylene, 1,2,3-oxadiazolylene, 1,2,4-oxadiazolylene, 1,2,5-oxadiazolylene, 1,3,4-oxadiazolylene, 1,3,4-thiadiazolylene, pyridylene, pyrimidinylene, pyridazinylene, pyrazinylene, 1,3,5-triazinylene, 1,2,4-triazinylene, 1,2,3-triazinylene, tetrazolylene and succinimidylene.

Bicyclic heteroaromatic groups include 9- to 13-membered fused-ring heteroaromatic groups containing 1, 2, 3, 4 or more heteroatoms selected from O, S or N. Examples of bicyclic heteroaromatic groups are benzofurylene, [2,3-dihydro]benzofurylene, benzothienylene, benzotriazolylene, indolylene, isoindolylene, benzimidazolylene, imidazo[1,2-a]pyridylene, benzothiazolylene, benzoxazolylene, benzopyranylene, [3,4-dihydro]benzopyranylene, quinazolinylene, naphthyridinylene, pyrido[3,4-b]pyridylene, pyrido[3,2-b]pyridylene, pyrido[4,3-b]pyridylene, quinolinylene, isoquinolinylene, 5,6,7,8-tetrahydroquinolinylene, 5,6,7,8-tetrahydroisoquinolinylene and phthalimidylene.

Other examples of heteroarylene groups are divalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroarylene groups are five- and six-membered divalent derivatives, such as the divalent derivatives of furan, imidazole, isothiazole, isoxazole, pyran, pyrazine, pyrazole, pyridazine, pyridine, pyrimidine, pyrrole, pyrrolizine and thiophene. The five-membered divalent derivatives are particularly preferred, i.e. the divalent derivatives of furan, imidazole, isothiazole, isoxazole, pyrazole, pyrrole and thiophene.

The term “heteroalkyl” includes alkyl groups in which up to three carbon atoms, preferably up to two carbon atoms, more preferably one carbon atom, are each replaced independently by O, S or N.

The term “heterocycloalkyl” includes cycloalkyl groups in which up to three carbon atoms, preferably up to two carbon atoms, more preferably one carbon atom, are each replaced independently by O, S or N. A preferred heterocycloalkyl group is morpholino.

The term “heteroalkenyl” includes alkenyl groups in which up to three carbon atoms, preferably up to two carbon atoms, more preferably one carbon atom, are each replaced independently by O, S or N.

The term “heterocycloalkenyl” includes cycloalkenyl groups in which up to three carbon atoms, preferably up to two carbon atoms, more preferably one carbon atom, are each replaced independently by O, S or N.

The term “heteroalkynyl” includes alkynyl groups in which up to three carbon atoms, preferably up to two carbon atoms, more preferably one carbon atom, are each replaced independently by O, S or N.

The term “heteroalkylene” includes alkylene groups in which up to three carbon atoms, preferably up to two carbon atoms, more preferably one carbon atom, are each replaced independently by O, S or N.

The term “heterocycloalkylene” includes cycloalkylene groups in which up to three carbon atoms, preferably up to two carbon atoms, more preferably one carbon atom, are each replaced independently by O, S or N.

The term “heteroalkenylene” includes alkenylene groups in which up to three carbon atoms, preferably up to two carbon atoms, more preferably one carbon atom, are each replaced independently by O, S or N.

The term “heterocycloalkenylene” includes alkenylene groups in which up to three carbon atoms, preferably up to two carbon atoms, more preferably one carbon atom, are each replaced independently by O, S or N.

The term “heteroalkynylene” includes alkynylene groups in which up to three carbon atoms, preferably up to two carbon atoms, more preferably one carbon atom, are each replaced independently by O, S or N.

Where reference is made to a carbon atom of an alkyl group or other group being replaced by an O, S, or N atom, what is intended is that:

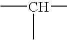

is replaced by

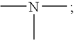

—CH═ is replaced by —N═; or
—CH₂— is replaced by —O—, —S— or —NR⁶—, where R⁶ is H, alkyl, aryl, —C(O)-alkyl, —C(O)-aryl, —S(O)₂-alkyl or —S(O)₂-aryl. R⁶ is preferably H or alkyl (e.g. C₁₋₆alkyl).

### Substitution

The alkyl, cycloalkyl, alkoxy, alkylthio, alkenyl, cycloalkenyl, alkynyl, alkylene, cycloalkylene, alkenylene, cycloalkenylene, alkynylene, aryl, arylalkyl, arylene, heteroaryl, heteroarylalkyl, heteroarylene, heteroalkyl, heterocycloalkyl, heteroalkenyl, heterocycloalkenyl, heteroalkynyl, heteroalkylene, heterocycloalkylene, heteroalkenylene, heterocycloalkenylene, and heteroalkynylene groups of the compounds of the invention may be substituted or unsubstituted, preferably unsubstituted.

Where substituted, there will generally be 1 to 3 substituents, preferably 1 or 2 substituents, more preferably 1 substituent. Preferred substituents are Sub¹, where Sub¹ is independently halogen, trihalomethyl, —NO₂, —CN, —N⁺(R^{s})₂O⁻, —CO₂H, —CO₂R^{s}, —SO₃H, —SOR^{s}, —SO₂R^{s}, —SO₃R^{s}, —OC(═O)OR^{s}, —C(═O)H, —C(═O)R^{s}, —OC(═O)R^{s}, —NR^{s}₂, —C(═O)NH₂, —C(═O)NR^{s}₂, —N(R^{s})C(═O)OR^{s}, —N(R^{s})C(═O)NR^{s}₂, —OC(═O)NR^{s}₂, —N(R^{s})C(═O)R^{s}, —C(═S)NR^{s}₂, —NR^{s}C(═S)R^{s}, —SO₂NR^{s}₂, —NR^{s}SO₂R^{s}, —N(R^{s})C(═S)NR^{s}₂, —N(R^{s})SO₂NR^{s}₂, —R^{s} or -Z^{s}R^{s}. Z^{s} is independently O, S or NR^{s}; R^{s} is independently H or C₁₋₆alkyl, C₃₋₆cycloalkyl, C₂₋₆alkenyl, C₃₋₆cycloalkenyl, C₃₋₆alkynyl, C₆₋₁₄aryl, heteroaryl having 5-13 members, C₆₋₁₄arylC₁₋₆alkyl, or heteroarylC₁₋₆alkyl where the heteroaryl has 5-13 members, where R^{s} is optionally substituted itself (preferably unsubstituted) by 1 to 3 substituents Sub², where Sub² is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z^{t}C₁₋₆alkyl, where Z^{t} is O, S or N(C₁₋₆alkyl).

Preferably, R^{s} is H or C₁₋₆alkyl, optionally substituted by 1 to 3 substituents Sub².

In addition, where a group has at least 2 positions which may be substituted, the group may be substituted by both ends of an alkylene or heteroalkylene chain (e.g. on the same carbon atom of the group) to form a cyclic moiety.

Where a phenyl group or a six-membered ring heteroaryl group (e.g. pyridyl) is substituted, substitution at the meta and/or para positions is preferred, with para substitution being especially preferred.

### General

The term “comprising” means “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### MODES FOR CARRYING OUT THE INVENTION

### Materials and Methods

400M Hz ¹H nuclear magnetic resonance (NMR) spectra were recorded on a Bruker Avance spectrometer. In the nuclear magnetic resonance (NMR) spectra the chemical shifts (δ) are expressed in ppm relative to the residual solvent peak. Abbreviations have the following significances: b=broad signal, s=singlet; d=doublet; t=triplet; m=multiplet; q=quartet; dd=doublet of doublets; ddd=doublet of double doublets. Abbreviations may be compounded and other patterns are unabbreviated.

The thin layer chromatography (TLC) R_{F} values were determined using Merck silica plates.

High Pressure Liquid Chromatography-Mass Spectrometry (LC-MS) conditions for determination of retention times (R_{T}) and associated mass ions were as follows. Mass Spectrometer (MS): Waters ZQ (Waters Ltd) Serial No. LAA623 Ionisation Mode: Electrospray (Positive Ion); Full Scan m/z 100-900, scanning for 0.6 sec with an interscan delay of 0.4 sec in centroid Mode. Electrospray (Negative Ion); Full Scan m/z 100-900, scanning for 0.6 sec with an interscan delay of 0.4 sec in centroid mode. Liquid Chromatograph (LC): Agilent 1100 series binary pump (Serial #DE33214258), degasser (Serial #JP13211877) & well plate auto sampler (Serial #DE33402913). Phenomenex Luna C18(2), 3μ (4.6 mm×150 mm) reverse phase column operated under gradient elution conditions using the methods and solvents described below.

### Method A

(A) Water containing 0.1% formic acid and (B) acetonitrile containing 0.1% formic acid as the mobile phase (gradient: 0.00 minutes, 95% A; linear gradient to 100% B at 12 minutes; then hold until 13.15 minutes). Flow rate 1 mil/minute to column & to UV detector, flow split after UV detector such that 0.25 ml/minute to MS detector and 0.75 ml/minute to waste; injection volume 5 μl; Auxiliary Detectors: —Agilent 1100 Series variable wavelength UV detector (serial #JP33322024) wavelength=220 nm.

### Method B

(A) Water containing 0.1% formic acid and (B) acetonitrile containing 0.1% formic acid as the mobile phase (gradient: 0.00 minutes, 80% A; linear gradient to 100% B at 12 minutes; then hold until 13.15 minutes). Flow rate 1 mil/minute to column & to UV detector, flow split after UV detector such that 0.25 ml/minute to MS detector and 0.75 ml/minute to waste; injection volume 5 μl; Auxiliary Detectors: —Agilent 1100 Series variable wavelength UV detector (serial #JP33322024) wavelength=220 nm.

### Method C

(A) Water containing 0.1% formic acid and (B) acetonitrile containing 0.1% formic acid as the mobile phase (gradient: 0.00 minutes, 60% A; linear gradient to 100% B at 12 minutes; then hold until 13.15 minutes). Flow rate 1 ml/minute to column & to UV detector, flow split after UV detector such that 0.25 ml/minute to MS detector and 0.75 ml/minute to waste; injection volume 5 μl; Auxiliary Detectors: —Agilent 1100 Series variable wavelength UV detector (serial #JP33322024) wavelength=220 nm.

### Method D

(A) Water containing 0.1% ammonium formate and (B) acetonitrile containing 0.1% ammonium formate as the mobile phase (gradient: 0.00 minutes, 80% A; linear gradient to 100% B at 12 minutes; then hold until 13.15 minutes). Flow rate 1 ml/minute to column & to UV detector, flow split after UV detector such that 0.25 ml/minute to MS detector and 0.75 ml/minute to waste; injection volume 5 μl; Auxiliary Detectors: —Agilent 1100 Series variable wavelength UV detector (serial #JP33322024) wavelength=220 nm. The abbreviations as used in the examples have the following meaning:
DMF: N,N-dimethylformamide
min.: minutes
EtOAc: ethyl acetate
R_{T}: retention time
eq.: equivalent
h: hour
CDCl3: deutorated chloroform
DMSO: dimethyl sulfoxide

### Preparation

Compounds of the invention may be conveniently prepared as described below.

Benzoylacetonitrile starting materials were purchased from commercial sources, or prepared from either the corresponding benzoyl chloride or alkyl benzoate.

From the benzoyl chloride:

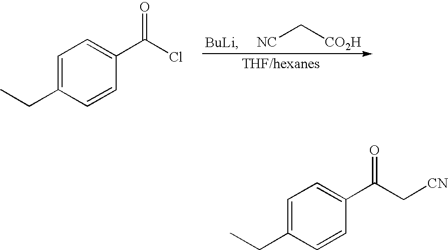

Cyanoacetic acid (21.27 g, 0.25 moles) is dissolved in anhydrous tetrahydrofuran (300 mL) and cooled to −78° C. under nitrogen. n-Butyllithium (177 mL of a 2.82 M solution in hexanes, 0.5 moles) is added slowly before the reaction is warmed to 0° C. and stirred for 30 minutes. The reaction is then recooled to −78° C. and a solution of 4-ethylbenzoyl chloride (21.1 g, 125 mmol) in anhydrous tetrahydrofuran (100 mL) added dropwise. The reaction is stirred for 1 hour and allowed to warm to room temperature then stirred for a further 1 hour. 1M hydrochloric acid (250 mL) is added slowly and the mixture extracted with DCM (3×200 mL). The combined organic phases are washed with brine (200 mL), dried over sodium sulphate, filtered and concentrated in vacuo. The residue is purified by flash column chromatography eluting with petroleum ether/diethyl ether (30/70), followed by recrystallisation from cyclohexane providing 5.198 g (24% yield) of the cyanoketone.

From the alkyl benzoate:

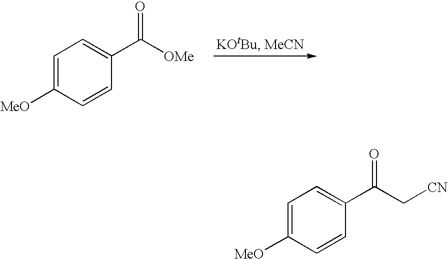

A solution of methyl p-anisate (33.2 g, 0.2 moles) in acetonitrile (140 mL) is treated with potassium tert-butoxide (24.4 g, 0.2 moles) and the slurry heated at 70° C. for 3.5 h. After cooling, most of the solvent is removed in vacuo. The residue is dissolved in water (250 mL) and washed with dichloromethane (2×100 mL). The aqueous solution is acidified to pH 8 with concentrated hydrochloric acid (20 mL) providing a precipitate which is filtered washed with water and dried. The crude solid is slurried in hot diethyl ether, filtered and dried providing a light beige solid (19.9 g, 57% yield).

Cyanoketones which do not precipitate from the aqueous phase on acidification can be isolated by extraction of the aqueous phase with ethyl acetate, followed by concentration of the organic extract.

### Example 1

### {[5-Ethyl-3-(4-trifluoromethoxy-benzoyl)-thiophen-2-ylcarbamoyl]-methylsulfanyl}-acetic acid

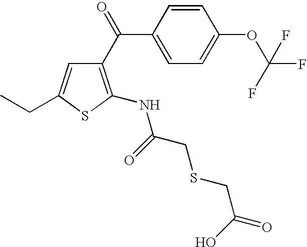

### Step 2: (2-Amino-5-ethyl-thiophen-3-yl)-(4-trifluoromethoxy-phenyl)-methanone

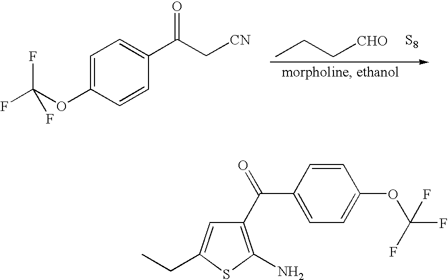

The following can be regarded as a general procedure for the synthesis of the aminothiophene intermediates from the required cyanoketone and aldehyde.

A suspension of (4-trifluoromethoxybenzoyl)acetonitrile (6.0 g, 26.2 mmol, 1 eq.) and sulphur (1.26 g, 39.3 mmol, 1.5 eq.) in ethanol (15 mL) and morpholine (7.5 mL) is treated with butyraldehyde (2.36 mL, 26.2 mmol, 1 eq.) and the suspension heated at 75° C. for 1.5 h. After the solution is allowed to cool, the solvent is removed in vacuo and the residue purified by column chromatography (1:4 ethyl acetate/petroleum ether) providing 5.71 g of a waxy yellow solid. This solid was purified further by trituration in petroleum ether, filtration and drying. A pale yellow powder was obtained (4.41 g, 53% yield).

### Step 3: {[5-Ethyl-3-(4-trifluoromethoxy-benzoyl)-thiophen-2-ylcarbamoyl]-methylsulfanyl}-acetic acid

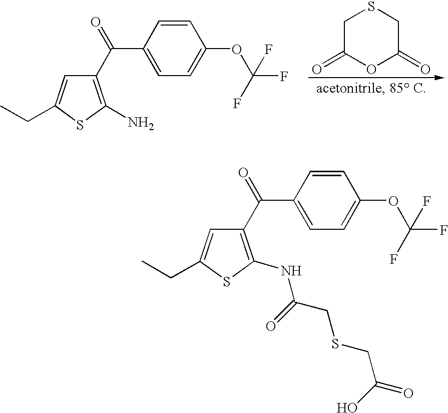

The following can be regarded as a general procedure for the acylation of an aminothiophene with a cyclic anhydride.

A solution of (2-amino-5-ethyl-thiophen-3-yl)-(4-trifluoromethoxy-phenyl)-methanone (1.10 g, 3.5 mmol) and thiodiglycolic anhydride (615 mg, 4.7 mmol) in acetonitrile (5 mL) is heated at reflux for 18 h. After cooling, the solution is diluted with diethyl ether and washed three times with water and once with brine. The ethereal solution is dried over sodium sulphate, filtered and concentrated to dryness. The crude yellow gum is obtained as a solid by trituration in methanol/diethyl ether/petroleum ether and concentration in vacuo. The solid is purified by trituration in diethyl ether/petroleum ether (1:5), filtration and drying, providing a yellow powder (1.32 g, 84% yield).

¹H NMR (400 MHz, DMSO-d6) δ=12.23 (1H, bs), 7.86 (2H, d, J=9 Hz), 7.55 (2H, d, J=9 Hz), 6.84 (1H, s), 3.73 (2H, s), 3.40 (2H, s), 2.74 (2H, q, J=6 Hz), 1.21 (3H, t, J=6 Hz).

LCMS (Method A): R_{T}=11.78 min. m/z=448 (ES+, M+H), 446 (ES−, M−H)

Analogues of this compound can also be purified by column chromatography in ethyl acetate, containing methanol or acetic acid as polar additives.

Reaction of the aminothiophene with a cyclic anhydride can also be performed in toluene.

### Example 2

### 2-{[5-Ethyl-3-(4-trifluoromethoxy-benzoyl)-thiophen-2-ylcarbamoyl]-methylsulfanyl}-2-methyl-propionic acid

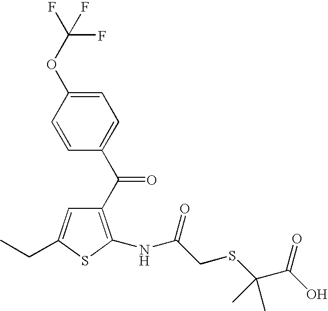

The title compound was made by an analogous procedure to Example 1, using 3,3-dimethyl-[1,4]oxathiane-2,6-dione in the final step.

¹H NMR (400 MHz, CDCl₃) δ=12.60 (1H, bs), 7.78 (2H, d, J=8.8 Hz), 7.31 (2H, d, J=8.8 Hz), 6.73 (1H, s), 3.68 (2H, s), 2.74 (2H, q, J=7.6 Hz), 1.56 (6H, s), 1.27 (3H, t, J=7.6 Hz)

LCMS (Method A) R_{T}=9.58 min. m/z=476 (ES+, M+H), 474 (ES−, M−H)

### Example 3

### 4-[5-Ethyl-3-(4-trifluoromethoxy-benzoyl)-thiophen-2-ylcarbamoyl]-3,3-dimethyl-butylic acid

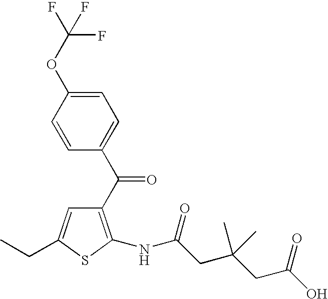

The title compound was made by an analogous procedure to Example 1, using 3,3-dimethylglutaric anhydride in the final step.

¹H NMR (400 MHz, CDCl₃) δ=12.01 (1H, bs), 7.75 (2H, d, J=8.8 Hz), 7.31 (2H, d, J=8.8 Hz), 6.73 (1H, s), 2.74 (2H, q, J=7.6 Hz), 2.66 (2H, s), 2.50 (2H, s), 1.28 (3H, t, J=7.8 Hz), 1.20 (6H, s)

LCMS (Method A) R_{T}=12.27 min. m/z=458 (ES+, M+H), 456 (ES−, M−H)

### Example 4

### (1-{[5-Ethyl-3-(4-trifluoromethoxy-benzoyl)-thiophen-2-ylcarbamoyl]-methyl}-cyclopentyl)-acetic acid

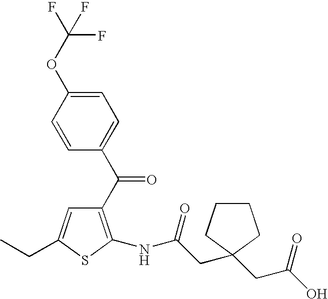

The title compound was made by an analogous procedure to Example 1, using 8-oxa-spiro[4.5]decane-7,9-dione in the final step.

¹H NMR (400 MHz, CDCl₃) δ=12.05 (1H, bs), 7.75 (2H, d, J=8.7 Hz), 7.31 (2H, d, J=8.7 Hz), 6.73 (1H, s), 2.78-2.71 (4H, m), 2.56 (2H, s), 1.74-1.65 (8H, m), 1.28 (3H, t, J=7.6 Hz)

LCMS (Method A) R_{T}=13.00 min. m/z=484 (ES+, M+H), 482 (ES−, M−H)

### Example 5

### 2-{[5-Ethyl-3-(4-methoxy-benzoyl)-thiophen-2-ylcarbamoyl]-methylsulfanyl}-2-methyl-propionic acid

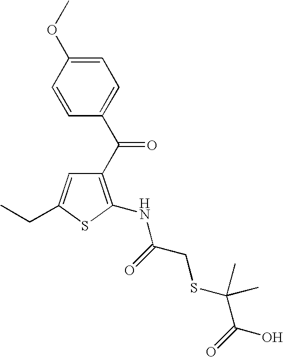

The title compound was prepared from (4-methoxybenzoyl)acetonitrile by an analogous procedure to Example 1, but using 3,3-dimethyl-[1,4]oxathiane-2,6-dione in the final step.

¹H NMR (400 MHz, CDCl₃) δ=12.58 (1H, bs), 7.75 (2H, d, J=8.7 Hz), 6.97 (2H, d, J=8.7 Hz), 6.81 (1H, s), 3.89 (3H, s), 3.68 (2H, s), 2.74 (2H, q, J=7.5 Hz), 1.57 (3H, s), 1.57 (3H, s), 1.28 (3H, t, J=7.5 Hz)

LCMS (Method A): R_{T}=11.41 min. m/z=422 (ES+, M+H), 420 (ES−, M−H)

### Example 6

### (1-{[5-Ethyl-3-(4-methoxy-benzoyl)-thiophen-2-ylcarbamoyl]-methyl}-cyclopentyl)-acetic acid

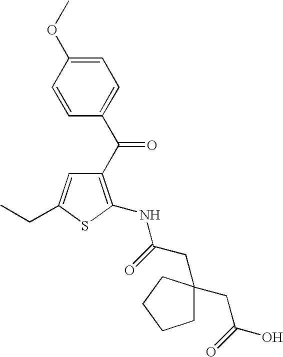

The title compound was made by an analogous procedure to Example 5, using 8-oxa-spiro[4.5]decane-7,9-dione in the final step.

¹H NMR (400 MHz, CDCl₃) δ=12.11 (1H, bs), 7.72 (2H, d, J=8.8 Hz), 6.96 (2H, d, J=8.8 Hz), 6.81 (1H, s), 3.88 (3H, s), 2.78-2.71 (4H, m), 2.55 (2H, s), 1.74-1.63 (8H, m), 1.28 (3H, t, J=7.7 Hz)

LCMS (Method A): R_{T}=12.47 min. m/z=430 (ES+, M+H), 429 (ES−, M−H)

### Example 7

### 4-[5-Ethyl-3-(4-methoxy-benzoyl)-thiophen-2-ylcarbamoyl]-butyric acid

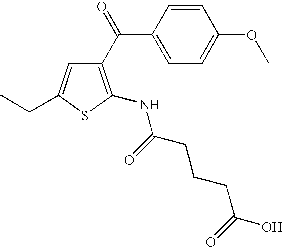

The title compound was made by an analogous procedure to Example 5, using glutaric anhydride in the final step.

¹H NMR (400 MHz, CDCl₃) δ=11.98 (1H, s), 7.73 (2H, d, J=8.8 Hz), 6.97 (2H, d, J=8.8 Hz), 6.79 (1H, s), 3.88 (3H, s), 2.74 (2H, q, J=7.5 Hz), 2.62 (2H, t, J=7.3 Hz), 2.50 (2H, t, J=7.3 Hz), 2.10 (2H, q, J=7.3 Hz), 1.27 (3H, t, J=7.5 Hz).

LCMS (Method A): R_{T}=10.57 min. m/z=376 (ES+, M+H), 374 (ES−, M−H)

### Example 8

### {[5-Ethyl-3-(4-methyl-benzoyl)-thiophen-2-ylcarbamoyl]-methylsulfanyl}-acetic acid

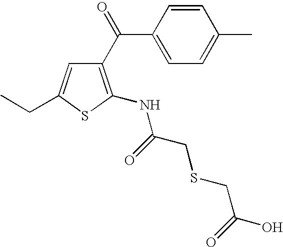

The title compound was prepared from (4-methylbenzoyl)acetonitrile by an analogous procedure to Example 1.

¹H NMR (400 MHz, DMSO-d6) δ=12.23 (1H, bs), 7.63 (2H, d, J=8 Hz), 7.37 (2H, d, J=8 Hz), 6.83 (1H, s), 3.72 (2H, s), 3.41 (2H, s), 2.74 (2H, q, J=7 Hz), 2.41 (3H, s), 1.21 (3H, t, J=7 Hz).

LCMS (Method A): R_{T}=11.23 min. m/z=378 (ES+, M+H), 376 (ES−, M−H)

### Example 9

### {[5-Ethyl-3-(4-ethyl-benzoyl)-thiophen-2-ylcarbamoyl]-methylsulfanyl}-acetic acid

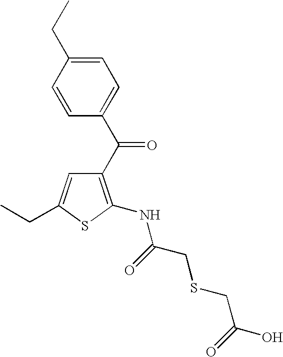

The title compound was prepared from (4-ethylbenzoyl)acetonitrile by an analogous procedure to Example 1.

¹H NMR (400 MHz, CDCl₃) δ=12.53 (1H, bs), 7.60 (2H, d, J=8 Hz), 7.25 (2H, d, J=8 Hz), 6.76 (1H, s), 3.60 (2H, s), 3.36 (2H, s), 2.69 (2H, q, J=8 Hz), 2.69 (2H, q, J=8 Hz), 1.22 (3H, t, J=8 Hz), 1.22 (3H, t, J=8 Hz).

LCMS (Method A): R_{T}=11.85 min. m/z=392 (ES+, M+H), 390 (ES−, M−H)

### Example 10

### 2-{[5-Ethyl-3-(4-ethyl-benzoyl)-thiophen-2-ylcarbamoyl]-methylsulfanyl}-2-methyl-propionic acid

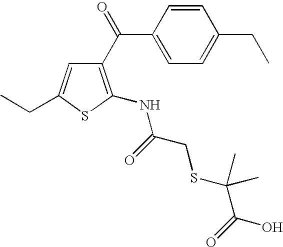

The title compound was prepared by an analogous procedure to Example 9, using 3,3-dimethyl-[1,4]oxathiane-2,6-dione in the final step.

¹H NMR (400 MHz, DMSO-d6) δ=7.66 (2H, d, J=8 Hz), 7.40 (2H, d, J=8 Hz), 6.85 (1H, s), 3.75 (2H, s), 2.77-2.67 (4H, m), 1.43 (6H, s), 1.24 (3H, t, J=8 Hz), 1.21 (3H, t, J=8 Hz).

LCMS (Method B): R_{T}=12.14 min. m/z=420 (ES+, M+H), 418 (ES−, M−H)

### Example 11

### (1-{[5-Ethyl-3-(4-ethyl-benzoyl)-thiophen-2-ylcarbamoyl]-methyl}-cyclopentyl)-acetic acid

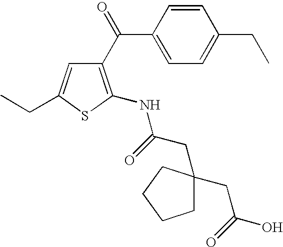

The title compound was prepared by an analogous procedure to Example 9, using 8-oxa-spiro[4.5]decane-7,9-dione in the final step.

¹H NMR (400 MHz, DMSO-d6) δ=7.64 (2H, d, J=8 Hz), 7.39 (2H, d, J=8 Hz), 6.81 (1H, s), 2.75-2.67 (4H, m) 2.74 (2H, s), 2.40 (2H, s), 1.66-1.55 (8H, m), 1.23 (2H, t, J=8 Hz), 1.21 (3H, t, J=7 Hz).

LCMS (Method B): R_{T}=13.30 min. m/z=428 (ES+, M+H), 426 (ES−, M−H)

### Example 12

### {[5-Ethyl-3-(4-phenoxy-benzoyl)-thiophen-2-ylcarbamoyl]-methylsulfanyl}-acetic acid

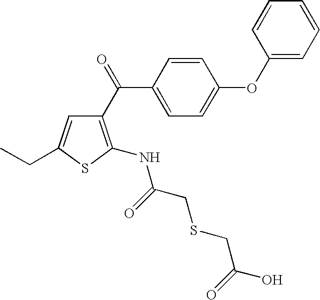

The title compound was prepared from (4-phenoxybenzoyl)acetonitrile by an analogous procedure to Example 1.

¹H NMR (400 MHz, DMSO-d6) δ=12.19 (1H, bs), 7.78 (2H, d, J=9 Hz), 7.49 (2H, t, J=7 Hz), 7.26 (1H, t, J=7 Hz), 7.17 (2H, d, J=7 Hz), 7.09 (2H, d, J=9 Hz), 6.88 (1H, s), 3.72 (2H, s), 3.41 (2H, s), 2.74 (2H, q, J=8 Hz), 1.22 (3H, t, J=8 Hz).

LCMS (Method B): R_{T}=11.61 min. m/z=456 (ES+, M+H), 454 (ES−, M−H)

### Example 13

### 4-[5-Ethyl-3-(4-phenoxy-benzoyl)-thiophen-2-ylcarbamoyl]-3,3-dimethyl-butyric acid

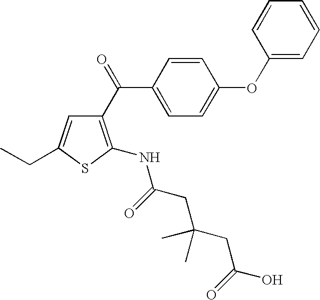

The title compound was made by an analogous procedure to Example 12, using 3,3-dimethylglutaric anhydride in the final step.

¹H NMR (400 MHz, CDCl₃) δ=12.06 (1H, bs), 7.66 (2H, d, J=9 Hz), 7.34 (2H, t, J=9 Hz), 7.14 (1H, t, J=8 Hz), 7.03 (2H, d, J=8 Hz), 6.97 (2H, d, J=8 Hz), 6.76 (1H, s), 2.69 (2H, q, J=7 Hz), 2.57 (2H, s), 2.44 (2H, s), 1.22 (3H, t, J=7 Hz), 1.14 (6H, s).

LCMS (Method B): R_{T}=12.74 min. m/z=466 (ES+, M+H), 464 (ES−, M−H)

### Example 14

### [(3-Benzoyl-5-ethyl-thiophen-2-ylcarbamoyl)-methylsulfanyl]-acetic acid

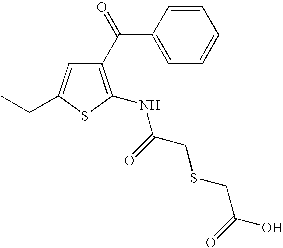

The title compound was prepared from benzoylacetonitrile by an analogous procedure to Example 1.

¹H NMR (400 MHz, DMSO-d6) δ=12.27 (1H, bs), 7.73-7.53 (5H, m), 6.81 (1H, s), 3.73 (2H, s), 3.40 (2H, s), 2.76 (2H, q, J=7 Hz), 1.21 (3H, t, J=7 Hz).

LCMS (Method A): R_{T}=10.57 min. m/z=364 (ES+, M+H), 362 (ES−, M−H)

### Example 15

### 2-[(3-Benzoyl-5-ethyl-thiophen-2-ylcarbamoyl)-methylsulfanyl]-2-methyl-propionic acid

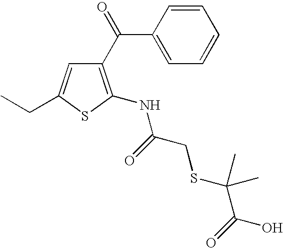

The title compound was made by an analogous procedure to Example 14, using 3,3-dimethyl-[1,4]oxathiane-2,6-dione in the final step.

¹H NMR (400 MHz, DMSO-d6) δ=7.74-7.69 (2H, m), 7.64 (1H, tt, J=8, 2 Hz), 7.59-7.53 (2H, m), 6.82 (1H, s), 3.76 (2H, s), 2.74 (2H, qd, J=8, 1 Hz), 1.44 (6H, s), 1.21 (3H, t, J=8 Hz).

LCMS (Method A): R_{T}=11.55 min. m/z=392 (ES+, M+H), 390 (ES−, M−H)

### Example 16

### 4-(3-Benzoyl-5-ethyl-thiophen-2-ylcarbamoyl)-3,3-dimethyl-butyric acid

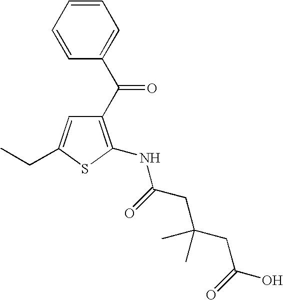

The title compound was made by an analogous procedure to Example 14, using 3,3-dimethylglutaric anhydride in the final step.

¹H NMR (400 MHz, DMSO-d6) δ=12.16 (1H, bs), 7.72-7.68 (2H, m), 7.64 (1H, tt, J=7, 2 Hz), 7.56 (2H, t, J=7 Hz), 6.79 (1H, s), 2.73 (2H, q, J=8 Hz), 2.65 (2H, s), 2.32 (2H, s), 1.20 (3H, t, J=8 Hz), 1.10 (6H, s).

LCMS (Method A): R_{T}=11.80 min. m/z=374 (ES+, M+H), 372 (ES−, M−H)

### Example 17

### {1-[(3-Benzoyl-5-ethyl-thiophen-2-ylcarbamoyl)-methyl]-cyclopentyl}-acetic acid

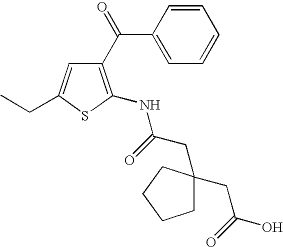

The title compound was made by an analogous procedure to Example 14, using 8-oxa-spiro[4.5]decane-7,9-dione in the final step.

¹H NMR (400 MHz, DMSO-d6) δ=12.18 (1H, bs), 7.72-7.66 (2H, m), 7.64 (1H, tt, J=7, 2 Hz), 7.60-7.52 (2H, m), 6.79 (1H, s), 2.76 (2H, s), 2.73 (2H, q, J=7 Hz), 2.40 (2H, s), 1.67-1.54 (8H, m), 1.20 (3H, t, J=7 Hz).

LCMS (Method A): R_{T}=12.60 min. m/z=400 (ES+, M+H), 398 (ES−, M−H)

### Example 18

### {[5-Ethyl-3-(4-fluoro-benzoyl)-thiophen-2-ylcarbamoyl]-methylsulfanyl}-acetic acid

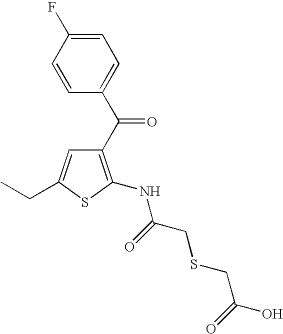

The title compound was prepared from (4-fluorobenzoyl)acetonitrile by an analogous procedure to Example 1.

¹H NMR (400 MHz, CDCl₃) δ=12.48 (1H, bs), 7.72-7.67 (2H, m), 7.11 (2H, t, J=4 Hz), 6.70 (1H, s), 3.60 (2H, s), 3.36 (2H, s), 2.69 (2H, q, J=8 Hz), 1.22 (3H, t, J=8 Hz).

LCMS (Method A): R_{T}=10.73 min. m/z=382 (ES+, M+H), 380 (ES−, M−H)

### Example 19

### 2-{[5-Ethyl-3-(4-fluoro-benzoyl)-thiophen-2-ylcarbamoyl]-methylsulfanyl}-2-methyl-propionic acid

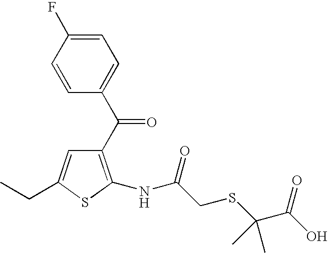

The title compound was made by an analogous procedure to Example 18, using 3,3-dimethyl-[1,4]oxathiane-2,6-dione in the final step.

¹H NMR (400 MHz, CDCl₃) δ=12.61 (1H, bs), 7.75 (2H, dd, J=8.9, 5.5 Hz), 7.16 (2H, t, J=8.9 Hz), 6.73 (1H, t, J=0.8 Hz), 3.70 (2H, s), 2.73 (2H, dq, J=7.2, 0.8 Hz), 1.56 (6H, s), 1.27 (3H, t, J=7.2 Hz)

LCMS (Method A): R_{T}=10.87 min. m/z=410 (ES+, M+H), 408 (ES−, M−H)

### Example 20

### 4-[5-Ethyl-3-(4-fluoro-benzoyl)-thiophen-2-ylcarbamoyl]-3,3-dimethyl-butyric acid

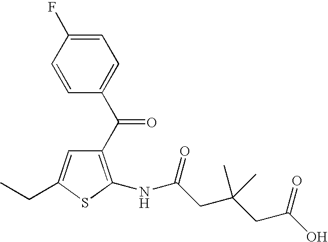

The title compound was made by an analogous procedure to Example 18, using 3,3-dimethylglutaric anhydride in the final step.

¹H NMR (400 MHz, CDCl₃) δ=12.04 (1H, bs), 7.73 (2H, dd, J=8.7, 5.5 Hz), 7.16 (2H, t, J=8.3 Hz), 6.74 (1H, s), 2.75 (2H, s), 2.65 (2H, s), 1.28 (3H, t, J=7.7 Hz), 1.20 (6H, s)

LCMS (Method A): R_{T}=11.17 min. m/z=392 (ES+, M+H), 390 (ES−, M−H)

### Example 21

### (1-{[5-Ethyl-3-(4-fluoro-benzoyl)-thiophen-2-ylcarbamoyl]-methyl}-cyclopentyl)-acetic acid

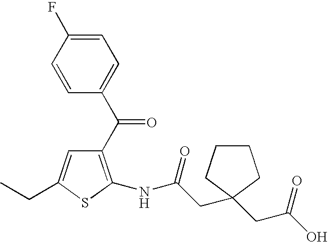

The title compound was made by an analogous procedure to Example 18, using 8-oxa-spiro[4.5]decane-7,9-dione in the final step.

¹H NMR (400 MHz, CDCl₃) δ=12.01 (1H, bs), 7.71 (2H, dd, J=8.8, 5.6 Hz), 7.15 (2H, t, J=8.8 Hz), 6.73 (1H, s), 2.75-2.70 (4H, m), 2.57 (2H, s), 1.80-1.60 (8H, m), 1.28 (3H, t, J=7.5 Hz)

LCMS (Method A): R_{T}=12.05 min. m/z=418 (ES+, M+H), 416 (ES−, M−H)

### Example 22

### {[3-(4-Bromo-benzoyl)-5-ethyl-thiophen-2-ylcarbamoyl]-methylsulfanyl}-acetic acid

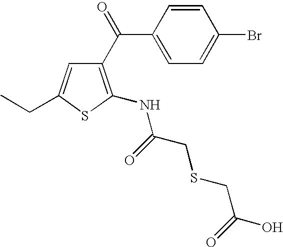

The title compound was prepared from (4-bromobenzoyl)acetonitrile by an analogous procedure to Example 1.

¹H NMR (400 MHz, CDCl₃) δ=12.47 (1H, bs), 7.57 (2H, d, J=9 Hz), 7.53 (2H, d, J=9 Hz), 6.67 (1H, s), 3.60 (2H, s), 3.36 (2H, s), 2.68 (2H, q, J=9 Hz), 1.22 (3H, t, J=9 Hz).

LCMS (Method A): R_{T}=11.74 min. m/z=442/444 (ES+, M+H), 440/442 (ES−, M−H)

### Example 23

### {[3-(4-Chloro-benzoyl)-5-ethyl-thiophen-2-ylcarbamoyl]-methylsulfanyl}-acetic acid

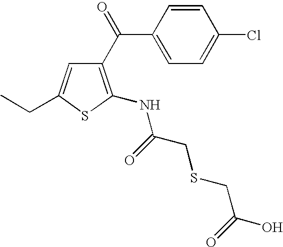

The title compound was prepared from (4-chlorobenzoyl)acetonitrile by an analogous procedure to Example 1.

¹H NMR (400 MHz, DMSO-d6) δ=12.24 (1H, bs), 7.74 (2H, d, J=9 Hz), 7.63 (2H, d, J=9 Hz), 6.82 (1H, s), 3.73 (2H, s), 3.41 (2H, s), 2.74 (2H, q, J=7 Hz), 1.21 (3H, t, J=7 Hz).

LCMS (Method A): R_{T}=11.50 min. m/z=398/400 (ES+, M+H), 396/398 (ES−, M−H)

### Example 24

### 2-{[3-(4-Chloro-benzoyl)-5-ethyl-thiophen-2-ylcarbamoyl]-methylsulfanyl}-2-methyl-propionic acid

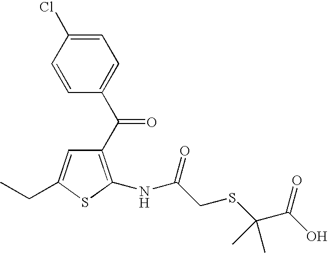

The title compound was made by an analogous procedure to Example 23, using 3,3-dimethyl-[1,4]oxathiane-2,6-dione in the final step.

¹H NMR (400 MHz, CDCl₃) δ=12.63 (1H, bs), 7.67 (2H, d, J=8.4 Hz), 7.45 (2H, d, J=8.4 Hz), 6.71 (1H, t, J=1.0 Hz), 3.69 (2H, s), 2.72 (2H, dq, J=7.5, 1.0 Hz), 1.57 (3H, s), 1.56 (3H, s), 1.26 (3H, t, J=7.5 Hz)

LCMS (Method A): R_{T}=11.76 min. m/z=428/426 (ES+, M+H₂0), 407/405 (ES−, M−H)

### Example 25

### 4-[3-(4-Chloro-benzoyl)-5-ethyl-thiophen-2-ylcarbamoyl]-3,3-dimethyl-butyric acid

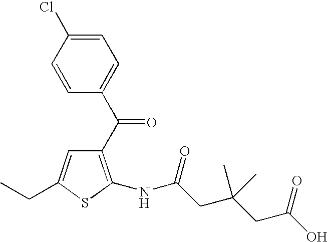

The title compound was made by an analogous procedure to Example 23, using 3,3-dimethylglutaric anhydride in the final step.

¹H NMR (400 MHz, CDCl₃) δ=12.04 (1H, bs), 7.65 (2H, d, J=8.3 Hz), 7.45 (2H, d, J=8.3 Hz), 6.77 (1H, t, J=0.8 Hz), 2.74 (2H, dq, J=7.4, 0.8 Hz), 2.65 (2H, s), 2.50 (2H, s), 1.28 (3H, t, J=7.4 Hz), 1.20 (6H, s)

LCMS (Method A): R_{T}=12.06 min. m/z=410/408 (ES+, M+H), 408/406 (ES−, M−H)

### Example 26

### (1-{[3-(4-Chloro-benzoyl)-5-ethyl-thiophen-2-ylcarbamoyl]-methyl}-cyclopentyl)-acetic acid

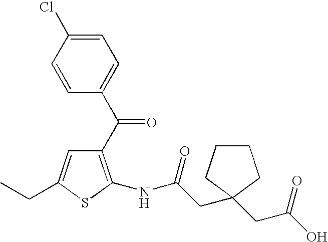

The title compound was made by an analogous procedure to Example 23, using 8-oxa-spiro[4.5]decane-7,9-dione in the final step.

¹H NMR (400 MHz, CDCl₃) δ=12.05 (1H, bs), 7.64 (2H, d, J=8.4 Hz), 7.45 (2H, d, J=8.4 Hz), 6.72 (1H, s), 2.77-2.71 (4H, m), 2.56 (2H, s), 1.77-1.64 (8H, m), 1.28 (3H, t, J=7.6 Hz)

LCMS (Method A): R_{T}=12.85 min. m/z=436/434 (ES+, M+H), 434/432 (ES−, M−H)

### Example 27

### {[3-(3-Chloro-benzoyl)-5-ethyl-thiophen-2-ylcarbamoyl]-methylsulfanyl}-acetic acid

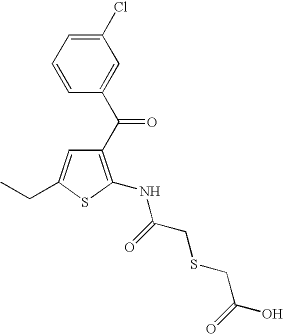

The title compound was prepared from (3-chlorobenzoyl)acetonitrile by an analogous procedure to Example 1.

¹H NMR (400 MHz, CDCl₃) δ=12.48 (1H, bs), 7.63 (1H, t, J=2 Hz), 7.52 (1H, d, J=8 Hz), 7.46 (1H, d, J=7 Hz), 7.36 (1H, t, J=8 Hz), 6.68 (1H, s), 3.60 (2H, s), 3.35 (2H, s), 2.69 (2H, q, J=8 Hz), 1.22 (3H, t, J=8 Hz).

LCMS (Method A): R_{T} 11.40 min. m/z=398/400 (ES+, M+H), 396/398 (ES−, M−H)

### Example 28

### {[3-(3,4-Dichloro-benzoyl)-5-ethyl-thiophen-2-ylcarbamoyl]-methylsulfanyl}-acetic acid

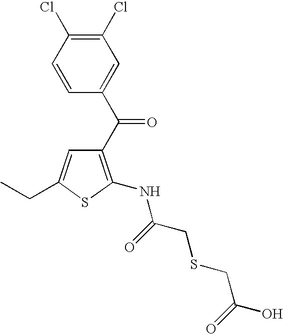

The title compound was prepared from (3,4-dichlorobenzoyl)acetonitrile by an analogous procedure to Example 1.

¹H NMR (400 MHz, CDCl₃) δ=12.44 (1H, bs), 7.75 (1H, d, J=2 Hz), 7.50 (2H, bs) 6.66 (1H, s), 3.60 (2H, s), 3.35 (2H, s), 2.69 (2H, qd, J=8, 1 Hz), 1.22 (3H, t, J=8 Hz).

LCMS (Method A): R_{T}=12.20 min. m/z=432/434/436 (ES+, M+H)

### Example 29

### 2-{[3-(3,4-Dichloro-benzoyl)-5-ethyl-thiophen-2-ylcarbamoyl]-methylsulfanyl}-2-methyl-propionic acid

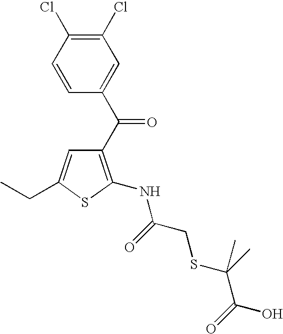

The title compound was made by an analogous procedure to Example 28, using 3,3-dimethyl-[1,4]oxathiane-2,6-dione in the final step.

¹H NMR (400 MHz, CDCl₃) δ=7.76 (1H, s), 7.50 (2H, s), 6.64 (1H, s), 3.62 (2H, s), 2.68 (2H, q, J=8 Hz), 1.52 (6H, s), 1.21 (3H, t, J=8 Hz).

LCMS (Method B): R_{T}=12.59 min. m/z=460/462/464 (ES+, M+H), 458/460/462 (ES−, M−H)

### Example 30

### 4-[3-(3,4-Dichloro-benzoyl)-5-ethyl-thiophen-2-ylcarbamoyl]-3,3-dimethyl-butyric acid

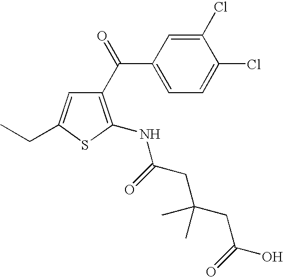

The title compound was made by an analogous procedure to Example 28, using 3,3-dimethylglutaric anhydride in the final step.

¹H NMR (400 MHz, DMSO-d6) δ=7.88 (1H, d, J=2 Hz), 7.83 (1H, d, J=8 Hz), 7.66 (1H, dd, J=8, 2 Hz), 6.79 (1H, s), 2.72 (2H, qd, J=8, 1 Hz), 2.65 (2H, s), 2.30 (2H, s), 1.20 (3H, t, J=8 Hz), 1.09 (6H, s).

LCMS (Method B): R_{T}=12.59 min. m/z=460/462/464 (ES+, M+H), 458/460/462 (ES−, M−H)

### Example 31

### {[3-(3-Chloro-4-fluoro-benzoyl)-5-ethyl-thiophen-2-ylcarbamoyl]-methylsulfanyl}-acetic acid

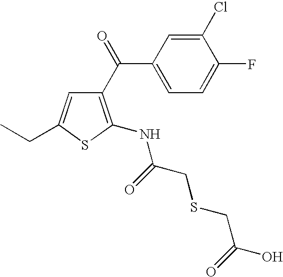

The title compound was prepared from (3-chloro-4-fluorobenzoyl)acetonitrile by an analogous procedure to Example 1.

¹H NMR (400 MHz, CDCl₃) δ=12.43 (1H, bs), 7.75 (1H, dd, J=7, 2 Hz), 7.57 (1H, ddd, J=8, 5, 2 Hz), 7.19 (1H, t, J=8 Hz), 6.67 (1H, s), 3.60 (2H, s), 3.36 (2H, s), 2.69 (2H, q, J=8 Hz), 1.23 (3H, t, J=8 Hz).

LCMS (Method B): R_{T}=10.77 min. m/z=416/418 (ES+, M+H), 414/416 (ES−, M−H)

### Example 32

### 4-[3-(3-Chloro-4-fluoro-benzoyl)-5-ethyl-thiophen-2-ylcarbamoyl]-3,3-dimethyl-butyric acid

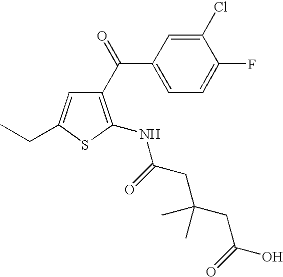

The title compound was made by an analogous procedure to Example 31, using 3,3-dimethylglutaric anhydride in the final step.

¹H NMR (400 MHz, CDCl₃) δ=11.97 (1H, bs), 7.74 (1H, dd, J=7, 2 Hz), 7.56 (1H, ddd, J=8, 5, 2 Hz), 7.19 (1H, s), 6.68 (1H, s), 2.70 (2H, q, J=6 Hz), 2.58 (2H, s), 2.44 (2H, s), 1.23 (3H, t, J=6 Hz), 1.14 (6H, s).

LCMS (Method B): R_{T}=12.09 min. m/z=424/426 (ES+, M+H), 422/424 (ES−, M−H)

### Example 33

### {[5-Ethyl-3-(4-isopropoxy-benzoyl)-thiophen-2-ylcarbamoyl]-methylsulfanyl}-acetic acid

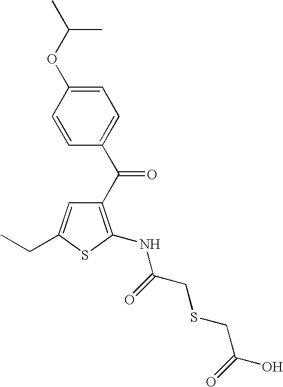

The title compound was prepared from (4-isopropoxybenzoyl)acetonitrile by an analogous procedure to Example 1.

¹H NMR (400 MHz, CDCl₃) δ=12.48 (1H, bs), 7.67 (2H, d, J=9 Hz), 6.88 (2H, d, J=9 Hz), 6.78 (1H, s), 4.59 (1H, septet, J=5 Hz), 3.59 (2H, s), 3.36 (2H, s), 2.69 (2H, q, J=7 Hz), 1.32 (6H, d, J 15=6 Hz), 1.22 (3H, t, J=8 Hz).

LCMS (Method A): R_{T}=11.71 min. m/z=422 (ES+, M+H), 420 (ES−, M−H)

### Example 34

### {[3-(3-Bromo-benzoyl)-5-ethyl-thiophen-2-ylcarbamoyl]-methylsulfanyl}-acetic acid

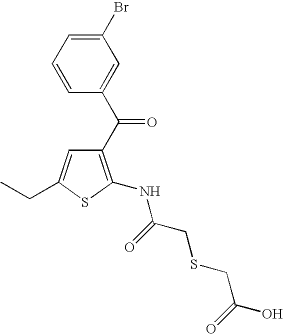

The title compound was prepared from (3-bromobenzoyl)acetonitrile by an analogous procedure to Example 1.

¹H NMR (400 MHz, CDCl₃) δ=12.48 (1H, bs), 7.67 (2H, d, J=9 Hz), 6.88 (2H, d, J=9 Hz), 6.78 (1H, s), 3.59 (2H, s), 3.36 (2H, s), 2.69 (2H, q, J=7 Hz), 1.22 (3H, t, J=8 Hz).

LCMS (Method A): R_{T}=10.88 min. m/z=442/444 (ES+, M+H), 440/442 (ES−, M−H)

### Example 35

### {[3-(4-Cyano-benzoyl)-5-ethyl-thiophen-2-ylcarbamoyl]-methylsulfanyl}-acetic acid

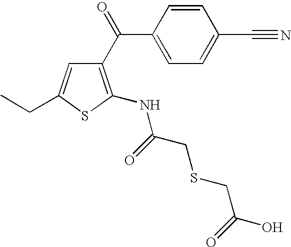

The title compound was prepared from (4-cyanobenzoyl)acetonitrile by an analogous procedure to Example 1.

¹H NMR (400 MHz, DMSO-d6) δ=12.71 (bs, H), 7.86-7.81 (m, 2H), 7.69 (ddd, 1H, J=8, 1, 1 Hz), 7.53 (dd, 1H, J=8, 8 Hz), 6.79 (s, 1H), 3.74 (s, 2H), 3.42 (s, 2H), 2.74 (q, 2H, J=7 Hz), 1.21 t, 3H, J=7 Hz).

LCMS (Method A): R_{T}=10.88 min. m/z=339 (ES+, M+H), 337 (ES−, M−H)

### Example 36

### {[3-(Biphenyl-4-carbonyl)-5-ethyl-thiophen-2-ylcarbamoyl]-methylsulfanyl}-acetic acid

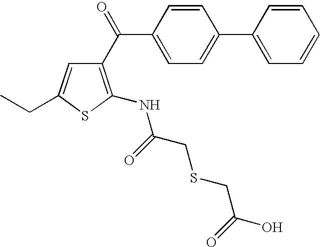

The title compound was prepared from (4-phenylbenzoyl)acetonitrile by an analogous procedure to Example 1.

¹H NMR (400 MHz, DMSO-d6) δ=12.26 (1H, bs), 7.88-7.77 (6H, m), 7.53 (2H, t, J=7 Hz), 7.45 (1H, t, J=7 Hz), 6.91 (1H, s), 3.74 (2H, s), 3.42 (2H, s), 2.76 (2H, q, J=6 Hz), 1.23 (3H, t, J=6 Hz).

LCMS (Method B): R_{T}=11.68 min. m/z=440 (ES+, M+H), 438 (ES−, M−H)

### Example 37

### 4-[3-(Biphenyl-4-carbonyl)-5-ethyl-thiophen-2-ylcarbamoyl]-3,3-dimethyl-butyric acid

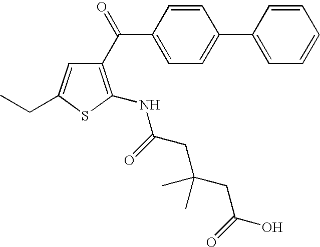

The title compound was made by an analogous procedure to Example 36, using 3,3-dimethylglutaric anhydride in the final step.

¹H NMR (400 MHz, CDCl₃) δ=12.11 (1H, bs), 7.23 (2H, d, J=9 Hz), 7.64 (2H, d, J=9 Hz), 7.58 (2H, d, J=7 Hz), 7.42 (2H, t, J=7 Hz), 7.34 (1H, t, J=7 Hz), 6.79 (1H, s), 2.70 (2H, q, J=8 Hz), 2.59 (2H, s), 2.45 (2H, s), 1.23 (3H, t, J=8 Hz), 1.45 (6H, s).

LCMS (Method B): R_{T}=12.87 min. m/z=450 (ES+, M+H), 448 (ES−, M−H)

### Example 38

### {[5-Ethyl-3-(4′-trifluoromethyl-biphenyl-4-carbonyl)-thiophen-2-ylcarbamoyl]-methylsulfanyl}-acetic acid

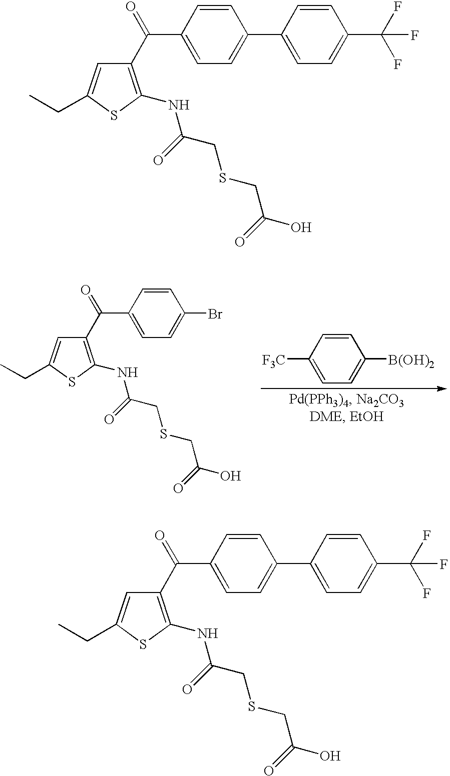

The aryl bromide (452 μmol) was dissolved in dimethoxyethane (2.88 ml) and ethanol (0.72 ml). The boronic acid (678 μmol) was added followed by 2M Na₂CO₃ (452 μl) and the suspension was degassed by gently bubbling N₂ through the mixture for 2 minutes. Pd(PPh₃)₄ (24 mg, 22 μmol) was added and the reaction was heated in a microwave reactor at 140° C. for four minutes. The reaction was then diluted with EtOAc (50 ml) and 0.5M HCl (25 ml). The organic phase was washed with saturated brine solution (20 ml), dried over sodium sulphate, filtered and concentrated in vacuo. The residue was purified by flash column chromatography on silica gel eluting with diethyl ether moving to diethyl ether plus one percent acetic acid.

¹H NMR (400 MHz, CDCl₃) δ=12.54 (1H, bs), 7.77 (2H, d, J=9 Hz), 7.68 (4H, bs), 7.65 (2H, d, J=9 Hz), 6.78 (1H, s,), 3.62 (2H, s), 3.38 (2H, s), 2.70 (2H, q, J=7 Hz), 1.23 (3H, t, J=7 Hz).

LCMS (Method A): R_{T}=12.42 min. m/z=508 (ES+, M+H), 506 (ES−, M−H)

### Example 39

### {[5-Ethyl-3-(4′-trifluoromethoxy-biphenyl-4-carbonyl)-thiophen-2-ylcarbamoyl]-methylsulfanyl}-acetic acid

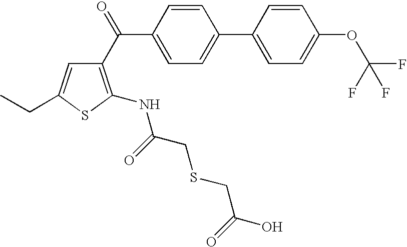

The title compound was made by an analogous procedure to Example 38, using 4-(trifluoromethoxy)benzeneboronic acid in the final step.

¹H NMR (400 MHz, CDCl₃) δ=12.54 (1H, bs), 7.75 (2H, d, J=8 Hz), 7.60 (4H, dd, J=8, 8 Hz), 7.26 (2H, d, J=8 Hz), 6.78 (1H, s), 3.61 (2H, s), 3.38 (2H, s), 2.70 (2H, q, J=8 Hz), 1.23 (3H, t, J=8 Hz).

LCMS (Method A): R_{T}=12.57 min. m/z=524 (ES+, M+H), 522 (ES−, M−H)

### Example 40

### {[5-Ethyl-3-(4′-fluoro-biphenyl-4-carbonyl)-thiophen-2-ylcarbamoyl]-methylsulfanyl}-acetic acid

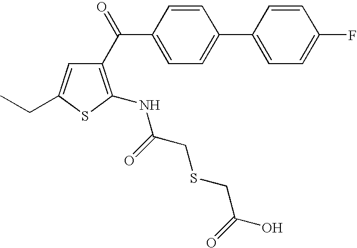

The title compound was made by an analogous procedure to Example 38, using 4-fluorobenzeneboronic acid in the final step.

¹H NMR (400 MHz, CDCl₃) δ=12.54 (1H, bs), 7.74 (2H, d, J=8 Hz), 7.59 (2H, d, J=8 Hz), 7.54 (2H, dd, J=9, 5 Hz), 7.10 (2H, dd, J=9, 9 Hz), 6.79 (1H, s), 3.61 (2H, s), 3.38 (2H, s), 2.70 (2H, q, J=7 Hz), 1.23 (3H, t, J=7 Hz).

LCMS (Method B): R_{T}=12.31 min. m/z=458 (ES+, M+H), 456 (ES−, M−H)

### Example 41

### {[5-Ethyl-3-(4-pyrimidin-5-yl-benzoyl)-thiophen-2-ylcarbamoyl]-methylsulfanyl}-acetic acid

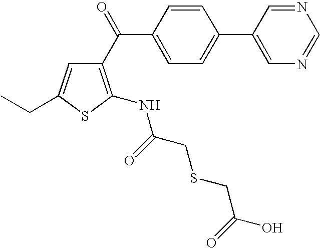

The title compound was made by an analogous procedure to Example 38, using 4-pyrimidineboronic acid in the final step.

¹H NMR (400 MHz, d6-DMSO) δ=12.26 (1H, bs), 9.26 (3H, bs), 8.02 (2H, d, J=8 Hz), 7.86 (2H, d, J=8 Hz), 6.86 (1H, s), 3.74 (2H, s), 3.42 (2H, s), 2.75 (2H, q, J=8 Hz), 1.22 (3H, t, J=8 Hz).

LCMS (Method A): R_{T}=9.25 min. m/z=442 (ES+, M+H), 440 (ES−, M−H)

### Example 42

### ({5-Ethyl-3-[4-(1-methyl-1H-pyrazol-4-yl)-benzoyl]-thiophen-2-ylcarbamoyl}-methylsulfanyl)-acetic acid

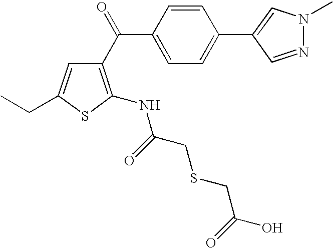

The title compound was made by an analogous procedure to Example 38, using 1-methyl-1H-pyrazol-4-boronic acid in the final step.

¹H NMR (400 MHz, d6-DMSO) δ=12.22 (1H, bs), 8.29 (1H, bs), 7.99 (1H, bs), 7.74 (4H, bs), 3.89 (3H, bs), 3.72 (2H, bs), 3.41 (2H, bs), 2.75 (2H, q, J=7 Hz), 1.22 (3H, t, J=7 Hz).

LCMS (Method A): R_{T}=9.67 min. m/z=444 (ES+, M+H), 442 (ES−, M−H)

### Example 43

### {[3-(3-Bromo-benzoyl)-5-ethyl-thiophen-2-ylcarbamoyl]-methylsulfanyl}-acetic acid

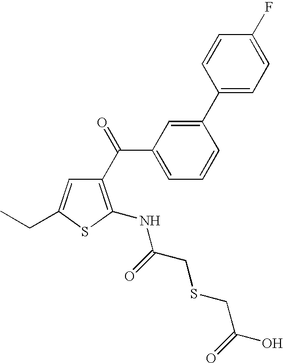

The title compound was made by an analogous procedure to Example 38, using 4-pyrimidineboronic acid and Example 34 in the final step.

¹H NMR (400 MHz, d6-DMSO) δ=12.29 (1H, bs), 7.93-7.90 (2H, m), 7.82-7.76 (2H, m), 7.72-7.63 (2H, m), 7.34 (2H, dd, J=9, 9 Hz), 6.87 (1H, s), 3.74 (2H, s), 3.41 (2H, s), 2.75 (2H, q), J 6), 1.21 (3H, t, J=6 Hz).

LCMS (Method A): R_{T}=11.60 min. m/z=458 (ES+, M+H) 456 (ES−, M−H)

### Example 44

### {[5-Ethyl-3-(4′-trifluoromethoxy-biphenyl-3-carbonyl)-thiophen-2-ylcarbamoyl]-methylsulfanyl}-acetic acid

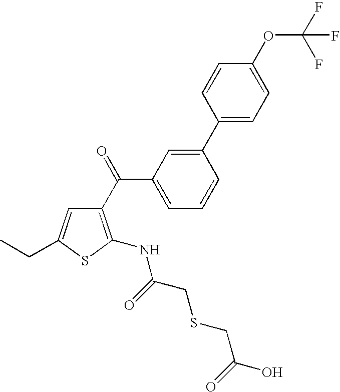

The title compound was made by an analogous procedure to Example 38, using 4-(trifluoromethoxy)benzeneboronic acid and Example 34 in the final step.

¹H NMR (400 MHz, d6-DMSO) δ=12.30 (1H, bs), 7.98-7.93 (2H, m), 7.90-7.85 (2H, m), 7.75-7.65 (2H, m), 7.49 (2H, d, J=8 Hz), 6.87 (1H, s), 3.74 (2H, s), 3.41 (2H, s), 2.75 (2H, q, J=8 Hz), 1.21 (3H, t, J=8 Hz).

LCMS (Method A): R_{T}=12.48 min. m/z=524 (ES+, M+H), 522 (ES−, M−H)

### Example 45

### {[5-Ethyl-3-(4-trifluoromethylbenzoyl)-thiophen-2-ylcarbamoyl]-methylsulfanyl}-acetic acid

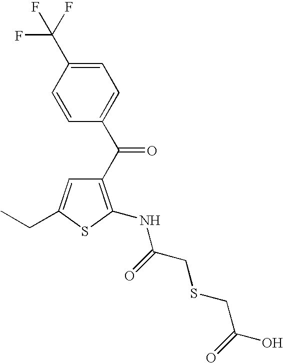

The title compound was prepared from [(4-trifluoromethyl)benzoyl]acetonitrile by an analogous procedure to Example 1.

¹H NMR (400 MHz, DMSO-d6) δ=12.23 (1H, s), 7.94 (2H, d, J=8 Hz), 7.90 (2H, d, J=8 Hz), 6.80 (1H, s), 3.75 (2H, s), 3.43 (2H, s), 2.74 (2H, qd, J=8, 1 Hz), 1.20 (3H, t, J=8 Hz).

LCMS (Method B): R_{T}=10.84 min. m/z=432 (ES+, M+H), 430 (ES−, M−H)

### Example 46

### {[5-Ethyl-3-(naphthalene-1-carbonyl)-thiophen-2-ylcarbamoyl]-methylsulfanyl}-acetic acid

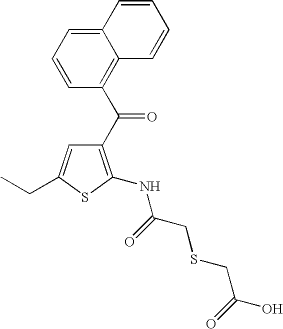

The title compound was prepared from 1-naphthylacetonitrile by an analogous procedure to Example 1.

¹H NMR (400 MHz, DMSO-d6) δ=12.53 (1H, bs), 8.12 (1H, t, J=9 Hz), 8.05 (1H, d, J=7 Hz), 7.91 (1H, d, J=8 Hz), 7.72-7.54 (4H, m), 6.40 (1H, s), 3.76 (2H, s), 3.31 (2H, s), 2.63 (2H, q, J=7 Hz), 1.10 (3H, t, J=7 Hz).

LCMS (Method A): R_{T}=11.54 min. m/z=414 (ES+, M+H), 412 (ES−, M−H)

### Example 47

### {[5-Ethyl-3-(naphthalene-2-carbonyl)-thiophen-2-ylcarbamoyl]-methylsulfanyl}-acetic acid

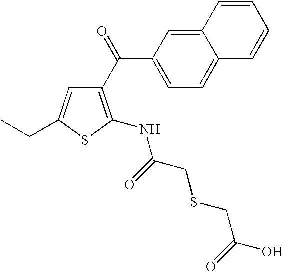

The title compound was prepared from 2-naphthylacetonitrile by an analogous procedure to Example 1.

¹H NMR (400 MHz, CDCl₃) δ=12.58 (1H, bs), 8.17 (1H, bs), 7.90-7.83 (3H, m), 7.74 (1H, dd, J=8, 2 Hz), 7.57-7.48 (2H, m), 6.80 (1H, s), 3.61 (2H, s), 3.38 (2H, s), 2.70 (2H, q, J=7 Hz), 1.22 (3H, t, J=7 Hz).

LCMS (Method B): R_{T}=11.05 min. m/z=414 (ES+, M+H), 412 (ES−, M−H)

### Example 48

### {[5-Ethyl-3-(3-methoxybenzoyl)-thiophen-2-ylcarbamoyl]-methylsulfanyl}-acetic acid

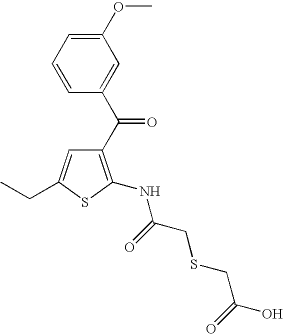

The title compound was prepared from (3-methoxybenzoyl)acetonitrile by an analogous procedure to Example 1.

¹H NMR (400 MHz, CDCl₃) δ=12.52 (1H, bs), 7.32 (1H, t, J=8 Hz), 7.22 (1H, dt, J=7, 1 Hz), 7.17 (1H, dd, J=3, 2 Hz), 7.03 (1H, ddd, J=8, 3, 1 Hz), 6.75 (1H, t, J=1 Hz), 3.79 (3H, s), 3.59 (2H, s,), 3.35 (2H, s), 2.68 (2H, qd, J=8, 1 Hz), 1.21 (3H, t, J=8 Hz)

LCMS (Method A): R_{T}=10.65 min. m/z=394 (ES+, M+H), 392 (ES−, M−H)

### Example 49

### {[3-(3,4-Dimethoxybenzoyl)-5-ethyl-thiophen-2-ylcarbamoyl]-methylsulfanyl}-acetic acid

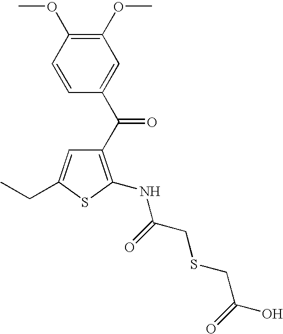

The title compound was prepared from (3,4-dimethoxybenzoyl)acetonitrile by an analogous procedure to Example 1.

¹H NMR (400 MHz, CDCl₃) δ=12.45 (1H, s), 7.33 (1H, dd, J=8, 2 Hz), 7.27 (1H, d, J=2 Hz), 6.86 (1H, d, J=8 Hz), 6.80 (1H, s), 3.90 (3H, s), 3.87 (3H, s), 3.58 (2H, s), 3.35 (2H, s), 2.69 (2H, q, J=8 Hz), 1.22 (3H, t, J=8 Hz).

LCMS (Method A): R_{T}=9.87 min. m/z=424 (ES+, M+H), 422 (ES−, M−H)

### Example 50

### {[3-(4-tert-Butyl-benzoyl)-5-ethyl-thiophen-2-ylcarbamoyl]-methylsulfanyl}-acetic acid

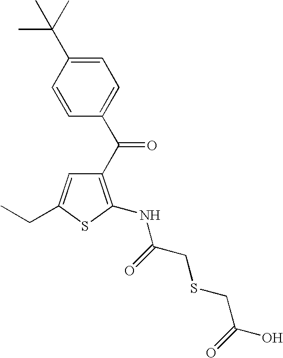

The title compound was prepared from (4-t-butylbenzoyl)acetonitrile by an analogous procedure to Example 1.

¹H NMR (400 MHz, CDCl₃) δ=12.54 (1H, bs), 7.61 (2H, d, J=9 Hz), 7.43 (2H, d, J=9 Hz), 6.78 (1H, s), 3.60 (2H, s), 3.36 (2H, s), 2.68 (2H, qd, J=8, 1 Hz), 1.30 (9H, s), 1.22 (3H, t, J=8 Hz).

LCMS (Method A): R_{T}=12.25 min m/z=420 (ES+, M+H), 418 (ES−, M−H)

### Example 51

### {[3-(3,4-Dimethyl-benzoyl)-5-ethyl-thiophen-2-ylcarbamoyl]-methylsulfanyl}-acetic acid

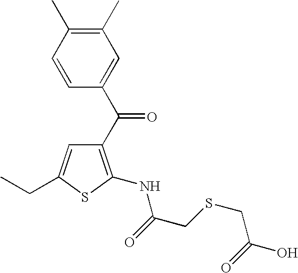

The title compound was prepared from (3,4-dimethylbenzoyl)acetonitrile by an analogous procedure to Example 1.

¹H NMR (400 MHz, DMSO-d6) δ=7.48 (1H, s), 7.43 (1H, d, J=8.0 Hz), 7.29 (1H, d, J=8.0 Hz), 6.80 (1H, s), 3.68 (2H, s), 3.32 (2H, s), 2.72 (2H, q, J=7.5 Hz), 2.30 (3H, s), 2.29 (3H, s), 1.20 (3H, t, J=7.5 Hz).

LCMS (Method A): R_{T}=11.76 min. m/z=392 (ES+, M+H), 390 (ES−, M−H)

### Example 52

### 4-[3-(3,4-Dimethyl-benzoyl)-5-ethyl-thiophen-2-ylcarbamoyl]-3,3-dimethyl-butyric acid

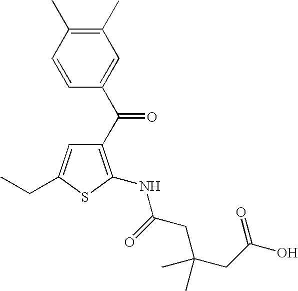

The title compound was made by an analogous procedure to Example 51, using 3,3-dimethylglutaric anhydride in the final step.

¹H NMR (400 MHz, CDCl₃) δ=7.51 (1H, s), 7.46 (1H, d, J=7.7 Hz), 7.24 (1H, d, J=7.7 Hz), 6.84 (1H, s), 2.76 (2H, q, J=7.5 Hz), 2.62 (2H, s), 2.49 (2H, s), 2.35 (3H, s), 2.34 (3H, s), 1.29 (3H, t, J=7.5 Hz), 1.20 (6H, s).

LCMS (Method A): R_{T}=12.92 min. m/z=402 (ES+, M+H), 400 (ES−, M−H)

### Example 53

### (1-{[3-(3,4-Dimethyl-benzoyl)-5-ethyl-thiophen-2-ylcarbamoyl]-methyl}-cyclopentyl)-acetic acid

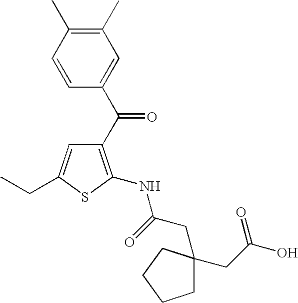

The title compound was made by an analogous procedure to Example 51, using 8-oxa-spiro[4.5]decane-7,9-dione in the final step.

¹H NMR (400 MHz, CDCl₃) δ=12.16 (1H, s), 7.48 (1H, s), 7.43 (1H, d, J=8.2 Hz), 7.21 (1H, d, J=8.2 Hz), 6.80 (1H, s), 2.74 (1H, q, J=7.5 Hz), 2.71 (2H, s), 2.53 (2H, s), 2.33 (3H, s), 2.31 (3H, s), 1.73-1.65 (8H, m), 1.27 (3H, t, J=7.5 Hz).

LCMS (Method A): R_{T}=13.60 min. m/z=428 (ES+, M+H), 426 (ES−, M−H)

### Example 54

### 2-{[3-(3,4-Dimethyl-benzoyl)-5-ethyl-thiophen-2-ylcarbamoyl]-methylsulfanyl}-2-methyl-propionic acid

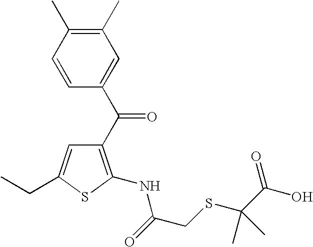

The title compound was made by an analogous procedure to Example 51, using 3,3-dimethyl-[1,4]oxathiane-2,6-dione in the final step.

¹H NMR (400 MHz, CDCl₃) δ=7.52 (1H, s), 7.46 (1H, d, J=7.7 Hz), 7.23 (1H, d, J=7.7 Hz), 6.79 (1H, s), 3.68 (2H, s), 2.73 (2H, q, J=7.3 Hz), 2.34 (3H, s), 2.33 (3H, s), 1.26 (3H, t, J=7.3 Hz).

LCMS (Method A): R_{T}=12.65 min. m/z=420 (ES+, M+H), 418 (ES−, M−H)

### Example 55

### {[5-Ethyl-3-(6-methoxy-pyridine-3-carbonyl)-thiophen-2-ylcarbamoyl]-methylsulfanyl}-acetic acid

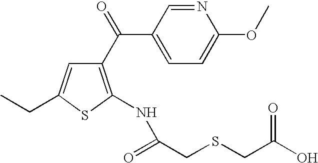

The title compound was prepared from 3-(6-methoxy-pyridin-3-yl)-3-oxo-propionitrile by an analogous procedure to Example 1.

¹H NMR (400 MHz, DMSO-d6) δ=12.41 (1H, bs), 8.56 (1H, dd, J=2.4, 0.4 Hz), 8.05 (1H, dd, J=8.7, 2.5 Hz), 6.97 (1H, dd, J=8.6, 0.4 Hz), 6.88 (1H, s), 3.96 (3H, s), 3.65 (2H, s), 3.15 (2H, s), 2.74 (2H, q, J=7.2 Hz), 1.22 (3H, t, J=7.5 Hz)

LCMS (Method A): R_{T}=10.12 min. m/z=395 (ES+, M+H, 100), 393 (ES−, M−H, 70), 349 (ES−, 75), 261 (ES−, 100)

### Example 56

### (1-{[5-Ethyl-3-(6-methoxy-pyridine-3-carbonyl)-thiophen-2-ylcarbamoyl]-methyl}-cyclopentyl)-acetic acid

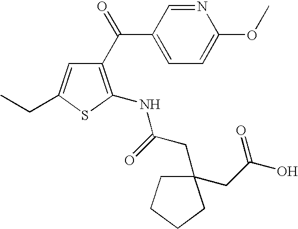

The title compound was prepared by an analogous procedure to Example 55, using 8-oxa-spiro[4.5]decane-7,9-dione in the final step.

¹H NMR (400 MHz, DMSO-d6) δ=12.18 (1H, bs), 11.69 (1H, bs), 8.56 (1H, dd, J=2.5, 0.6 Hz), 8.04 (1H, dd, J=8.7, 2.5 Hz), 6.98 (1H, dd, J=8.6, 0.6 Hz), 6.87 (1H, t, J=1.0 Hz), 3.96 (3H, s), 2.74 (2H, qd, J=7.5, 1.1 Hz), 2.73 (2H, m), 2.39 (2H, s), 1.62 (8H, m), 1.22 (3H, t, J=7.5 Hz)

LCMS (Method A): R_{T}=12.23 min. m/z=431 (ES+, M+H, 25), 263 (ES+, 100), 429 (ES−, M−H, 90), 261 (ES−, 100)

### Example 57

### {[5-Ethyl-3-(6-trifluoromethyl-pyridine-3-carbonyl)-thiophen-2-ylcarbamoyl]-methylsulfanyl}-acetic acid

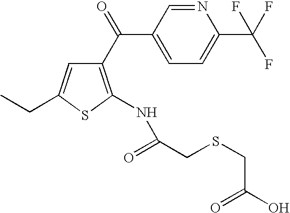

The title compound was prepared from 3-oxo-3-(6-trifluoromethyl-pyridin-3-yl)-propionitrile by an analogous procedure to Example 1.

¹H NMR (400 MHz, DMSO-d6) δ=12.6 (1H, bs), 9.01 (1H, s), 8.35 (1H, dd, J=8.1, 1.5 Hz), 8.08 (1H, d, J=8.1 Hz), 6.84 (1H, s), 3.64 (2H, s), 3.11 (2H, s), 2.73 (2H, q, J=7.4 Hz), 1.21 (3H, t, J=7.5 Hz)

LCMS (Method A) R_{T}=10.65 min. m/z=433 (ES+, M+H, 100), 301 (ES+, 100), 431 (ES−, M−H, 80), 299 (ES−, 100)

### Example 58

### {[5-Ethyl-3-(6-isopropoxy-pyridine-3-carbonyl)-thiophen-2-ylcarbamoyl]-methylsulfanyl}-acetic acid

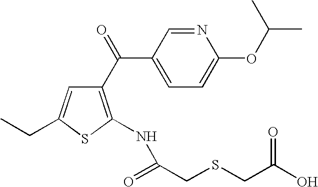

### {[5-Ethyl-3-(6-isopropoxy-pyridine-3-carbonyl)-thiophen-2-ylcarbamoyl]-methylsulfanyl}-acetic acid

### Step 1: Isopropyl 6-isopropoxynicotinate

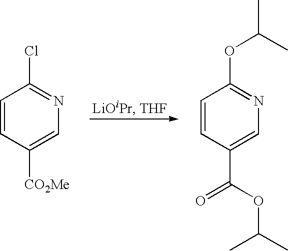

6-Chloronicotinyl chloride (704 mg, 4 mmol) was dissolved in a 2 M solution of lithium isopropoxide in THF (8 mL, 16 mmol) and the red solution microwave irradiated at 130° C. for 30 minutes. After cooling the solution was diluted with water and extracted twice with dichloromethane and twice with diethyl ether. The combined organic extracts were evaporated to dryness and the residue dissolved in diethyl ether. The ether solution was washed with water and brine, dried over sodium sulphate, filtered and evaporated to dryness. The crude red oil was purified by column chromatography (silica, 10% diethyl ether in petroleum ether) providing the desired product as a yellow oil (508 mg, 57% yield).

The remaining synthetic steps were performed by an analogous procedure to Example 1.

¹H NMR (400 MHz, DMSO-d6) δ=12.39 (1H, bs), 8.54 (1H, dd, J=2.5, 0.6 Hz), 8.02 (1H, dd, J=8.6, 2.5 Hz), 6.88 (2H, m), 5.36 (1H, septet, J=6.2 Hz), 3.65 (2H, s), 3.17 (2H, s), 2.75 (2H, qd, J=7.4, 0.7 Hz), 1.34 (6H, d, J=6.2 Hz), 1.22 (3H, t, J=7.5 Hz)

LCMS (Method A) R_{T}=11.65 min. m/z=423 (ES+, M+H), 421 (ES−, M−H)

### Example 59

### {[3-(5-Chloro-6-isopropoxy-pyridine-3-carbonyl)-5-ethyl-thiophen-2-ylcarbamoyl]-methylsulfanyl}-acetic acid

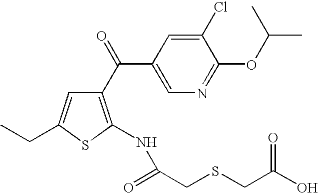

The title compound was prepared from 5,6-dichloronicotinoyl chloride by an analogous procedure to Example 58.

¹H NMR (400 MHz, DMSO-d6) δ=12.47 (1H, bs), 8.46 (1H, d, J=2.2 Hz), 8.13 (1H, d, J=2.2 Hz), 6.91 (1H, s), 5.42 (1H, septet, J=6.3 Hz), 3.63 (2H, s), 2.74 (2H, qd, J=7.5, 0.8 Hz), 1.37 (6H, d, J=6.2 Hz), 1.22 (3H, t, J=7.5 Hz)

LCMS (Method A) R_{T}=12.51 min. m/z=457/459 (ES+, M+H), 455/457 (ES−, M−H)

### Example 60

### {[5-Ethyl-3-(6-phenoxy-pyridine-3-carbonyl)-thiophen-2-ylcarbamoyl]-methylsulfanyl}-acetic acid

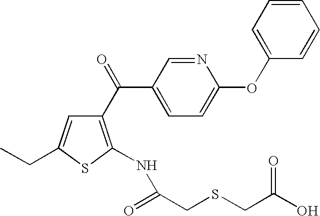

### Step 1: Methyl 6-phenoxynicotinate

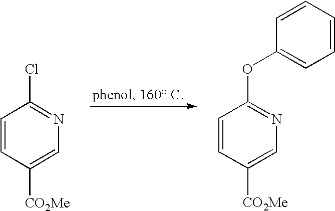

Methyl 6-chloronicotinate (1.20 g, 7 mmol) was dissolved in molten phenol (10 g, 106 mmol) and the solution heated at 160° C. for 19 h. After cooling the mixture was diluted with 1M aq. NaOH (100 mL) and extracted with ethyl acetate (2×100 mL). The combined organic phases were washed with 1M aq. NaOH (3×100 mL) and brine (100 mL), dried over sodium sulphate, filtered and evaporated to dryness. The desired product was obtained as a white solid (1.07 g, 67% yield), contaminated with 10% of the phenyl ester.

The remaining synthetic steps were performed by an analogous procedure to Example 1.

¹H NMR (400 MHz, DMSO-d6) δ=12.43 (1H, bs), 8.49 (1H, dd, J=2.5, 0.6 Hz), 8.18 (1H, dd, J=8.6, 2.5 Hz), 7.47 (2H, m), 7.28 (1H, m), 7.22 (2H, m), 7.16 (1H, dd, J=8.6, 0.5 Hz), 6.88 (1H, s), 3.65 (2H, s), 3.16 (2H, s), 2.73 (2H, qd, J=7.5, 0.7 Hz), 1.21 (3H, t, J=7.5 Hz)

LCMS (Method A) R_{T}=11.33 min. m/z=457 (ES+, M+H), 455 (ES−, M−H)

### Example 61

### {[3-(4-Methoxy-benzoyl)-5-methyl-thiophen-2-ylcarbamoyl]-methylsulfanyl}-acetic acid

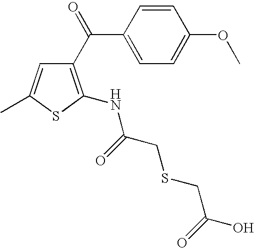

The title compound was prepared by an analogous procedure to Example 1, using (4-methoxybenzoyl)acetonitrile and propionaldehyde in Step 2.

¹H NMR (400 MHz, DMSO-d6) δ=12.16 (1H, bs), 7.74 (2H, d, J=9 Hz), 7.10 (2H, d, J=9 Hz), 6.86 (1H, s), 3.86 (3H, s), 3.70 (2H, s), 3.40 (2H, s).

LCMS (Method A) R_{T}=9.92 min. m/z=380 (ES+, M+H), 378 (ES−, M−H)

### Example 62

### {[5-Isopropyl-3-(4-methoxy-benzoyl)-thiophen-2-ylcarbamoyl]-methylsulfanyl}-acetic acid

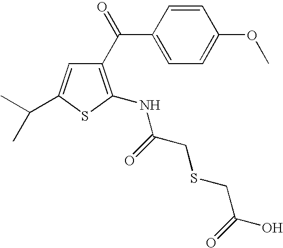

The title compound was prepared by an analogous procedure to Example 61, using isovaleraldehyde in Step 2.

¹H NMR (400 MHz, DMSO-d6) δ=12.16 (1H, s), 7.74 (2H, d, J=9 Hz), 7.11 (2H, d, J=9 Hz), 6.85 (1H, s), 3.87 (3H, s), 3.70 (2H, s), 3.40 (2H, s), 3.14-3.07 (1H, m), 1.26 (6H, d, J=7 Hz).

LCMS (Method A) R_{T}=11.04 min. m/z=408 (ES+, M+H), 406 (ES−, M−H)

### Example 63

### {[3-(4-Methoxybenzoyl)-5-propyl-thiophen-2-ylcarbamoyl]-methylsulfanyl}-acetic acid

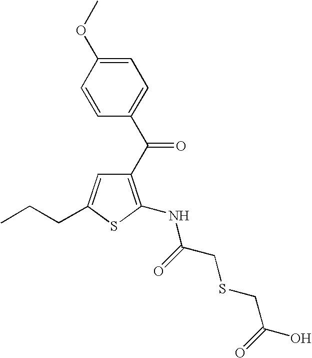

The title compound was prepared by an analogous procedure to Example 61, using valeraldehyde in Step 2.

¹H NMR (400 MHz, CDCl₃) δ=12.49 (1H, s), 7.69 (2H, d, J=9 Hz), 6.91 (2H, d, J=9 Hz), 6.76 (1H, s), 3.82 (3H, s), 3.60 (2H, s), 3.36 (2H, s), 2.63 (2H, t, J=7 Hz), 1.66-1.56 (2H, m), 0.89 (3H, t, J=7 Hz).

LCMS (Method A) R_{T}=11.18 min. m/z=408 (ES+, M+H), 406 (ES−, M−H)

### Example 64

### {[5-Cyclopropyl-3-(4-fluoro-benzoyl)-thiophen-2-ylcarbamoyl]-methylsulfanyl}-acetic acid

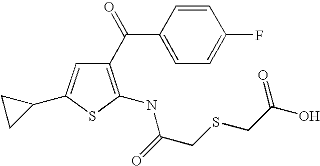

The title compound was prepared by an analogous procedure to Example 61, using cyclopropylacetaldehyde in Step 2.

¹H NMR (400 MHz, DMSO-d6) δ=12.38 (1H, bs), 7.79 (2H, dd, J=8.8, 5.5 Hz), 7.37 (2H, t, J=8.9 Hz), 6.76 (1H, d, J=0.8 Hz), 3.64 (2H, s), 3.13 (2H, s), 2.06 (1H, m), 0.94 (2H, ddd, J=8.3, 6.6, 4.3 Hz), 0.66 (2H, m).

LCMS (Method A) R_{T}=10.88 min. m/z=394 (ES+, M+H), 392 (ES−, M−H)

### Example 65

### {[5-Chloro-3-(6-methoxy-pyridine-3-carbonyl)-thiophen-2-ylcarbamoyl]-methylsulfanyl}-acetic acid

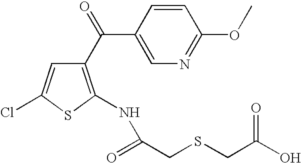

### Step 1: (2-Amino-thiophen-3-yl)-(6-methoxy-pyridin-3-yl)-methanone

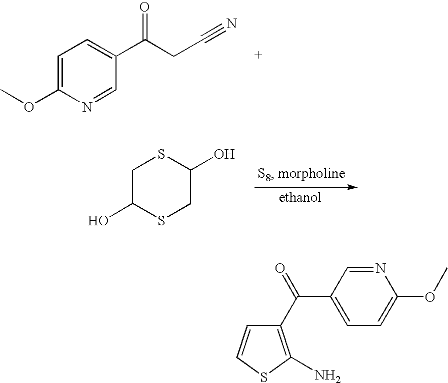

A suspension of 3-(6-methoxy-pyridin-3-yl)-3-oxo-propionitrile (1.0 g, 5.7 mmol, 1 eq.) and 2,5-dihydroxy-1,4-dithiane (433 mg, 2.85 mmol, 0.5 eq.) in morpholine (1.2 mL) and ethanol (2.4 mL) is microwave irradiated at 100° C. for 10 minutes. After cooling the solvent is removed in vacuo and the residue purified by column chromatography (1:1 diethyl ether/petroleum ether) providing the title compound as a yellow solid (948 mg, 71% yield).

This reaction can also be performed by conventional heating, at 80° C. for around 1 hour.

### Step 2: (2-Amino-5-chloro-thiophen-3-yl)-(6-methoxy-pyridin-3-yl)-methanone

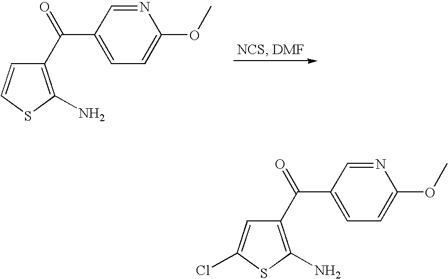

A solution of (2-amino-thiophen-3-yl)-(6-methoxy-pyridin-3-yl)-methanone (234 mg, 1 mmol) in dimethylformamide (5 mL) is treated with N-chlorosuccinimide (160 mg, 1.2 mmol) and the solution stirred at room temperature for 1.5 h. The solution is diluted with ethyl acetate, washed twice with brine and evaporated to dryness. The crude material is purified by column chromatography (silica, 1:1 ethyl acetate/petroleum ether) providing the desired chlorothiophene as a dark yellow solid (157 mg, 59% yield).

The final step, reaction of the aminothiophene with thiodiglycolic anhydride was performed as for Example 1.

¹H NMR (400 MHz, DMSO-d6) δ=8.56 (1H, d, J=2.3 Hz), 8.05 (1H, dd, J=8.7, 2.5 Hz), 7.18 (1H, s), 6.95 (1H, d, J=8.6 Hz), 3.95 (3H, s), 3.60 (2H, bs), 3.11 (2H, bs)

LCMS (Method A) R_{T}=10.21 min. m/z=401/403 (ES+, M+H), 399/401 (ES−, M−H)

### Example 66

### {[5-Chloro-3-(4-methoxy-benzoyl)-thiophen-2-ylcarbamoyl]-methylsulfanyl}-acetic acid

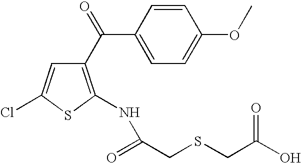

The title compound was prepared by an analogous procedure to Example 65, starting from (4-methoxybenzoyl)acetonitrile.

¹H NMR (400 MHz, DMSO-d6) δ=7.75 (2H, d, J=8.8 Hz), 7.13 (1H, s), 7.09 (2H, d, J=8.8 Hz), 3.86 (3H, s), 3.63 (2H, s), 3.11 (2H, s)

LCMS (Method A) R_{T}=10.69 min. m/z=400/402 (ES+, M+H, 55), 268/270 (ES+, 100), 398/400 (ES−, M−H, 20), 354/356 (ES−, 15), 230 (ES−, 100)

### Example 67

### {[5-Chloro-3-(4-trifluoromethoxy-benzoyl)-thiophen-2-ylcarbamoyl]-methylsulfanyl}-acetic acid

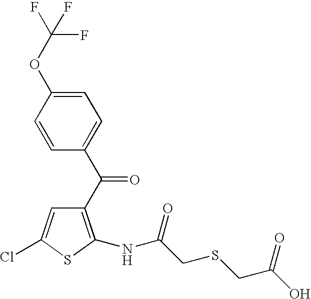

The title compound was prepared by an analogous procedure to Example 65, starting from [(4-trifluoromethoxy)benzoyl]acetonitrile.

¹H NMR (400 MHz, CDCl₃) δ=12.63 (1H, bs), 7.77 (2H, d, J=8.8 Hz), 7.34 (2H, d, J=8.8 Hz), 6.98 (1H, s), 3.67 (2H, s), 3.42 (2H, s)

LCMS (Method A) R_{T}=8.66 min. m/z=4561454 (ES+, M+H), 454/452 (ES−, M−H)

### Example 68

### {[3-(4-Methoxy-benzoyl)-thiophen-2-ylcarbamoyl]-methylsulfanyl}-acetic acid

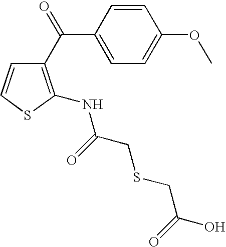

The title compound was prepared by an analogous procedure to Example 65, but bypassing the chlorination procedure of Step 2.

¹H NMR (400 MHz, DMSO-d6) δ=12.20 (1H, bs), 7.76 (2H, d, J=9 Hz), 7.16 (1H, d, J=6 Hz), 7.13-7.05 (3H, m), 3.86 (3H, s), 3.73 (2H, s), 3.42 (2H, s).

LCMS (Method A) R_{T}=9.32 min. m/z=366 (ES+, M+H), 364 (ES−, M−H)

### Example 69

### 5-[5-Ethyl-3-(4-methoxy-benzoyl)-thiophen-2-ylcarbamoyl]-pentanoic acid

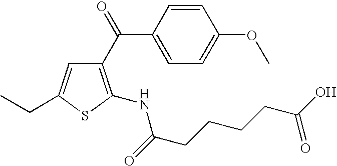

### General Procedures for Introduction of Side Chains

### Method A

Acylation of a 2-Aminothiophene with a Chloro-Acid Chloride.

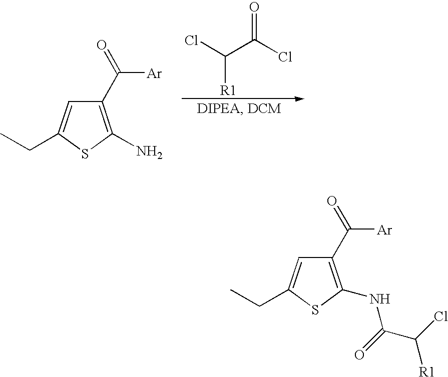

A solution of the aminothiophene (1 eq.) and diisopropylamine (1.5 eq.) in dichloromethane (0.11) is cooled to 0° C. and treated with the appropriate chloroacyl chloride (1.5 eq.). After 10 minutes at 0° C. the solution is allowed to warm to room temperature and stirred until reaction is complete (1-3 hours, depending on substituents). The solution is diluted with dichloromethane and washed with water twice and brine. The combined organic phases are dried over sodium sulphate, filtered and concentrated to dryness in vacuo. Purification is by column chromatography (30% diethyl ether/petroleum ether) or trituration in diethyl ether/petroleum ether.

### Method B

Reaction of the Chloroamide with a Thiol Ester or Benzenethiol.

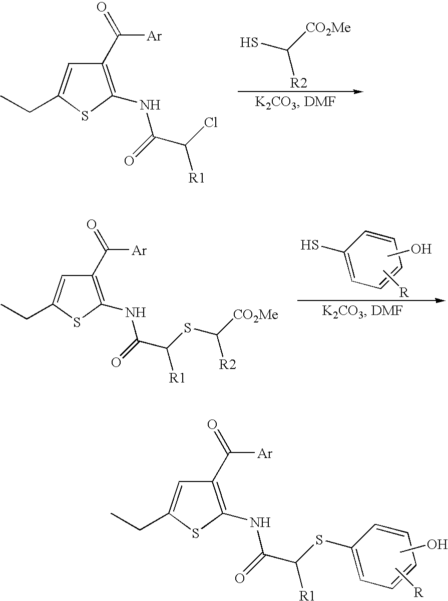

A solution of the chloroamide (1 eq.) and potassium carbonate (2 eq.) in DMF (0.1 M) is treated with the thiol ester (2 eq.) or mercaptophenol (2 eq.) at room temperature. After complete reaction is observed, the mixture is dissolved in water and extracted twice with ethyl acetate. The combined organic phases are washed with water 3 times and brine, dried over sodium sulphate, filtered and concentrated to dryness in vacuo. Purification is achieved by column chromatography on silica gel (30-50% diethyl ether/petroleum ether).

### Method C

### Hydrolysis of the Methyl Ester.

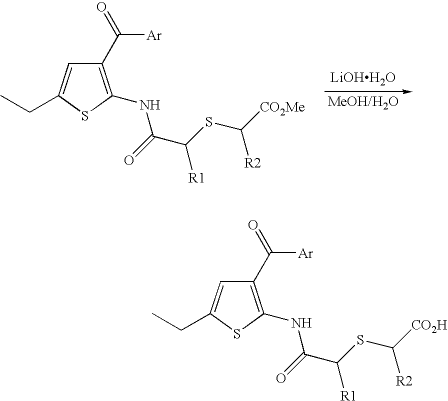

A solution of the methyl ester (1 eq.) in methanol (10 mL/mmol) and water (10 mL/mmol) is treated with lithium hydroxide monohydrate (2 eq.) and stirred at room temperature for 1-3 hours. Prolonged reaction times should be avoided to minimise hydrolysis of the amide. On complete reaction, water is added and the pH adjusted to pH 4-5 with 1M hydrochloric acid. The solution is extracted twice with ethyl acetate and the combined extracts dried over sodium sulphate, filtered and concentrated to dryness in vacuo. Purification is by column chromatography (1:1 ethyl acetate/petroleum ether) then trituration in diethyl ether.

### Method D

Reaction of the Chloroamide with an Aminoester.

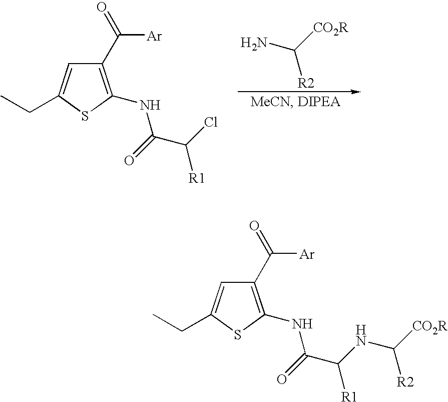

A solution of the chloride (1 eq.) and aminoester (2 eq.) in acetonitrile (0.1 M) is treated with diisopropylamine (2 eq.) and the reaction heated to reflux for 1-2 days. On complete reaction, the solution is allowed to cool, dissolved in ethyl acetate and washed with saturated ammonium chloride solution and brine. The organic solution is dried over sodium sulphate, filtered and concentrated to dryness in vacuo. If necessary, purification can be achieved by trituration in diethyl ether/petroleum ether.

### Method E

### Conversion of the Tert-Butyl Ester to the Corresponding Acid.

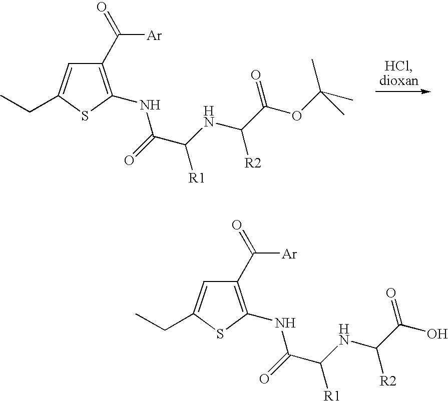

The tert-butyl ester is dissolved in 4M HCl/dioxane (1 mL/0.1 mmol) and stirred at room temperature over night. After removal of solvent under vacuum the crude solid is purified by trituration in diethyl ether/petroleum ether.

If the crude material is a gum, it can be purified as follows. Dissolve in ethyl acetate, wash with water and brine, dry over sodium sulphate, filter and concentrate to dryness. The resulting solid can be purified further by trituration in diethyl ether/petroleum ether.

Basic compounds prepared by this method are isolated as the corresponding hydrochloride salt.

### Method F

Reaction of a Phenol with an Alkyl Bromoacetate.

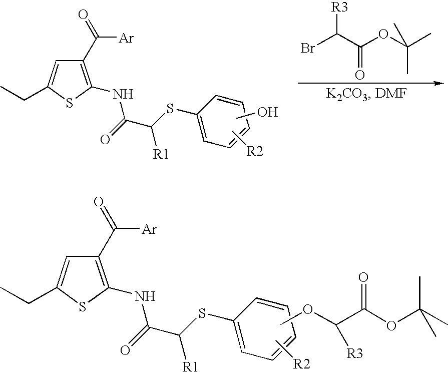

The bromoester (1.5 eq.) is added to a stirred suspension of the phenol (1 eq.) and potassium carbonate (1.5 eq.) in dimethylformamide (0.1 M) and the mixture stirred at 50° C. The reaction time is dependent on how hindered the bromide is. On complete reaction, the mixture is dissolved in ethyl acetate and washed with water three times and with brine. The organic solution is dried over sodium sulphate, filtered and evaporated to dryness in vacuo.

### Method G

### Oxidation of a Benzaldehyde to a Benzoic Acid.

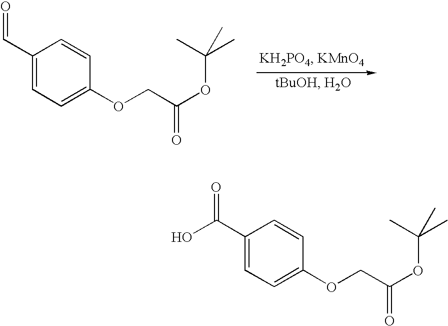

A solution of the aldehyde (2.12 mmol) in t-BuOH (3 mL) is treated with a 1.25 M aqueous solution of KH₂PO₄ (˜7 mL) until the solution is at pH 4-5. A 1M aqueous solution of KMnO₄ (3 mL) is then added and the reaction stirred at room temperature for 2 hours. The mixture is dissolved in ethyl acetate and washed with 1 M aqueous HCl three times and brine twice. The organic solution is dried over sodium sulphate, filtered and evaporated to dryness in vacuo. The crude material is used without further purification.

### Method H

### Conversion of a Benzoic Acid to the Corresponding Benzoyl Chloride.

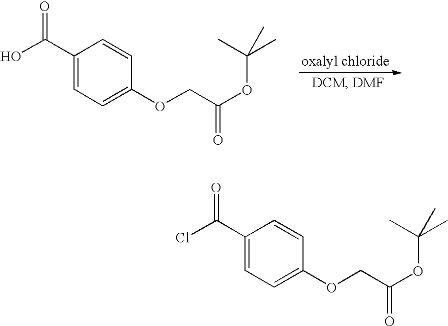

A solution of the acid (1 eq.) in dichloromethane is cooled to 0° C. under nitrogen and treated dropwise with oxalyl chloride (10 eq.) then dimethylformamide (1 drop). After 10 minutes at 0° C. the reaction is allowed to warm to room temperature and stirred for 1-2 hours. On complete conversion the solvent is removed in vacuo and the residue concentrated to dryness from dichloromethane three times to remove residual volatiles. This material is used without further purification.

### Method I

Reaction of an Aryl Bromide with an Unsaturated Ester

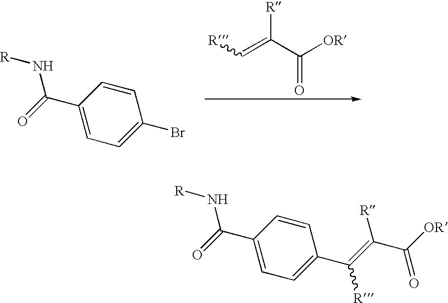

The aryl bromide (1 eq) was added to an oven dried and vacuum cooled flask and dissolved in toluene. methyl acrylate (5 eq), triethylamine (2.5 eq), tri-o-tolylphosphine (0.02 eq) and palladium acetate (0.01 eq) were added and the reaction was heated at 100° C. (20 h). The solution was diluted with ethyl acetate and filtered through celite. The organic phase is washed with 1M hydrochloric acid, saturated sodium bicarbonate and brine, dried over sodium sulphate, filtered and concentrated to dryness in vacuo. Purification is achieved by column chromatography on silica gel (15% diethyl ether/petroleum ether).

### Method J

### Reduction of a Double Bond

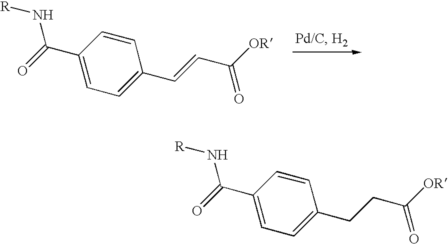

The unsaturated ester (1 eq) was dissolved in tetrahydrofuran. The flask was evacuated and filled with hydrogen three times and then the palladium on carbon was added (catalytic amount). The flask was evacuated and filled with hydrogen three times. The reaction was stirred at room temperature. After complete reaction is observed, the reaction was filtered through celite and washed through with methanol. The filtrate was concentrated to dryness in vacuo. Purification is achieved by column chromatography on silica gel (20% diethyl ether/petroleum ether).

### Method K

### Alkylation of a Heteroatom Using Sodium Hydride

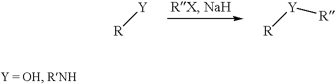

The alcohol or amine (1 eq) was dissolved in DMF and sodium hydride (1 eq) added. After 15 minutes the alkyl halide (1 eq) was added and the reaction was stirred at room temperature. After complete reaction is observed, the reaction is quenched using saturated ammonium chloride and extracted twice with ethyl acetate. The combined organic phases are washed with saturated ammonium chloride and brine, dried over sodium sulphate, filtered and concentrated to dryness in vacuo. Purification is achieved by column chromatography on silica gel (20-40% diethyl ether/petroleum ether).

### Method L

### Formation of Non-Commercially Available Thioester.

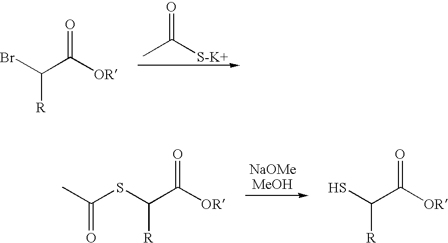

The bromide (1 eq), potassium thioacetate (1.1 eq) and charcoal (catalytic amount) were dissolved in acetone and stirred at room temperature. After complete reaction is observed, the reaction was filtered through celite and the filtrate concentrated to dryness in vacuo. The crude product was dissolved in methanol and sodium methoxide (1.3 eq) added. The reaction was stirred at room temperature. After complete reaction is observed, the reaction is concentrated to dryness in vacuo and taken crude to be reacted with a chloride using Method B.

### Method M

### Formation of Non-Commercially Available Thioester.

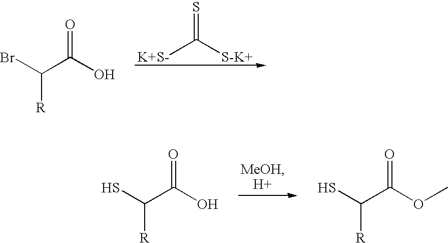

The bromide (1 eq), potassium trithiocarbonate (2 eq) were dissolved in water and heated at 70° C. for 3-4 days. After complete reaction is observed the reaction is acidified using 1M hydrochloric acid and extracted twice using ethyl acetate. The combined organic phases are washed using brine, dried over sodium sulphate, filtered and concentrated to dryness in vacuo. The compound is taken crude to be reacted with methanol and conc. HCl. After complete reaction is observed, the reaction is concentrated to dryness in vacuo and the residue dissolved saturated sodium hydrogen carbonate then extracted twice with ethyl acetate. Purification is achieved by column chromatography on silica gel (40% diethyl ether/petroleum ether).

### Example 71

### {3-[5-Ethyl-3-(4-methoxy-benzoyl)-thiophen-2-ylcarbamoyl]-propylsulfanyl}-acetic acid

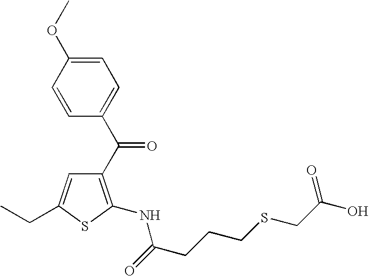

The required aminothiophene was prepared as described for Example 1 starting from (4-methoxybenzoyl)acetonitrile. The acid bearing side chain was introduced by Methods A, B and C respectively as described above.

¹H NMR (400 MHz, CDCl₃) δ=11.93 (1H, s), 7.54 (2H, d, J=8.9 Hz), 6.98 (2H, d, J=8.9 Hz), 6.80 (1H, s), 3.89 (3H, s), 3.27 (2H, s), 2.80-2.66 (6H, m), 2.09 (2H, q, J=7.1 Hz), 1.28 (3H, t, J=7.1 Hz).

LCMS (Method A): R_{T}=11.10 min. m/z=422 (ES+, M+H), 420 (ES−, M−H)

### Example 72

### {1-[5-Ethyl-3-(4-methoxy-benzoyl)-thiophen-2-ylcarbamoyl]-ethylsulfanyl}-acetic acid

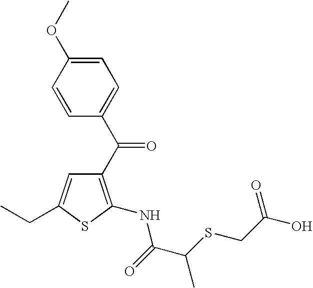

The required aminothiophene was prepared as described for Example 1 starting from (4-methoxybenzoyl)acetonitrile. The acid bearing side chain was introduced by Methods A, B and C respectively, as described above.

¹H NMR (400 MHz, CDCl₃) δ=12.52 (1H, s), 7.75 (2H, d, J=8.8 Hz), 6.98 (2H, d, J=8.8 Hz), 6.83 (1H, s), 3.92-3.87 (4H, m), 3.41 (2H, q, J=7.3 Hz), 2.76 (2H, q, J=7.5 Hz), 1.63 (3H, d, J=7.3 Hz), 1.29 (3H, t, J=7.5 Hz).

LCMS (Method A): R_{T}=11.06 min. m/z=408 (ES+, M+H), 406 (ES−, M−H)

### Example 73

### ({[5-Ethyl-3-(4-methoxy-benzoyl)-thiophen-2-ylcarbamoyl]-methyl}-methyl-amino)-acetic acid hydrochloride

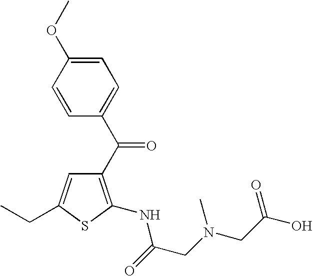

The required aminothiophene was prepared as described for Example 1 starting from (4-methoxybenzoyl)acetonitrile. The acid bearing side chain was introduced by Methods A, D and E respectively, as described above.

¹H NMR (400 MHz, DMSO-d6) δ=7.73 (2H, d, J=8.8 Hz), 7.09 (2H, d, J=8.8 Hz), 6.86 (1H, s), 3.86 (3H, s), 2.75 (2H, q, J=7.5 Hz), 2.67 (2H, s), 2.32 (2H, s), 1.22 (3H, t, J=7.5 Hz).

LCMS (Method A): R_{T}=8.18 min. m/z=391 (ES+, M+H), 389 (ES−, M−H)

### Example 74

### ({[5-Ethyl-3-(4-methoxy-benzoyl)-thiophen-2-ylcarbamoyl]-methyl}-amino)-acetic acid hydrochloride

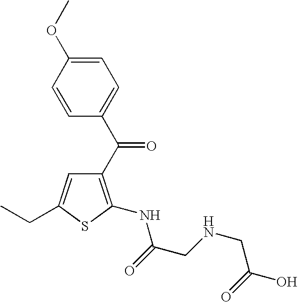

The required aminothiophene was prepared as described for Example 1 starting from (4-methoxybenzoyl)acetonitrile. The acid bearing side chain was introduced by Methods A, D and E respectively, as described above.

¹H NMR (400 MHz, MeOD-d4) δ=7.79 (2H, d, J=8.9 Hz); 7.08 (2H, d, J=8.9 Hz); 6.91 (1H, s); 4.30 (2H, s); 4.02 (2H, s); 3.92 (3H, s); 2.81 (2H, q, J=7.3 Hz); 1.32 (3H, t, J=7.3 Hz).

LCMS (Method A): R_{T}=7.17 min. m/z=377 (ES+, M+H), 375 (ES−, M−H)

### Example 75

### 2-({[5-Ethyl-3-(4-trifluoromethoxy-benzoyl)-thiophen-2-ylcarbamoyl]-methyl}-amino)-3-methyl-butyric acid

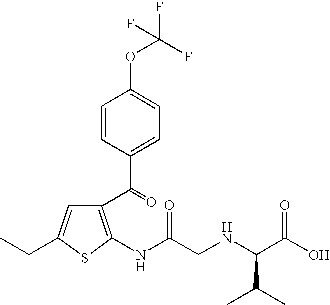

The required aminothiophene was prepared as described for Example 1. The acid bearing side chain was introduced by Methods A, D and E respectively, as described above.

¹H NMR (400 MHz, DMSO-d6) δ=7.82 (2H, d, J=8.8 Hz), 7.54 (2H, d, J=8.8 Hz), 6.80 (1H, s), 3.61 (1H, d, J=17.2 Hz), 3.23 (1H, d, J=17.2 Hz), 3.01 (1H, d, J=5.6 Hz), 2.74 (2H, q, J=7.6 Hz), 2.01 (1H, septet, J=6.4 Hz), 1.21 (3H, t, J=7.6 Hz), 1.02 (3H, d, J=6.4 Hz), 1.01 (3H, d, J=6.4 Hz).

LCMS (Method A): R_{T}=11.65 min. m/z=473 (ES+, M+H), 471 (ES−, M−H)

### Example 76

### 2-({[5-Ethyl-3-(4-trifluoromethoxy-benzoyl)-thiophen-2-ylcarbamoyl]-methyl}-amino)-2-methyl-propionic acid

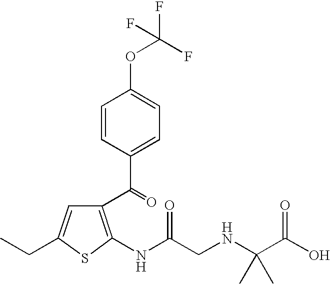

The required aminothiophene was prepared as described for Example 1. The acid bearing side chain was introduced by Methods A, D and E respectively, as described above.

¹H NMR (400 MHz, DMSO-d6) δ=7.84 (2H, d, J=8.8 Hz), 7.53 (2H, d, J=8.8 Hz), 6.80 (1H, s), 3.40 (2H, s), 2.74 (2H, q, J=8.4 Hz), 1.29 (6H, s), 1.20 (3H, t, i=7.6 Hz).

### Example 77

### 2-({[5-Ethyl-3-(4-trifluoromethoxy-benzoyl)-thiophen-2-ylcarbamoyl]-methyl}-amino)-4-methyl-pentanoic acid

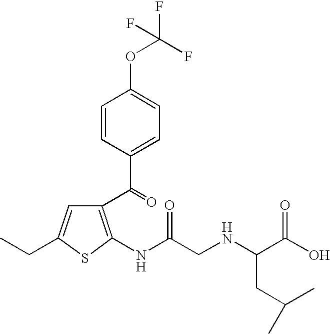

The required aminothiophene was prepared as described for Example 1. The acid bearing side chain was introduced by Methods A, D and E respectively, as described above.

¹H NMR (400 MHz, MeOD-d4) δ=7.86 (2H, d, J=8.8 Hz), 7.46 (2H, d, J=8.4 Hz), 6.82 (1H, s), 3.90 (1H, d, J=17.6 Hz), 3.61 (1H, d, J=7.6 Hz), 3.42 (1H, t, J=7.6 Hz), 2.80 (2H, q, J=7.2 Hz), 2.07 (1H, sep., J=6.4 Hz), 1.76-1.65 (2H, m), 1.30 (3H, t, 7.6 Hz), 0.98 (3H, d, J=6.4 Hz), 0.96 (3H, d, J=6.4 Hz).

LCMS (Method A): R_{T}=11.90 min. m/z=487 (ES+, M+H), 485 (ES−, M−H)

### Example 78

### (R)-2-({[5-Ethyl-3-(4-trifluoromethoxy-benzoyl)-thiophen-2-ylcarbamoyl]-methyl}-amino)-3-methyl-butyric acid

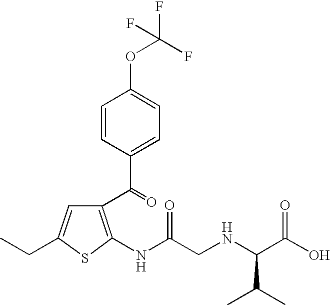

The required aminothiophene was prepared as described for Example 1. The acid bearing side chain was introduced by Methods A, D and E respectively, as described above.

¹H NMR (400 MHz, DMSO-d6) δ=7.82 (2H, d, J=8.8 Hz), 7.54 (2H, d, J=8.8 Hz), 6.80 (1H, s), 3.61 (1H, d, J=17.2 Hz), 3.23 (1H, d, J=17.2 Hz), 3.01 (1H, d, J=5.6 Hz), 2.74 (2H, q, J=7.6 Hz), 2.01 (1H, sep, J=6.4 Hz), 1.21 (3H, t, J=7.6 Hz), 1.02 (3H, d, J=6.4 Hz), 1.01 (3H, d, J=6.4 Hz).

LCMS (Method A): R_{T}=11.76 min. m/z=473 (ES+, M+H), 471 (ES−, M−H)

### Example 79

### (S)-2-({[5-Ethyl-3-(4-trifluoromethoxy-benzoyl)-thiophen-2-ylcarbamoyl]-methyl}-amino)-3-methyl-butyric acid

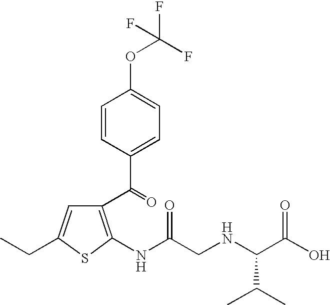

The required aminoiophene was prepared as described for Example 1. The acid bearing side chain was introduced by Methods A, D and E respectively, as described above.

¹H NMR (400 MHz, DMSO-d6) δ=7.82 (2H, d, J=8.8 Hz), 7.54 (2H, d, J=8.8 Hz), 6.80 (1H, s), 3.61 (1H, d, J=17.2 Hz), 3.23 (1H, d, J=17.2 Hz), 3.01 (1H, d, J=5.6 Hz), 2.74 (2H, q, J=7.6 Hz), 2.01 (1H, septet, J=6.4 Hz), 1.21 (3H, t, J=7.6 Hz), 1.02 (3H, d, J=6.4 Hz), 1.01 (3H, d, J=6.4 Hz).

LCMS (Method A): R_{T}=11.76 min. m/z=473 (ES+, M+H), 471 (ES−, M−H)

### Example 80

### 2-({[5-Ethyl-3-(4-trifluoromethoxy-benzoyl)-thiophen-2-ylcarbamoyl]-methyl}-amino)-butyric acid

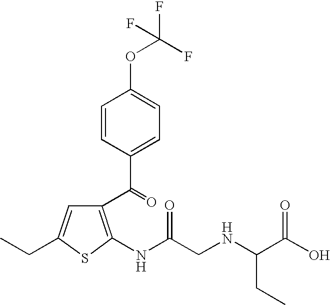

The required aminothiophene was prepared as described for Example 1. The acid bearing side chain was introduced by Methods A, D and E respectively, as described above.

¹H NMR (400 MHz, DMSO-d6) δ=7.83 (2H, d, J=8.4 Hz), 7.54 (2H, d, J=7.8 Hz), 6.80 (1H, s), 3.6 (1H, d, J=18 Hz), 3.25 (1H, d, J=18 Hz), 3.17 (1H, t, J=5.2 Hz), 2.74 (2H, q, J=5.6 Hz), 1.72 (2H, q, 7.2 Hz), 1.20 (3H, t, J=7.2 Hz), 0.99 (3H, t, J=7.6 Hz).

LCMS (Method A): R_{T}=11.81 min. m/z=459 (ES+, M+H), 457 (ES−, M−H)

### Example 81

### ({[5-Ethyl-3-(4-trifluoromethoxy-benzoyl)-thiophen-2-ylcarbamoyl]-methyl}-amino)-acetic acid

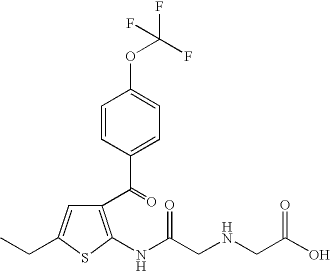

The required aminothiophene was prepared as described for Example 1. The acid bearing side chain was introduced by Methods A, D and E respectively, as described above.

¹H NMR (400 MHz, DMSO-d6) δ=7.84 (2H, d, J=8.8 Hz), 7.54 (2H, d, J=8.4 Hz), 6.81 (1H, s), 3.50 (2H, s), 3.41 (2H, s), 2.74 (2H, q, J=6.8 Hz), 1.21 (3H, t, J=7.6 Hz).

LCMS (Method A): R_{T}=8.58 min. m/z=431 (ES+, M+H), 429 (ES−, M−H)

### Example 82

### 1-({[5-Ethyl-3-(4-trifluoromethoxy-benzoyl)-thiophen-2-ylcarbamoyl]-methyl}-amino)-cyclopropanecarboxylic acid

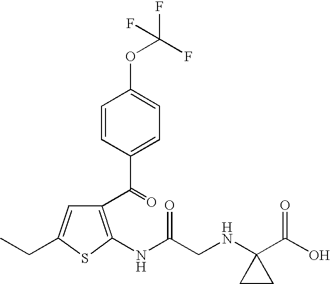

The required aminothiophene was prepared as described for Example 1. The acid bearing side chain was introduced by Methods A, D and E respectively, as described above.

¹H NMR (400 MHz, MeOD-d4) δ=7.88 (2H, d, J=8.8 Hz), 7.47 (2H, d, J=8.0 Hz), 6.86 (1H, s), 4.47 (2H, s), 2.81 (2H, q, J=7.6 Hz), 1.66-1.63 (2H, m), 1.58-1.55 (2H, m), 1.31 (3H, t, J=7.2 Hz).

LCMS (Method A): R_{T}=12.08 min. m/z=457 (ES+, M+H), 455 (ES−, M−H)

### Example 83

### 2-({[5-Ethyl-3-(4′-fluoro-biphenyl-4-carbonyl)-thiophen-2-ylcarbamoyl]-methyl}-amino)-2-methyl-propionic acid

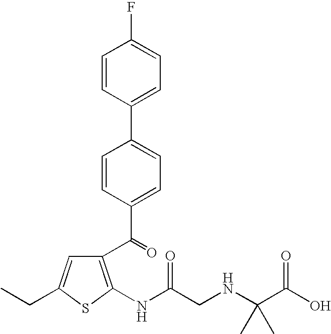

The required aminothiophene was prepared as described via the Suzuki coupling described for Example 38. The acid bearing side chain was introduced by Methods A, D and E respectively, as described above.

¹H NMR (400 MHz, d6-DMSO) δ=7.86-7.76 (6H, m), 7.36 (2H, t, J=9 Hz), 6.87 (1H, s), 3.40 (2H, s), 2.74 (2H, q, J=7 Hz), 1.30 (6H, s), 1.21 (3H, t, J=7 Hz).

LCMS (Method A): R_{T}=10.16 min. m/z=469 (ES+, M+H), 467 (ES−, M−H)

### Example 84

### 2-({[5-Ethyl-3-(4′-trifluoromethoxy-biphenyl-3-carbonyl)-thiophen-2-yl carbamoyl]-methyl}-amino)-2-methyl-propionic acid

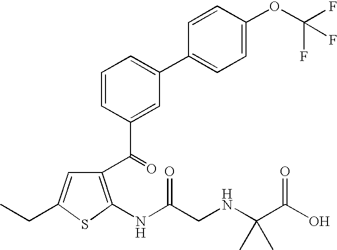

The required aminothiophene was prepared as described via the Suzuki coupling described for Example 38. The acid bearing side chain was introduced by Methods A, D and E respectively, as described above.

¹H NMR (400 MHz, d6-DMSO) δ=7.95-7.92 (2H, m), 7.87 (2H, d, J=8.8 Hz), 7.72-7.64 (2H, m), 7.5 (2H, d, J=8.4 Hz), 6.83 (1H, s), 3.41 (2H, s), 2.74 (2H, q, J=7.2 Hz), 1.30, (6H, s), 1.2 (3H, t, J=7.6 Hz).

LCMS (Method A): R_{T}=11.56 min. m/z=535 (ES+, M+H), 533 (ES−, M−H)

### Example 85

### 2-({[5-Ethyl-3-(4′-fluoro-biphenyl-3-carbonyl)-thiophen-2-ylcarbamoyl]-methyl}-amino)-2-methyl-propionic acid

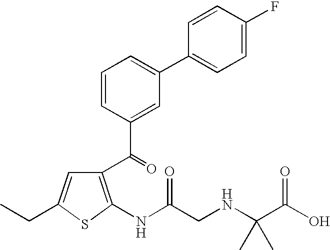

The required aminothiophene was prepared as described via the Suzuki coupling described for Example 38. The acid bearing side chain was introduced by Methods A, D and E respectively, as described above.

¹H NMR (400 MHz, d6-DMSO) δ=7.91-7.89 (2H, m), 7.79 (2H, dd, J=5.6, 8.8 Hz), 7.68-7.61 (2H, m), 7.33 (2H, t, J=8.8 Hz), 6.83 (1H, s), 3.41 (2H, s), 2.74 (2H, q, J=7.6 Hz), 1.30, (6H, s), 1.2 (3H, t, J=7.2 Hz).

LCMS (Method A): R_{T}=10.13 min. m/z=469 (ES+, M+H), 467 (ES−, M−H)

### Example 86

### 2-({[5-Chloro-3-(4-trifluoromethoxy-benzoyl)-thiophen-2-ylcarbamoyl]-methyl}-amino)-2-methyl-propionic acid

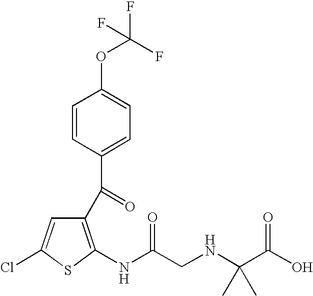

The required aminothiophene was prepared as described for Example 65. The acid bearing side chain was introduced by Methods A, D and E respectively, as described above.

¹H NMR (400 MHz, MeOD-d4) δ=7.88 (2H, d, J=8.4 Hz), 7.48 (2H, d, J=8.0 Hz), 7.09 (1H, s), 3.95 (2H, s), 1.51 (6H, s).

LCMS (Method A): R_{T}=10.18 min. m/z=466/464 (ES+, M+H), 464/462 (ES−, M−H)

### Example 87

### ({1-[3-(4-Chloro-benzoyl)-5-ethyl-thiophen-2-ylcarbamoyl]-ethyl}-methyl-amino)-acetic acid

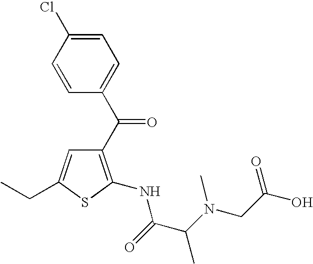

The required aminothiophene was prepared as described for Example 1. The acid bearing side chain was introduced by Methods A, D and E respectively, as described above.

¹H NMR (400 MHz, CDCl₃) δ=12.79 (1H, s), 7.67 (2H, d, J=8.5 Hz), 7.46 (2H, d, J=8.5 Hz), 6.75 (1H, s), 3.86-3.82 (1H, m), 3.57 (2H, d, J=4.8 Hz), 2.75 (2H, q, J=7.2 Hz), 2.57 (3H, s), 1.49 (3H, d, J=7.2 Hz), 1.28 (3H, t, J=7.2 Hz).

LCMS (Method A): R_{T}=9.78 min. m/z=409 (ES+, M+H), 407 (ES−, M−H)

### Example 88

### (3-{[5-Ethyl-3-(4-methoxy-benzoyl)-thiophen-2-ylcarbamoyl]-methylsulfanyl}-phenoxy)-acetic acid

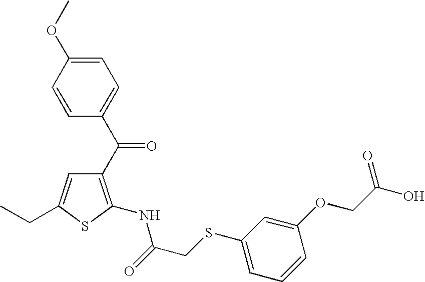

The required aminothiophene was prepared as described for Example 1 starting from (4-methoxybenzoyl)acetonitrile. The acid bearing side chain was introduced by Methods A, B, F and E respectively, as described above.

¹H NMR (400 MHz, CDCl₃) δ=7.74 (2H, d, J=8.8 Hz), 7.19 (1H, d, J=7.5 Hz), 7.01 (2H, d, J=7.5 Hz), 6.97 (2H, d, J=8.8 Hz), 6.79 (2H, d, J=7.5 Hz), 4.56 (2H, s), 3.88 (3H, s), 3.85 (2H, s), 2.72 (2H, q, J=7.6 Hz), 1.26 (3H, t, J=7.6 Hz).

LCMS (Method A): R_{T}=11.55 min. m/z=486 (ES+, M+H), 484 (ES−, M−H)

### Example 89

### (4-{[5-Ethyl-3-(4-methoxy-benzoyl)-thiophen-2-ylcarbamoyl]-methylsulfanyl}-phenoxy)-acetic acid

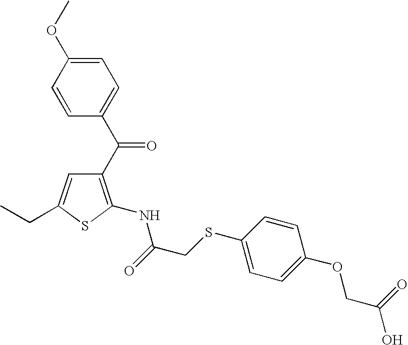

The required aminothiophene was prepared as described for Example 1 starting from (4-methoxybenzoyl)acetonitrile. The acid bearing side chain was introduced by Methods A, B, F and E respectively, as described above.

¹H NMR (400 MHz, CDCl₃) δ=7.76 (2H, d, J=8.8 Hz), 7.47 (2H, d, J=8.8 Hz), 6.98 (2H, d, J=8.8 Hz), 6.81 (2H, d, J=8.8 Hz), 4.55 (2H, s), 3.89 (3H, s), 3.76 (2H, s), 2.74 (2H, q, J=7.3 Hz), 1.27 (3H, t, J=7.3 Hz).

LCMS (Method A): R_{T}=11.52 min. m/z=486 (ES+, M+H), 484 (ES−, M−H)

### Example 90

### 3-{[5-Ethyl-3-(4-methoxy-benzoyl)-thiophen-2-ylcarbamoyl]-methylsulfanyl}-benzoic acid

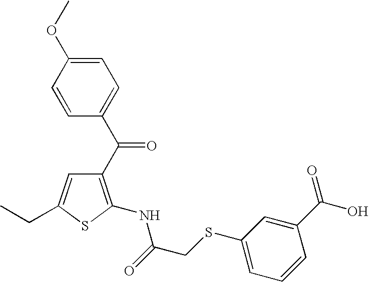

The required aminothiophene was prepared as described for Example 1 starting from (4-methoxybenzoyl)acetonitrile. The acid bearing side chain was introduced by Methods A and B respectively, as described above.

¹H NMR (400 MHz, CDCl₃) δ=12.81 (1H, bs), 8.21 (1H, s), 7.92 (1H, d, J=7.7 Hz), 7.75 (2H, d, J=8.9 Hz), 7.70 (1H, d, J=7.7 Hz), 7.39 (1H, d, J=7.7 Hz), 6.97 (2H, d, J=8.9 Hz), 6.81 (1H, s), 3.92 (2H, s), 3.87 (3H, s), 2.73 (2H, q, J=7.5 Hz), 1.27 (3H, t, J=7.5 Hz).

LCMS (Method A): R_{T}=11.55 min. m/z=456 (ES+, M+H), 454 (ES−, M−H)

### Example 91

### (4-{[5-Ethyl-3-(4-methoxy-benzoyl)-thiophen-2-ylcarbamoyl]-methylsulfanyl}-2-methyl-phenoxy)-acetic acid

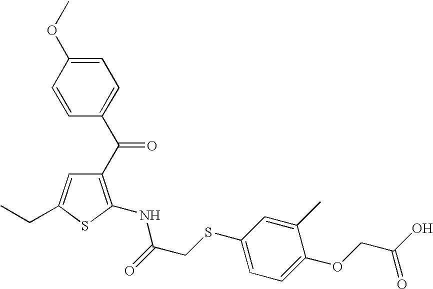

The required aminothiophene was prepared as described for Example 1 starting from (4-methoxybenzoyl)acetonitrile. The acid bearing side chain was introduced by Methods A, B, F and E respectively, as described above.

¹H NMR (400 MHz, CDCl₃) δ=12.69 (1H, s), 7.78 (2H, d, J=8.8 Hz), 7.35-7.31 (2H, m), 6.98 (2H, d, J=8.8 Hz), 6.81 (1H, s), 6.60 (1H, d, J=8.4 Hz), 4.59 (2H, s), 3.89 (3H, s), 3.76 (2H, s), 2.74 (2H, q, J=7.7 Hz), 2.10 (3H, s), 1.27 (3H, t, J=7.7 Hz).

LCMS (Method A): R_{T}=12.01 min. m/z=500 (ES+, M+H), 498 (ES−, M−H)

### Example 92

### 2-(4-{[5-Ethyl-3-(4-methoxy-benzoyl)-thiophen-2-ylcarbamoyl]-methylsulfanyl}-phenoxy)-2-methyl-propionic acid

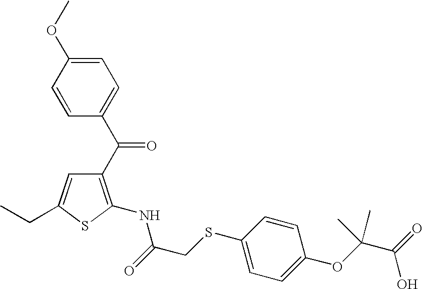

The required aminothiophene was prepared as described for Example 1. The acid bearing side chain was introduced by Methods A, B, F and E respectively, as described above.

¹H NMR (400 MHz, CDCl₃) δ=12.72 (1H, s), 7.75 (2H, d, J=8.8 Hz), 7.42 (2H, d, J=8.8 Hz), 6.97 (2H, d, J=8.8 Hz), 6.82 (3H, d, J=8.8 Hz), 3.89 (3H, s), 3.78 (2H, s), 2.74 (2H, q, J=7.3 HZ), 1.54 (6H, s), 1.27 (3H, t, J=7.3 Hz).

LCMS (Method A): R_{T}=12.19 min. m/z=514 (ES+, M+H), 512 (ES−, M−H)

### Example 93

### (4-{[5-trifluoromethoxy-benzoyl)-thiophen-2-ylcarbamoyl]-methylsulfanyl}-phenoxy)-acetic acid

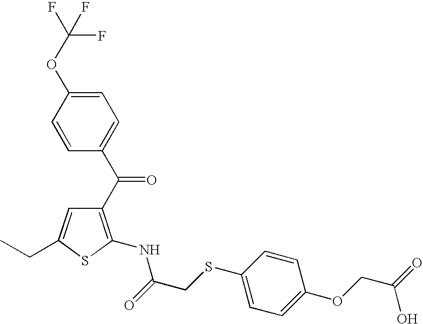

The required aminothiophene was prepared as described for Example 1. The acid bearing side chain was introduced by Methods A, B, F and E respectively, as described above.

¹H NMR (400 MHz, CDCl₃) δ=12.77 (1H, s), 7.79 (2H, d, J=8.8 Hz), 7.48 (2H, d, J=8.8 Hz), 7.33 (2H, d, J=8.8 Hz), 6.84 (2H, d, J=8.8 Hz), 6.74 (1H, s), 4.61 (2H, s), 3.79 (2H, s), 2.74 (2H, q, J=7.7 Hz), 1.27 (3H, t, J=7.7 Hz).

LCMS (Method A): R_{T}=12.05 min. m/z=540 (ES+, M+H), 538 (ES−, M−H)

### Example 94

### (4-{[5-Ethyl-3-(4-trifluoromethoxy-benzoyl)-thiophen-2-ylcarbamoyl]-methylsulfanyl}-2-methyl-phenoxy)-acetic acid

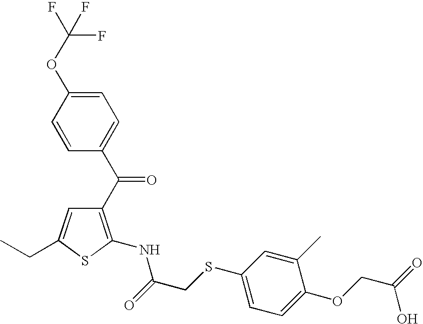

The required aminothiophene was prepared as described for Example 1. The acid bearing side chain was introduced by Methods A, B, F and E respectively, as described above.

¹H NMR (400 MHz, CDCl₃) δ=12.74 (1H, s), 7.79 (2H, d, J=8.8 Hz), 7.34-7.31 (4H, m), 6.74 (1H, s), 6.63 (1H, d, J=8.4 Hz), 4.62 (2H, s), 3.78 (2H, s), 2.74 (2H, q, J=7.7 Hz), 2.19 (3H, s), 1.27 (3H, t, J=7.7 Hz).

LCMS (Method A): R_{T}=12.44 min. m/z=554 (ES+, M+H), 552 (ES−, M−H)

### Example 95

### 2-(4-{[5-Ethyl-3-(4-trifluoromethoxy-benzoyl)-thiophen-2-ylcarbamoyl]-methylsulfanyl}-phenoxy)-2-methyl-propionic acid

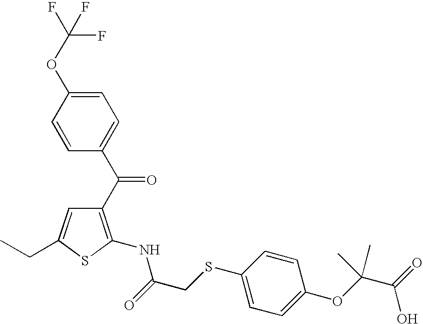

The required aminothiophene was prepared as described for Example 1. The acid bearing side chain was introduced by Methods A, B, F and E respectively, as described above.

¹H NMR (400 MHz, CDCl₃) δ=12.74 (1H, S), 7.78 (2H, d, J=8.5 Hz), 7.40 (2H, d, J=8.1 Hz), 7.32 (2H, d, J=8.1 Hz), 6.83 (2H, d, J=8.5 Hz), 6.78 (1H, s), 3.80 (2H, s), 2.74 (2H, q, J=7.5 Hz), 1.56 (6H, s), 1.27 (3H, t, J=7.5 Hz).

LCMS (Method A): R_{T}=13.12 min. m/z=568 (ES+, M+H), 566 (ES−, M−H)

### Example 96

### 3-(4-{[5-Ethyl-3-(4-trifluoromethoxy-benzoyl)-thiophen-2-ylcarbamoyl]-methylsulfanyl}-phenyl)-propionic acid

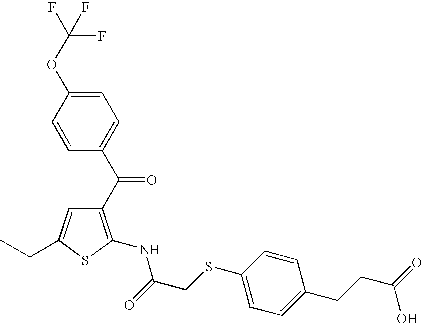

The required aminothiophene was prepared as described for Example 1. The acid bearing side chain was introduced by Methods A, B and C respectively, as described above.

¹H NMR (400 MHz, CDCl₃) δ=12.79 (1H, s), 7.78 (2H, d, J=8.8 Hz), 7.39 (2H, d, J=8.4 Hz), 7.32 (2H, d, J=8.8 Hz), 7.13 (2H, d, J=8.4 Hz), 6.74 (1H, s), 3.84 (2H, s), 2.88 (2H, t, J=7.7 Hz), 2.74 (2H, q, J=7.7 Hz), 2.62 (2H, t, J=7.7 Hz), 1.27 (3H, t, J=7.7 Hz).

LCMS (Method A): R_{T}=12.78 min. m/z=538 (ES+, M+H), 536 (ES−, M−H)

### Example 97

### {4-[3-(4-Chloro-benzoyl)-5-ethyl-thiophen-2-ylcarbamoyl]-phenoxy}-acetic acid

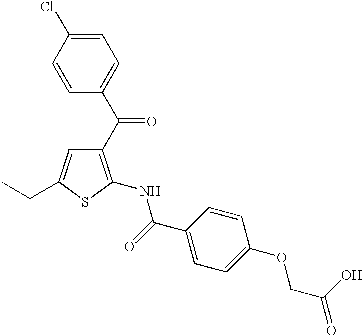

The required aminothiophene was prepared as described for Example 1. The required benzoyl chloride, (4-chlorocarbonyl-phenoxy)-acetic acid tert-butyl ester, was prepared from 4-hydroxybenzaldehyde by Methods F, G and H, reacted with the aminothiophene by Method A, and the tert-butyl ester cleaved by Method E.

¹H NMR (400 MHz, CDCl₃) δ=12.98 (1H, s), 8.04 (2H, d, J=8.8 Hz), 7.68 (2H, d, J=8.4 Hz), 7.47 (2H, d, J=8.4 Hz), 7.03 (2H, d, J=8.8 Hz), 6.76 (1H, s), 4.69 (2H, s), 3.41 (1H, s), 2.76 (2H, q, J=7.5 Hz), 1.29 (3H, t, J=7.5 Hz).

LCMS (Method A): R_{T}=13.25 min. m/z=444 (ES+, M+H), 442 (ES−, M−H)

### Example 98

### {3-[5-Ethyl-3-(4-trifluoromethoxy-benzoyl)-thiophen-2-ylcarbamoyl]-phenylsulfanyl}-acetic acid

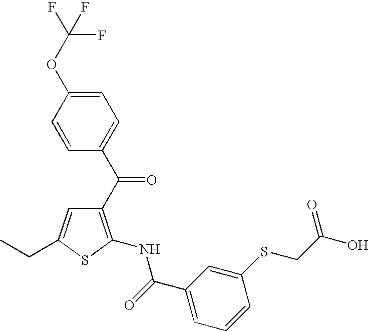

The required aminothiophene was prepared as described for Example 1. The required benzoyl chloride, (3-Chlorocarbonyl-phenylsulfanyl)-acetic acid tert-butyl ester, was prepared from 3-mercaptobenzaldehyde by Methods F, G and H, reacted with the aminothiophene by Method A, and the tert-butyl ester cleaved by Method E.

¹H NMR (400 MHz, CDCl₃) δ=12.92 (1H, s), 8.12 (1H, s), 7.95 (1H, d, J=7.7 Hz), 7.81 (2H, d, J=8.4 Hz), 7.68 (1H, d, J=7.7 Hz), 7.50 (1H, d, J=7.7 Hz), 7.35 (2H, d, J=8.4 Hz), 6.81 (1H, s), 3.71 (2H, s), 2.79 (2H, q, J=7.3 Hz), 1.32 (3H, t, J=7.3 Hz).

LCMS (Method A): R_{T}=13.37 min. m/z=510 (ES+, M+H), 508 (ES−, M−H)

### Example 99

### {4-[5-Ethyl-3-(4-trifluoromethoxy-benzoyl)-thiophen-2-ylcarbamoyl]-phenoxy}-acetic acid

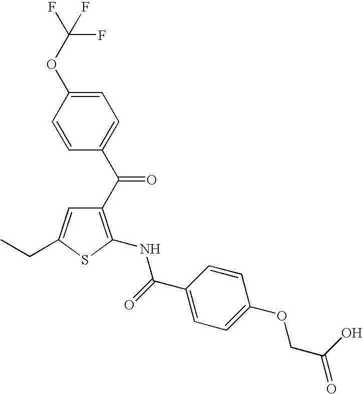

The required aminothiophene was prepared as described for Example 1. The required benzoyl chloride, (4-chlorocarbonyl-phenoxy)-acetic acid tert-butyl ester, was prepared from 4-hydroxybenzaldehyde by Methods F, G and H, reacted with the aminothiophene by Method A, and the tert-butyl ester cleaved by Method E.

¹H NMR (400 MHz, CDCl₃) δ=13.01 (1H, s), 8.07 (2H, d, J=9.2 Hz), 7.81 (2H, d, J=8.8 Hz), 7.35 (2H, d, J=9.2 Hz), 7.06 (2H, d, J=8.8 Hz), 6.79 (1H, s), 4.78 (2H, s), 2.78 (2H, q, J=7.5 Hz), 1.31 (3H, t, J=7.5 Hz).

LCMS (Method A): R_{T}=13.27 min. m/z=494 (ES+, M+H), 492 (ES−, M−H)

### Example 100

### {3-[5-Ethyl-3-(4-trifluoromethoxy-benzoyl)-thiophen-2-ylcarbamoyl]-phenoxy}-acetic acid

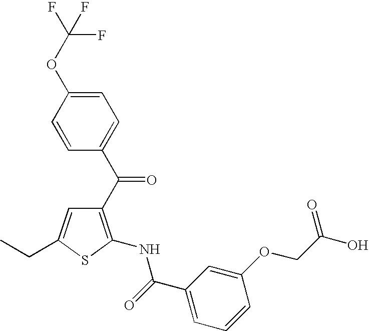

The required aminothiophene was prepared as described for Example 1. The required benzoyl chloride, (3-chlorocarbonyl-phenoxy)-acetic acid tert-butyl ester, was prepared from 3-hydroxybenzaldehyde by Methods F, G and H, reacted with the aminothiophene by Method A, and the tert-butyl ester cleaved by Method E.

¹H NMR (400 MHz, CDCl₃) δ=7.79 (2H, d, J=8.8 Hz), 7.64 (2H, d, J=7.3 Hz), 7.43 (1H, dd, J=16.1, 8.4 Hz), 7.32 (2H, d, J=7.7 Hz), 7.18 (1H, d, J=8.8 Hz), 6.78 (1H, s, J=8.4 Hz), 4.67 (2H, s), 2.76 (2H, qd, J=7.7, 1.1 Hz), 1.29 (3H, t, J=7.7 Hz).

LCMS (Method A): R_{T}=13.20 min. m/z=494 (ES+, M+H), 492 (ES−, M−H)

### Example 101

### {4-[5-Ethyl-3-(4-trifluoromethoxy-benzoyl)-thiophen-2-ylcarbamoyl]-2-methyl-phenoxy}-acetic acid

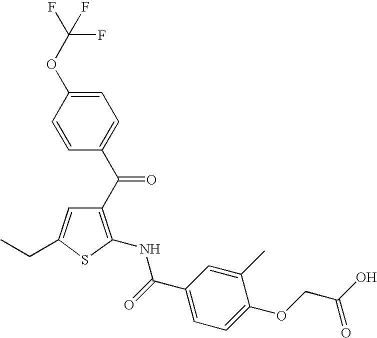

The required aminothiophene was prepared as described for Example 1. The required benzoyl chloride, (4-chlorocarbonyl-2-methyl-phenoxy)-acetic acid tert-butyl ester, was prepared from 4-hydroxy-2-methylbenzaldehyde by Methods F, G and H, reacted with the aminothiophene by Method A, and the tert-butyl ester cleaved by Method E.

¹H NMR (400 MHz, CDCl₃) δ=7.91 (2H, d, J=8.1 Hz), 7.80 (2H, d, J=8.8 Hz), 7.35 (2H, d, J=8.8 Hz), 6.84 (1H, d, J=8.1 Hz), 6.78 (1H, s), 4.78 (2H, s), 2.78 (2H, qd, J=7.7, 1.1 Hz), 2.39 (3H, s), 1.31 (3H, t, J=7.7 Hz).

LCMS (Method A): R_{T}=13.74 min. m/z=508 (ES+, M+H), 506 (ES−, M−H)

### Example 102

### 2-{4-[5-Ethyl-3-(4-trifluoromethoxy-benzoyl)-thiophen-2-ylcarbamoyl]-phenoxy}-2-methyl-propionic acid

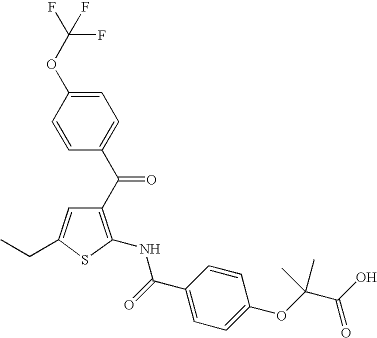

The required aminothiophene was prepared as described for Example 1. The required benzoyl chloride, 2-(4-chlorocarbonyl-phenoxy)-2-methyl-propionic acid tert-butyl ester, was prepared from 4-hydroxybenzaldehyde by Methods F, G and H, reacted with the aminothiophene by Method A, and the tert-butyl ester cleaved by Method E.

¹H NMR (400 MHz, CDCl₃) δ=8.02 (2H, d, J=8.8 Hz), 7.80 (2H, d, J=8.8 Hz), 7.34 (2H, d, J=8.8), 7.01 (2H, d, J=8.8 Hz), 6.79 (1H, s), 2.78 (2H, qd, J=7.3, 1.1 Hz), 1.69 (6H, s), 1.31 (3H, t, J=7.3 Hz).

LCMS (Method A): R_{T}=13.79 min. m/z=522 (ES+, M+H), 520 (ES−, M−H)

### Example 103

### {4-[5-Ethyl-3-(4-trifluoromethoxy-benzoyl)-thiophen-2-ylcarbamoyl]-phenylsulfanyl}-acetic acid

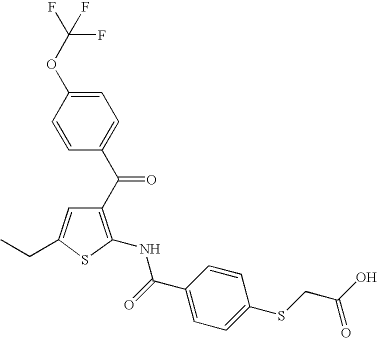

The required aminothiophene was prepared as described for Example 1. The required benzoyl chloride, (4-Chlorocarbonyl-phenylsulfanyl)-acetic acid tert-butyl ester, was prepared from 4-mercaptobenzoic acid by Methods F, G and H, reacted with the aminothiophene by Method A, and the tert-butyl ester cleaved by Method E.

¹H NMR (400 MHz, CDCl₃) δ=13.06 (1H, s), 8.01 (2H, d, J=8.8 Hz), 7.80 (2H, d, J=8.8 Hz), 7.48 (2H, d, J=8.4 Hz), 7.35 (2H, d, J=8.4 Hz), 6.80 (1H, s), 3.80 (2H, s), 2.78 (2H, q, J=7.7 Hz), 1.31 (3H, t, J=7.7 Hz).

LCMS (Method A): R_{T}=13.02 min. m/z=510 (ES+, M+H), 508 (ES−, M−H)

### Example 104

### 4-[5-Ethyl-3-(4-trifluoromethoxy-benzoyl)-thiophen-2-ylcarbamoyl]-butyric acid

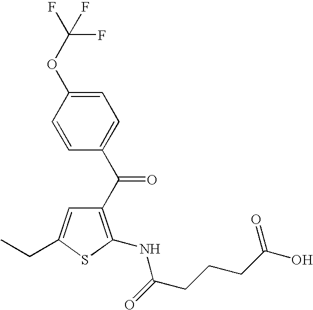

The title compound was made by an analogous procedure to Example 1, using glutaric anhydride in the final step.

¹H NMR (400 MHz, CDCl₃) δ=11.97 (1H, s), 7.76 (2H, d, J=8.8 Hz), 7.32 (2H, d, J=8.8 Hz), 6.72 (1H, s), 2.74 (2H, dq, J=1.1, 7.7 Hz), 2.65 (2H, dd, J=7.3 Hz), 2.52 (2H, dd, J=7.3 Hz), 2.12 (2H, app quint, J=7.3 Hz), 1.28 (3H, t, J=7.7 Hz).

LCMS (Method A): R_{T}=12.06 min. m/z=430 (ES+, M+H), 428 (ES−, M−H)

### Example 105

### {3-[5-Ethyl-3-(4-trifluoromethoxy-benzoyl)-thiophen-2-ylcarbamoyl]-propylsulfanyl}-acetic acid

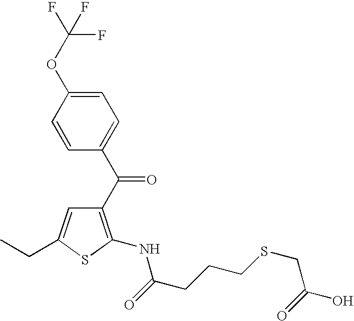

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=11.93 (1H, s), 7.76 (2H, d, J=8.4 Hz), 7.32 (2H, d, J=8.4 Hz), 6.72 (1H, s), 3.28 (2H, s), 2.81-2.68 (6H, m), 2.14-2.07 (2H, m), 1.28 (3H, t, J=7.5 Hz).

LCMS (Method A): R_{T}=12.30 min. m/z=476 (ES+, M+H), 474 (ES−, M−H)

### Example 106

### {1-[5-Ethyl-3-(4-trifluoromethoxy-benzoyl)-thiophen-2-ylcarbamoyl]-ethylsulfanyl}-acetic acid

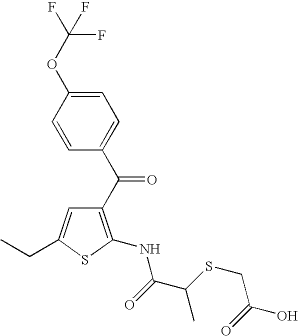

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.52 (1H, s), 7.78 (2H, d, J=8.8 Hz), 7.32 (2H, d, J=8.8 Hz), 6.76 (1H, s), 3.90 (1H, q, J=7.3 Hz), 3.45 (1H, d, J=15.4 Hz), 3.36 (1H, d, J=15.4 Hz), 2.75 (2H, dq, J=7.3, 1.1 Hz), 1.64 (3H, d, J=7.3 Hz), 1.28 (3H, t, J=7.3 Hz).

LCMS (Method A): R_{T}=12.25 min. m/z=462 (ES+, M+H), 460 (ES−, M−H)

### Example 107

### {1-[5-Ethyl-3-(4-trifluoromethoxy-benzoyl)-thiophen-2-ylcarbamoyl]-1-methyl-ethylsulfanyl}-acetic acid

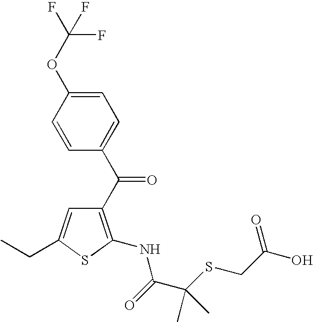

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.78 (1H, s), 7.79 (2H, d, J=8.8 Hz), 7.32 (2H, d, J=8.8 Hz), 6.76 (1H, s), 3.42 (2H, s), 2.75 (2H, dq, J=1.1, 7.3 Hz), 1.70 (6H, s), 1.28 (3H, t, J=7.3 Hz).

LCMS (Method A): R_{T}=12.71 min. m/z=476 (ES+, M+H), 474 (ES−, M−H)

### Example 108

### 2-{1-[5-Ethyl-3-(4-trifluoromethoxy-benzoyl)-thiophen-2-ylcarbamoyl]-1-methyl-ethylsulfanyl}-propionic acid

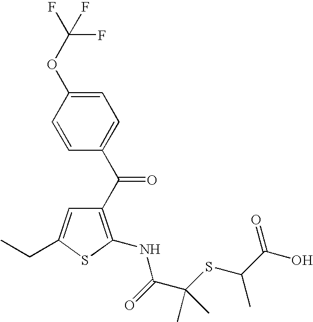

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.77 (1H, s), 7.79 (2H, d, J=8.8 Hz), 7.33 (2H, d, J=8.8 Hz), 6.76 (1H, s), 3.53 (2H, q, J=7.3. Hz), 2.75 (2H, q, J=7.1 Hz), 1.71 (3H, s), 1.70 (3H, s), 1.45 (3H, s), 1.28 (3H, t, J=7.1 Hz).

LCMS (Method A): R_{T}=13.04 min. m/z=490 (ES+, M+H), 488 (ES−, M−H)

### Example 109

### {1-[5-Ethyl-3-(4-trifluoromethoxy-benzoyl)-thiophen-2-ylcarbamoyl]-propylsulfanyl}-acetic acid

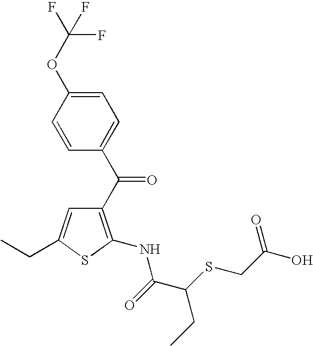

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.33 (1H, s), 7.76 (2H, d, J=8.2 Hz), 7.27 (2H, d, J=8.2 Hz), 6.70 (1H, s), 3.67 (1H, broad s), 3.28 (2H, broad s), 2.71 (2H, q, J=7.3 Hz), 2.04-1.97 (1H, m), 1.86-1.79 (1H, m), 1.25 (3H, t, J=7.3 Hz), 1.00 (3H, t, J=6.9 Hz).

LCMS (Method A): R_{T}=12.90 min. m/z=476 (ES+, M+H), 474 (ES−, M−H)

### Example 110

### 2-{1-[5-Ethyl-3-(4-trifluoromethoxy-benzoyl)-thiophen-2-ylcarbamoyl]-propylsulfanyl}-propionic acid

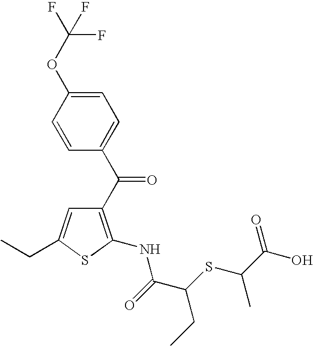

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=7.85 (2H, d, J=8.8 Hz), 7.52 (2H, d, J=8.8 Hz), 6.82 (1H, s), 3.87-3.82 (1H, m), 3.41-3.36 (1H, m), 2.73 (2H, q, J=7.3 Hz), 1.91-1.83 (1H, m), 1.79-1.67 (1H, m), 1.28 (3H, d, J=6.9 Hz), 1.21 (3H, t, J=7.6 Hz), 0.93 (3H, t, J=7.3 Hz).

LCMS (Method A): R_{T}=12.90 min. m/z=476 (ES+, M+H), 474 (ES−, M−H)

### Example 111

### 2-{[5-Ethyl-3-(4-methoxy-benzoyl)-thiophen-2-ylcarbamoyl]-methylsulfanyl}-propionic acid

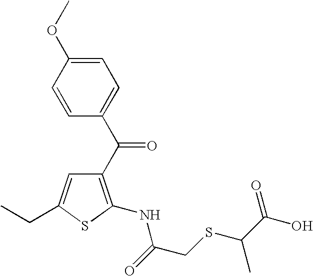

The required aminothiophene was prepared as described for Example 1 starting from (4-methoxybenzoyl)acetonitrile. The acid bearing side chain was introduced by Methods A, B and C respectively as described above.

¹H NMR (400 MHz, CDCl₃) δ=12.57 (1H, s), 7.75 (2H, d, J=8.8 Hz), 6.98 (2H, d, J=8.8 Hz), 6.83 (1H, s), 3.89 (3H, s), 3.81-3.54 (3H, m), 2.76 (2H, q, J=7.7 Hz), 1.52 (3H, d, J=7.3 Hz), 1.29 (3H, t, J=7.7 Hz).

LCMS (Method A): R_{T}=11.28 min. m/z=408 (ES+, M+H), 406 (ES−, M−H)

### Example 112

### 2-{[5-Ethyl-3-(4-trifluoromethoxy-benzoyl)-thiophen-2-ylcarbamoyl]-methylsulfanyl}-propionic acid

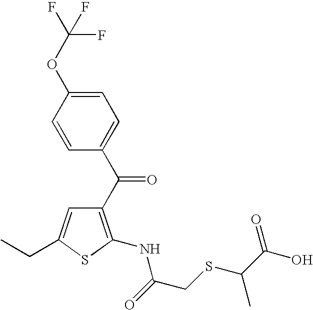

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.59 (1H, s), 7.78 (2H, d, J=8.2 Hz), 7.32 (2H, d, J=8.2 Hz), 6.75 (1H, s), 3.82-3.54 (3H, m), 2.75 (2H, dq, J=1.1, 7.3 Hz), 1.51 (3H, d, J=7.3 Hz), 1.28 (3H, t, J=7.3 Hz).

LCMS (Method A): R_{T}=12.42 min. m/z=462 (ES+, M+H), 460 (ES−, M−H)

### Example 113

### {[5-Ethyl-3-(4-trifluoromethoxy-benzoyl)-thiophen-2-ylcarbamoyl]-phenyl-methylsulfanyl}-acetic acid

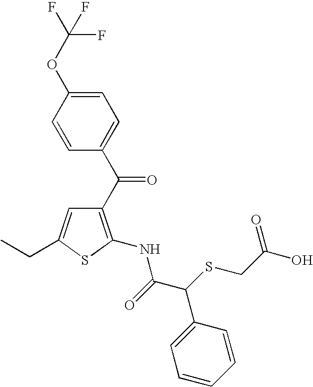

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.36 (1H, s), 7.70 (2H, d, J=8.8 Hz), 7.46 (2H, d, J=7.7 Hz), 7.32-7.27 (3H, m), 7.23 (2H, d, J=8.8 Hz), 6.65 (1H, s), 5.11 (1H, s), 3.27 (2H, pair of d, J=15 Hz), 2.67 (2H, q, J=7.3 Hz), 1.21 (3H, t, J=7.3 Hz).

LCMS (Method A): R_{T}=13.17 min. i/z=524 (ES+, M+H), 522 (ES−, M−H)

### Example 114

### {1-[5-Ethyl-3-(4-trifluoromethoxy-benzoyl)-thiophen-2-ylcarbamoyl]-2-methyl-propylsulfanyl}-acetic acid

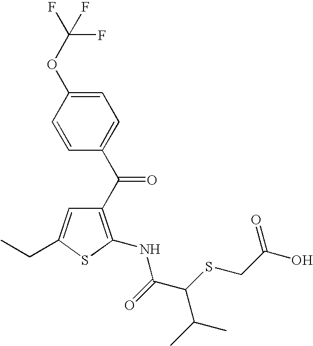

The required aminothiophene was prepared as described for Example 1 starting from (4-trifluoromethoxybenzoyl)acetonitrile. The acid bearing side chain was introduced by Methods A, B and C respectively as described above.

¹H NMR (400 MHz, CDCl₃) δ=12.55 (1H, s), 7.79 (2H, d, J=8.8 Hz), 7.32 (2H, d, J=8.8 Hz), 6.76 (1H, s), 3.57 (1H, d, J=7.7 Hz), 3.35 (2H, s), 2.75 (2H, dq, J=1.1, 7.7 Hz), 2.37-2.30 (1H, m), 1.29 (3H, t, J=7.7 Hz), 1.12 (3H, s), 1.10 (3H, s).

LCMS (Method A): R_{T}=13.36 min. m/z=490 (ES+, M+H), 488 (ES−, M−H)

### Example 115

### {4-[5-Ethyl-3-(4-trifluoromethoxy-benzoyl)-thiophen-2-ylcarbamoyl]-phenylamino}-acetic acid

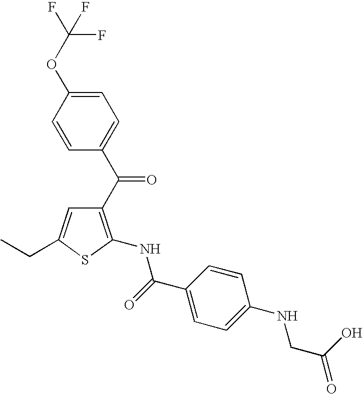

The required aminothiophene was prepared as described for Example 1 starting from (4-trifluoromethoxybenzoyl)acetonitrile. The required benzoyl chloride, (4-chlorocarbonyl-phenyl)-carbamic acid tert-butyl ester, was prepared from 4-tert-butoxycarbonylamino-benzoic acid by Method H, reacted with the aminothiophene by Methods A, D, and the tert-butyl ester cleaved by Method E.

¹H NMR (400 MHz, CDCl₃) δ=12.94 (1H, s), 7.97 (2H, d, J=8.4 Hz), 7.80 (2H, d, J=8.8 Hz), 7.34 (2H, d, J=8.4 Hz), 6.77 (1H, s), 6.70 (2H, d, J=8.8 Hz), 4.07 (2H, s), 2.77 (2H, dq, J=7.3, 1.1 Hz), 1.30 (3H, t, J=7.3 Hz).

LCMS (Method A): R_{T}=12.90 min. m/z=493 (ES+, M+H), 491 (ES−, M−H)

### Example 116

### 3-{4-[5-Ethyl-3-(4-trifluoromethoxy-benzoyl)-thiophen-2-ylcarbamoyl]-phenyl}-propionic acid

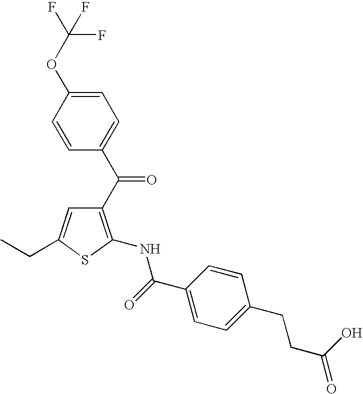

The required aminothiophene was prepared as described for Example 1 starting from (4-trifluoromethoxybenzoyl)acetonitrile and reacted with 4-bromobenzoyl chloride using Method A. The acid bearing side chain was introduced by Methods I, using methyl acrylate, J and C respectively as described above.

¹H NMR (400 MHz, CDCl₃) δ=13.02 (1H, s), 8.02 (2H, d, J=8.4 Hz), 7.81 (2H, d, J=8.8 Hz), 7.39 (2H, d, J=8.4 Hz), 7.35 (2H, d, J=8.8 Hz), 6.80 (1H, s), 3.06 (2H, app dd, J=7.3 Hz), 2.81-2.72 (4H, m), 1.31 (3H, t, J=7.7 Hz).

LCMS (Method A): R_{T}=13.31 min. m/z=492 (ES+, M+H), 490 (ES−, M−H)

### Example 117

### (4-{[5-Ethyl-3-(4-trifluoromethoxy-benzoyl)-thiophen-2-ylcarbamoyl]-methoxy}-phenoxy)-acetic acid

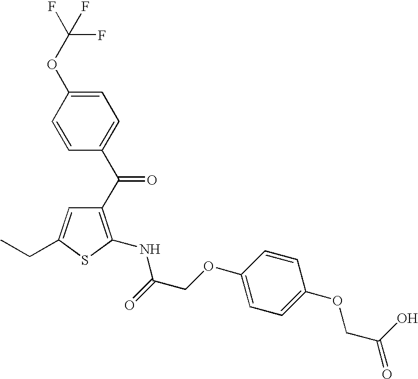

The required aminothiophene was prepared as described for Example 1 starting from (4-trifluoromethoxybenzoyl)acetonitrile and the chloride formed by reacting the aminothiophene with chloroacetyl chloride using Method A. The required phenol was prepared from hydroquinone and tert-butyl bromoacetate using Method K and then reacted with the chloride also using Method K. The tert-butyl ester was cleaved using Method E.

¹H NMR (400 MHz, CDCl₃) δ=12.47 (1H, s), 7.86 (2H, d, J=9.1 Hz), 7.54 (2H, d, J=9.1 Hz), 7.04 (2H, d, J=9.1 Hz), 6.90 (2H, d, J=9.1 Hz), 6.86 (1H, s), 4.86 (2H, s), 4.56 (2H, s), 2.75 (2H, dq, J=7.3, 1.1 Hz), 1.21 (3H, t, J=7.3 Hz).

LCMS (Method A): R_{T}=12.76 min. m/z=524 (ES+, M+H), 522 (ES−, M−H)

### Example 118

### {1-[5-Ethyl-3-(4-trifluoromethoxy-benzoyl)-thiophen-2-ylcarbamoyl]-1-methyl-ethylamino}-acetic acid

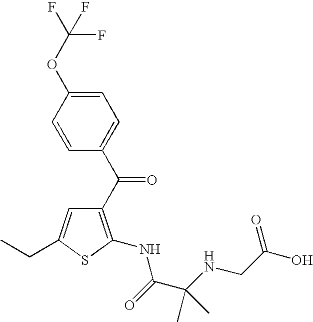

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, CDCl₃) δ=7.74 (2H, d, J=8.4 Hz), 7.28 (2H, d, J=8.4 Hz), 6.70 (1H, s), 3.45 (2H, s), 2.71 (2H, q, J=7.3 Hz), 1.58 (6H, s), 1.24 (3H, t, J=7.3 Hz).

LCMS (Method A): R_{T}=9.59 min. m/z=459 (ES+, M+H), 457 (ES−, M−H)

### Example 119

### (R)-1-{1-[5-Ethyl-3-(4-trifluoromethoxy-benzoyl)-thiophen-2-ylcarbamoyl]-1-methyl-ethyl}-pyrrolidine-2-carboxylic acid

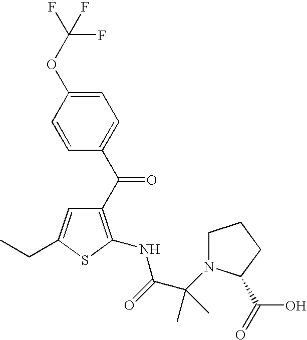

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, CDCl₃) δ=12.86 (1H, s), 7.79 (2H, d, J=8.4 Hz), 7.35 (2H, d, J=8.4 Hz), 6.84 (1H, s), 4.88 (1H, broad s), 4.25 (1H, broad s), 3.64 (1H, broad s), 2.81-2.10 (4H, broad multiple signals), 2.80 (2H, q, J=7.7 Hz), 2.04 (3H, s), 2.01 (3H, s), 1.31 (3H, t, J=7.7 Hz).

LCMS (Method A): R_{T}=10.42 min. m/z=499 (ES+, M+H), 497 (ES−, M−H)

### Example 120

### (R)-1-{1-[5-Ethyl-3-(4-trifluoromethoxy-benzoyl)-thiophen-2-ylcarbamoyl]-propyl}-pyrrolidine-2-carboxylic acid

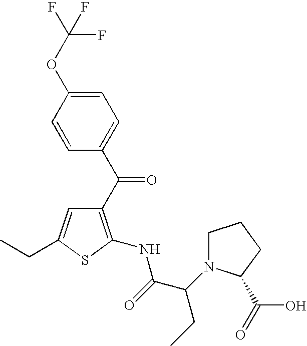

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, CDCl₃) Diastereosiomer 1: δ=12.40 (1H, s), 7.78 (2H, d, J=8.8 Hz), 7.33 (2H, d, J=8.8 Hz), 6.78 (1H, s), 4.05 (1H, dd, J=8.4 Hz), 3.59 (1H, dd, J=7.3 Hz), 3.31-3.23 (1H, m), 3.08-3.02 (1H, m), 2.78-2.73 (2H, m), 2.34-1.82 (6H, m), 1.30 (3H, t, J=7.7 Hz), 1.09 (3H, t, J=7.3 Hz).

Diastereoisomer 2: δ=12.38 (1H, s), 7.77 (2H, d, J=8.8 Hz), 7.33 (2H, d, J=8.8 Hz), 6.76 (1H, s), 3.78 (1H, dd, J=8.4 Hz), 3.53 (1H, dd, J=7.3 Hz), 3.31-3.23 (1H, m), 2.83-2.79 (1H, m), 2.78-2.73 (2H, m), 2.34-1.82 (6H, m), 1.29 (3H, t, J=7.7 Hz), 1.07 (3H, t, J=7.3 Hz).

LCMS (Method A): R_{T}=10.51 and 11.14 min. m/z=499 (ES+, M+H), 497 (ES−, M−H)

### Example 121

### {1-[5-Ethyl-3-(4-trifluoromethoxy-benzoyl)-thiophen-2-ylcarbamoyl]-propylamino}-acetic acid

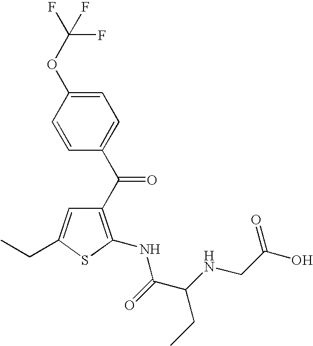

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, CDCl₃) δ=12.11 (1H, s), 7.75 (2H, d, J=7.7 Hz), 7.27 (2H, d, J=7.7 Hz), 6.70 (1H, s), 4.69 (1H, broad s), 4.30 (1H, broad s), 4.11 (1H, broad s), 2.71 (2H, broad q, J=7.3 Hz), 2.22 (2H, broad d), 1.24 (3H, t, J=7.3 Hz), 1.03 (3H, broad s).

LCMS (Method A): R_{T}=10.05 min. m/z=459 (ES+, M+H), 457 (ES−, M−H)

### Example 122

### ({[5-Ethyl-3-(4-trifluoromethoxy-benzoyl)-thiophen-2-ylcarbamoyl]-phenyl-methyl}-amino)-acetic acid

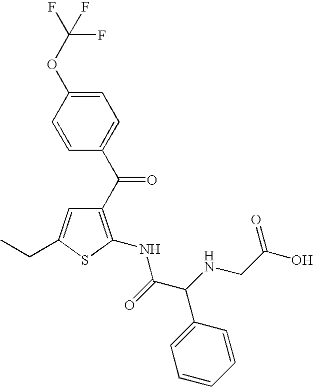

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, CDCl₃) δ=11.92 (1H, s), 7.82 (2H, broad d, J=5.5 Hz), 7.58 (2H, d, J=8.2 Hz), 7.43 (2H, broad d, J=5.5 Hz), 7.21 (2H, d, J=8.2 Hz), 6.59 (1H, s), 5.94 (1H, s), 4.13 (1H, d, J=16.5 Hz), 3.91 (1H, d, J=16.5 Hz), 2.63 (2H, q, J=7.3 Hz), 1.17 (3H, t, J=7.3 Hz).

LCMS (Method A): R_{T}=11.94 min. m/z=507 (ES+, M+H), 505 (ES−, M−H)

### Example 123

### (R)-1-{1-[5-Ethyl-3-(4-trifluoromethoxy-benzoyl)-thiophen-2-ylcarbamoyl]-ethyl}-pyrrolidine-2-carboxylic acid

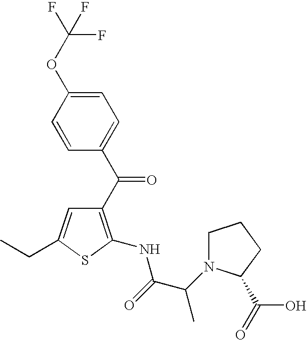

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, d₃-MeOD) δ=7.89 (2H, d, J=8.4 Hz), 7.47 (2H, d, J=8.4 Hz), 6.89 (1H, s), 4.70-4.65 (1H, m), 4.52-4.49 (1H, m), 3.81-3.67 (2H, m), 2.82 (2H, q, J=7.3 Hz), 2.59-2.51 (1H, m), 2.32-2.08 (3H, m), 1.74 (3H, dd, J=7.3 Hz), 1.31 (3H, t, J=7.3 Hz).

LCMS (Method A): R_{T}=9.94 and 10.56 min (diastereoisomers). m/z=485 (ES+, M+H), 483 (ES−, M−H)

### Example 124

### (R)-1-{[5-Ethyl-3-(4-trifluoromethoxy-benzoyl)-thiophen-2-ylcarbamoyl]-phenyl-methyl}-pyrrolidine-2-carboxylic acid

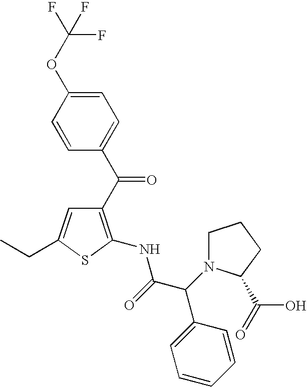

The required aminothiophene was prepared as described for Example 1 starting from (4-trifluoromethoxybenzoyl)acetonitrile. The acid bearing side chain was introduced by Methods A, D and E respectively as described above.

¹H NMR (400 MHz, d₃-MeOD) Diastereosiomer 1: δ=7.68 (2H, d, J=8.8 Hz), 7.56-7.44 (5H, m), 7.30 (2H, d, J=8.8 Hz), 6.72 (1H, s), 5.71 (1H, s), 4.40 (1H, t, J=6.2 Hz), 4.20-4.17 (2H, m), 2.69, (2H, q, J=7.3 Hz), 2.44-2.39 (1H, m), 2.17-2.00 (3H, m), 1.19 (3H, t, J=7.3 Hz).

Diastereoisomer 2: δ=7.66 (2H, d, J=8.8 Hz), 7.56-7.44 (5H, m), 7.30 (2H, J=8.8 Hz), 6.71 (1H, s), 5.70 (1H, s), 3.84-3.80 (1H, m), 3.50-3.46 (2H, m), 2.68 (2H, q, J=7.3 Hz), 2.36-2.31 (1H, m), 2.17-2.00 (3H, m), 1.18 (3H, t, J=7.3 Hz).

LCMS (Method A): R_{T}=12.28 and 13.28 min. m/z=547 (ES+, M+H), 545 (ES−, M−H)

### Example 125

### (S)-2-(Ethyl-{[5-ethyl-3-(4-trifluoromethoxy-benzoyl)-thiophen-2-ylcarbamoyl]-methyl}-amino)-propionic acid

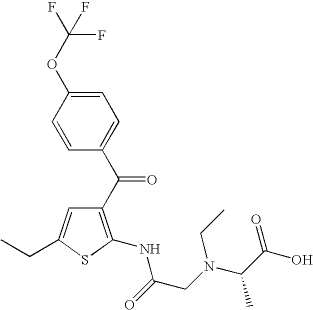

The required aminothiophene was prepared as described for Example 1 starting from (4-trifluoromethoxybenzoyl)acetonitrile. The acid bearing side chain was introduced by Methods A, D and K respectively as described above. The tert-butyl group was cleaved using Method E.

¹H NMR (400 MHz, d₃-MeOD) δ=7.77 (2H, d, J=8.8 Hz), 7.35 (2H, d, J=8.8 Hz), 6.76 (1H, s), 4.52-4.30 (3H, m), 3.35-3.26 (2H, m), 2.70 (2H, dq, J=1.1, 7.3 Hz), 1.56 (3H, d, J=7.3 Hz), 1.27 (3H, t, J=7.3 Hz), 1.20 (3H, t, J=7.3 Hz).

LCMS (Method A): R_{T}=12.59 min. m/z=473 (ES+, M+H), 471 (ES−, M−H)

### Example 126

### (S)-2-({[5-Ethyl-3-(4-trifluoromethoxy-benzoyl)-thiophen-2-ylcarbamoyl]-methyl}-methanesulfonyl-amino)-propionic acid

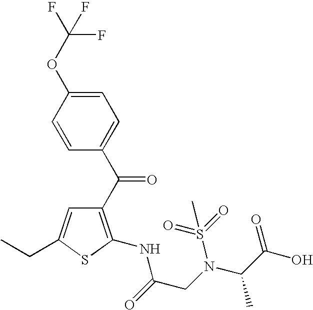

The required aminothiophene was prepared as described for Example 1 starting from (4-trifluoromethoxybenzoyl)acetonitrile. The acid bearing side chain was introduced by Methods A, D and K respectively as described above. The tert-butyl group was cleaved using Method E.

¹H NMR (400 MHz, d₃-MeOD) δ=7.75 (2H, d, J=8.8 Hz), 7.33 (2H, d, J=8.8 Hz), 6.71 (1H, s), 4.71 (1H, q, J=7.5 Hz), 4.30 (1H, d, J=18.5 Hz), 4.07 (1H, d, J=18.5 Hz), 3.01 (3H, s), 2.67 (2H, dq, J=1.1, 7.5 Hz), 1.50 (3H, d, J=7.5 Hz), 1.18 (3H, t, J=7.5 Hz).

LCMS (Method A): R_{T}=12.33 min. m/z=523 (ES+, M+H), 521 (ES−, M−H)

### Example 127

### 2-{[5-Ethyl-3-(4-trifluoromethoxy-benzoyl)-thiophen-2-ylcarbamoyl]-methylsulfanyl}-butyric acid

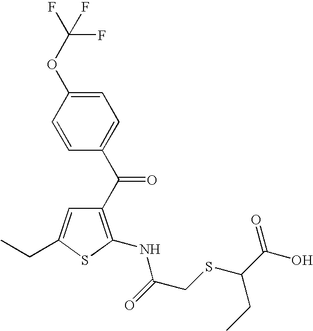

The required aminothiophene was prepared as described for Example 1 starting from (4-trifluoromethoxybenzoyl)acetonitrile and the chloride formed by reacting the aminothiophene with chloroacetyl chloride using Method A. The required thiol was prepared using Method L and reacted with the chloride using Method B described above. The ester was hydrolysed using Method C.

¹H NMR (400 MHz, CDCl₃) δ=12.56 (1H, s), 7.78 (2H, d, J=8.8 Hz), 7.32 (2H, d, J=8.8 Hz), 6.74 (1H, s), 3.74 (1H, d, J=16.5 Hz), 3.58 (1H, d, J=16.5 Hz), 3.34 (1H, dd, J=8.4 Hz), 2.75 (2H, dq, J=1.1 Hz, 7.3 Hz), 1.98-1.90 (1H, m), 1.85-1.77 (1H, m), 1.28 (3H, t, J=7.3 Hz), 1.03 (3H, t, J=7.3 Hz).

LCMS (Method A): R_{T}=12.72 min. m/z=476 (ES+, M+H), 474 (ES−, M−H)

### Example 128

### {[5-Ethyl-3-(4-trifluoromethoxy-benzoyl)-thiophen-2-ylcarbamoyl]-methylsulfanyl}-phenyl-acetic acid

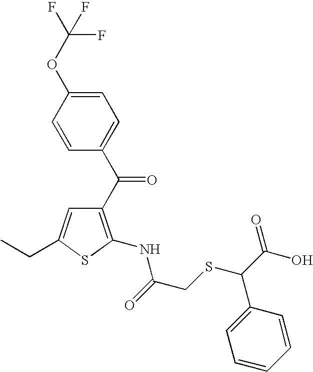

The required aminothiophene was prepared as described for Example 1 starting from (4-trifluoromethoxybenzoyl)acetonitrile and the chloride formed by reacting the aminothiophene with chloroacetyl chloride using Method A. The required thiol was prepared using Method L and reacted with the chloride using Method B described above. The ester was hydrolysed using Method C.

¹H NMR (400 MHz, CDCl₃) δ=12.42 (1H, s), 7.77 (2H, d, J=8.5 Hz), 7.46 (2H, d, J=8.5 Hz), 7.34-7.25 (5H, m), 4.83 (2H, s), 3.56 (1H, d, J=16.4 Hz), 3.43 (1H, d, J=16.4 Hz), 2.75 (2H, dq, J=1.1 Hz, 7.5 Hz), 1.28 (3H, t, J=7.5 Hz).

LCMS (Method A): R_{T}=13.05 min. m/z=524 (ES+, M+H), 522 (ES−, M−H)

### Example 129

### (S)-2-{[5-Ethyl-3-(4-trifluoromethoxy-benzoyl)-thiophen-2-ylcarbamoyl]-methylsulfanyl}-propionic acid

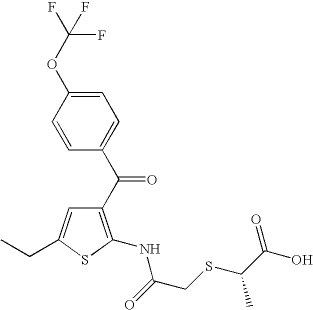

The required aminothiophene was prepared as described for Example 1 starting from (4-trifluoromethoxybenzoyl)acetonitrile and the chloride formed by reacting the aminothiophene with chloroacetyl chloride using Method A. The required thiol was prepared using Method M and reacted with the chloride using Method B described above. The ester was hydrolysed using Method C.

¹H NMR (400 MHz, CDCl₃) δ=12.60 (1H, s), 7.78 (2H, d, J=8.8 Hz), 7.32 (2H, d, J=8.8 Hz), 6.75 (1H, s), 3.82-3.56 (3H, m), 2.75 (2H, dq, J=1.1 Hz and 7.5 Hz), 1.53 (3H, d, J=7.5 Hz), 1.29 (3H, t, J=7.5 Hz).

LCMS (Method A): R_{T}=12.53 min. m/z=462 (ES+, M+H), 460 (ES−, M−H)

### Example 130

### {[3-(3-Chloro-4-isopropoxy-benzoyl)-5-ethyl-thiophen-2-ylcarbamoyl]-methylsulfanyl}-acetic acid

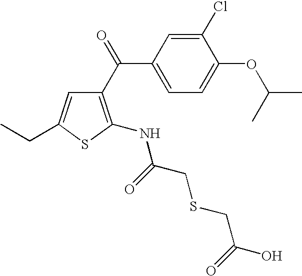

The title compound was prepared from (3-chloro-4-isopropoxybenzoyl)acetonitrile by an analogous procedure to Example 1.

¹H NMR (400 MHz, CDCl₃) δ=12.44 (1H, bs, 1H), 7.75 (1H, d, J=2 Hz), 7.58 (1H, dd, J=8, 2 Hz), 6.93 (1H, d, J=9 Hz), 6.75 (1H, s), 4.63 (1H, septet, J=6 Hz), 3.59 (2H, s), 3.36 (2H, s), 2.70 (2H, q, J=8 Hz), 1.37 (6H, d, J=6 Hz), 1.23 (3H, t, J=8 Hz).

LCMS (Method A): R_{T}=12.32 min. m/z 456/458 (ES+, M+H), 454/456 (ES−, M−H)

### Example 131

### {[5-Ethyl-3-(3-fluoro-4-trifluoromethoxy-benzoyl)-thiophen-2-ylcarbamoyl]-methylsulfanyl}-acetic acid

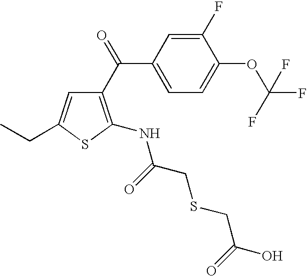

The title compound was prepared from (3-fluoro-4-trifluoromethoxybenzoyl)acetonitrile by an analogous procedure to Example 1.

¹H NMR (400 MHz, CDCl₃) δ=12.43 (1H, bs), 7.53 (1H, dd, J=10, 2 Hz), 7.48 (1H, ddd, J=8, 4, 1 Hz), 7.37 (1H, ddd, J=9, 7, 2 Hz), 6.68 (1H, t, J=1 Hz), 3.60 (2H, s), 3.36 (2H, s), 2.70 (2H, qd, J=8, 1 Hz), 1.26 (3H, t, J=8 Hz).

LCMS (Method B): R_{T}=11.25 min. m/z 466 (ES+, M+H), 464 (ES−, M−H)

### Example 132

### {[5-Propyl-3-(4-trifluoromethoxy-benzoyl)-thiophen-2-ylcarbamoyl]-methylsulfanyl}-acetic acid

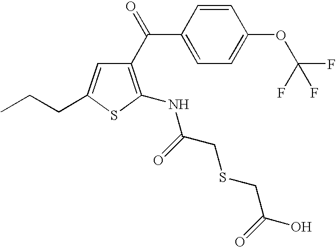

The title compound was prepared by an analogous procedure to Example 1, using 1-pentanal in place of butyraldehyde.
¹H NMR (400 MHz, CDCl₃) δ=12.46 (1H, bs), 7.72 (2H, d, J=9 Hz), 7.26 (2H, d, J=9 Hz), 6.69 (1H, t, J=1 Hz), 3.60 (2H, s), 3.37 (2H, s), 2.63 (2H, td, J=8, 1 Hz), 1.61 (2H, qt, J=8, 8 Hz), 0.90 (3H, t, J=8 Hz).

LCMS (Method B): R_{T}=11.76 min. m/z 462 (ES+, M+H), 460 (ES−, M−H)

### Example 133

### {[5-Isopropyl-3-(4-trifluoromethoxy-benzoyl)-thiophen-2-ylcarbamoyl]-methylsulfanyl}-acetic acid

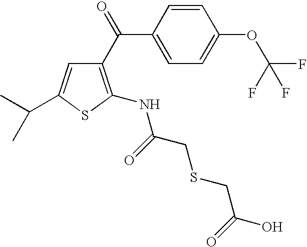

The title compound was prepared by an analogous procedure to Example 1, using 3-methyl-1-butanal in place of butyraldehyde.

¹H NMR (400 MHz, CDCl₃) δ=12.48 (1H, bs), 7.71 (2H, d, J=9 Hz), 7.26 (2H, bd, J=9 Hz), 6.69 (1H, d, J=1 Hz), 3.60 (2H, s), 3.37 (2H, s), 3.02 (1H, d, J=7, 1 Hz), 1.27 (6H, d, J=7 Hz).

LCMS (Method B): R_{T}=11.59 min. m/z 462 (ES+, M+H), 460.15 (ES−, M−H)

### Example 134

### {[5-sec-Butyl-3-(4-trifluoromethoxy-benzoyl)-thiophen-2-ylcarbamoyl]-methylsulfanyl}-acetic acid

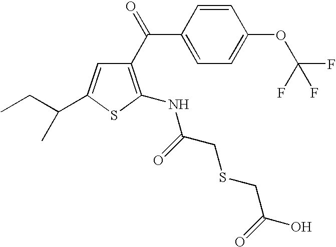

The title compound was prepared by an analogous procedure to Example 1, using 3-methyl-1-pentanal in place of butyraldehyde.

¹H NMR (400 MHz, CDCl₃) δ=12.49 (1H, bs), 7.72 (2H, d, J=9 Hz), 7.26 (2H, bd, J=9 Hz), 6.69 (1H, d, J=1 Hz), 3.60 (2H, s), 3.37 (2H, s), 2.75 (1H, qt, J=6, 6 Hz), 1.57 (2H, dq, J=8, 8 Hz), 1.23 (3H, d, J=8 Hz), 0.82 (3H, t, J=7 Hz).

LCMS (Method B): R_{T}=12.15 min. m/z 476 (ES+, M+H), 474 (ES−, M−H)

### Example 135

### (4-{[5-Ethyl-3-(4-fluoro-benzoyl)-thiophen-2-ylcarbamoyl]-methylsulfanyl}-phenoxy)-acetic acid

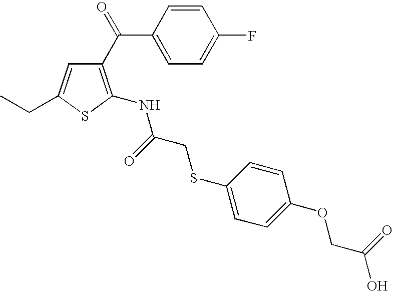

The title compound was prepared by an analogous procedure to Example 89, starting from (4-fluorobenzoyl)acetonitrile.

¹H NMR (400 MHz, DMSO-d6) δ=12.36 (1H, bs), 7.81 (2H, dd, J=9, 6 Hz), 7.41 (4H, d, J=8 Hz), 6.88 (2H, d, J=9 Hz), 6.82 (1H, t, J=1 Hz), 4.62 (2H, s), 4.04 (2H, s), 2.72 (2H, qd, J=8, 1 Hz), 1.20 (3H, t, J=8 Hz).

LCMS (Method B): R_{T}=11.81 min. m/z 474 (ES+, M+H), 472 (ES−, M−H)

### Example 136

### (4-{[3-(4-Chloro-benzoyl)-5-ethyl-thiophen-2-ylcarbamoyl]-methylsulfanyl}-phenoxy)-acetic acid

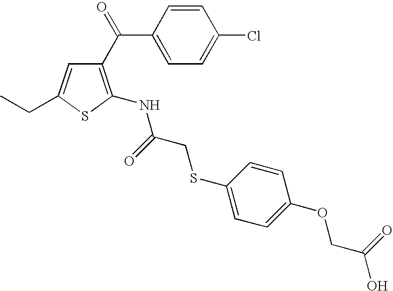

The title compound was prepared by an analogous procedure to Example 89, starting from (4-chlorobenzoyl)acetonitrile.

¹H NMR (400 MHz, DMSO-d6) δ=12.37 (1H, bs), 7.75 (2H, d, J=9 Hz), 7.63 (2H, d, J=9 Hz), 7.40 (2H, d, J=9 Hz), 6.88 (2H, d, J=9 Hz), 6.81 (1H, bs), 4.61 (2H, s), 4.05 (2H, s), 2.72 (2H, q, J=7 Hz), 1.19 (3H, t, J=7 Hz).

LCMS (Method A): R_{T}=12.49 min. m/z 490/492 (ES+, M+H), 488/490 (ES−, M−H)

### Example 137

### 2-(4-{[5-Ethyl-3-(4-fluoro-benzoyl)-thiophen-2-ylcarbamoyl]-methylsulfanyl}-phenoxy)-2-methyl-propionic acid

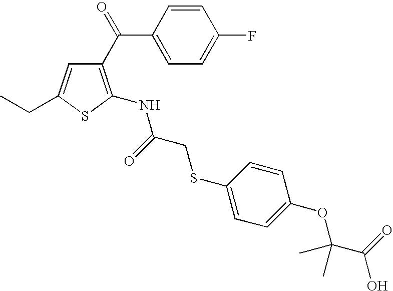

The title compound was prepared by an analogous procedure to Example 135, using tert-butyl-bromoisobutyrate in the step of Method F.

¹H NMR (400 MHz, DMSO-d6) δ=12.31 (1H, bs), 12.81 (2H, dd, J=9, 6 Hz), 7.41-7.35 (4H, m), 6.82 (1H, t, J=1 Hz), 6.77 (2H, d, J=9 Hz), 4.05 (2H, s), 2.72 (2H, qd, J=8, 1 Hz), 1.46 (6H, s), 1.20 (3H, t, J=8 Hz).

LCMS (Method B): R_{T}=11.83 min. m/z 502 (ES+, M+H), 500 (ES−, M−H)

### Example 138

### 2-(4-{[3-(4-Chloro-benzoyl)-5-ethyl-thiophen-2-ylcarbamoyl]-methylsulfanyl}-phenoxy)-2-methyl-propionic acid

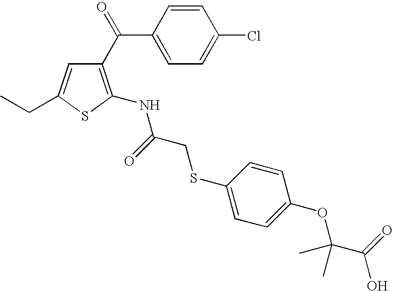

The title compound was prepared by an analogous procedure to Example 136, using tert-butyl-bromoisobutyrate in the step of Method F.

¹H NMR (400 MHz, DMSO-d6) δ=12.30 (1H, bs), 7.74 (2H, d, J=9 Hz), 7.63 (2H, d, J=9 Hz), 7.37 (2H, d, J=9 Hz), 6.81 (1H, t, J=1 Hz), 6.77 (2H, d, J=9 Hz), 4.05 (2H, s), 2.72 (2H, qd, J=8, 1 Hz), 1.46 (6H, s), 1.20 (3H, t, J=8 Hz).

LCMS (Method B): R_{T}=12.58 min. m/z 518/520 (ES+, M+H), 516/518 (ES−, M−H)

### Example 139

### {[3-(Benzothiazole-2-carbonyl)-5-ethyl-thiophen-2-ylcarbamoyl]-methylsulfanyl}-acetic acid

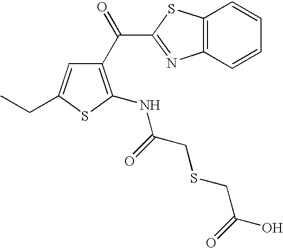

The title compound was prepared by an analogous procedure to Example 1, starting from 3-benzothiazol-2-yl-3-oxo-propionitrile.

¹H NMR (400 MHz, DMSO-d6) δ=12.48 (1H, bs), 8.348.27 (2H, m), 8.24 (1H, t, J=1 Hz), 3.79 (2H, s), 3.44 (2H, s), 2.84 (2H, qd, J=8, 1 Hz), 1.30 (3H, t, J=8 Hz).

LCMS (Method A): R_{T}=12.17 min. m/z 421 (ES+, M+H), 419 (ES−, M−H)

### Example 140

### {[3-(Benzofuran-2-carbonyl)-5-ethyl-thiophen-2-ylcarbamoyl]-methylsulfanyl}-acetic acid

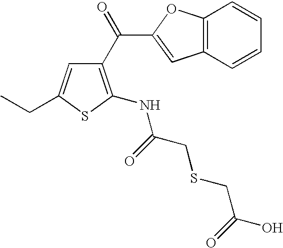

The title compound was prepared by an analogous procedure to Example 1, starting from 3-benzofuran-2-yl-3-oxo-propionitrile.

¹H NMR (400 MHz, DMSO-d6) δ=12.43 (1H, bs), 7.93 (1H, s), 7.88 (1H, d, J=8 Hz), 7.81 (1H, d, J=8 Hz), 7.61 (1H, s), 7.58 (2H, ddd, J=8, 7, 1 Hz), 7.41 (1H, dd, J=8, 1 Hz), 3.74 (2H, s), 3.42 (2H, s), 2.84 (2H, qd, J=7, 1 Hz), 1.30 (3H, t, J=7 Hz).

LCMS (Method A): R_{T}=11.47 min. m/z=404 (ES+, M+H), 402 (ES−, M−H)

### Example 141

### (4-{[5-Methyl-3-(4-trifluoromethoxy-benzoyl)-thiophen-2-ylcarbamoyl]-methylsulfanyl}-phenoxy)-acetic acid

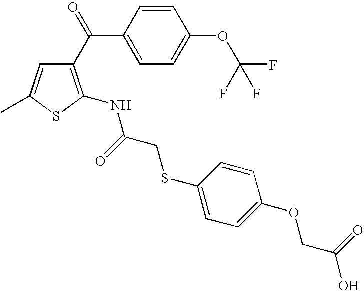

The title compound was prepared by an analogous procedure to Example 93, using propionaldehyde in place of butyraldehyde.

¹H NMR (400 MHz, DMSO-d6) δ=12.36 (1H, bs), 7.86 (2H, d, J=9 Hz), 7.55 (2H, bd, J=8 Hz), 7.41 (2H, d, J=9 Hz), 6.88 (2H, d, J=9 Hz), 6.82 (1H, d, J=1 Hz), 4.63 (2H, s), 4.05 (2H, s), 2.34 (3H, d, J=1 Hz).

LCMS (Method B): R_{T}=12.32 min. m/z 526 (ES+, M+H), 524 (ES−, M−H)

### Example 142

### {[3-(3-Chloro-4-trifluoromethoxy-benzoyl)-5-ethyl-thiophen-2-ylcarbamoyl]-methylsulfanyl}-acetic acid

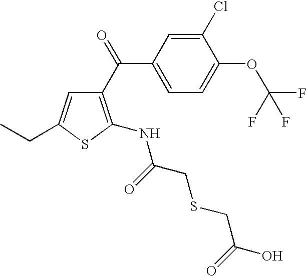

The title compound was prepared by an analogous procedure to Example 1, using (3-chloro-4-trifluoromethoxybenzoyl)acetonitrile.

¹H NMR (400 MHz, CDCl₃) δ=12.44 (1H, bs), 7.79 (1H, d, J=2 Hz), 7.59 (1H, dd, J=8, 2 Hz), 7.37 (1H, dd, J=8, 1 Hz), 6.67 (1H, t, J=1 Hz), 3.60 (2H, s), 3.35 (2H, s), 2.70 (2H, qd, J=7.1 Hz), 1.23 (3H, t, J=7 Hz).

LCMS (Method B): R_{T}=11.85 min. m/z 481/483 (ES+, M+H), 480/482 (ES−, M−H)

### Example 143

### {[5-Ethyl-3-(3-trifluoromethoxy-benzoyl)-thiophen-2-ylcarbamoyl]-methylsulfanyl}-acetic acid

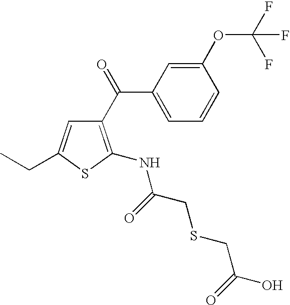

The title compound was prepared by an analogous procedure to Example 1, using (3-trifluoromethoxybenzoyl)acetonitrile.

¹H NMR (400 MHz, CDCl₃) δ=12.47 (1H, bs), 7.59 (1H, dt, J=8, 1 Hz), 7.51 (1H, bs), 7.47 (1H, t, J=8 Hz), 7.34 (1H, bd, J=8 Hz), 6.67 (1H, t, J=1 Hz), 3.60 (2H, s), 3.36 (2H, s), 2.68 (2H, qd, I=8, 1 Hz), 1.22 (3H, t, J=8 Hz).

LCMS (Method A): R_{T}=11.85 min. m/z 448 (ES+, M+H), 406 (ES−, M−H)

### Example 144

### {[3-(1,5-Dimethyl-1H-pyrazole-3-carbonyl)-5-ethyl-thiophen-2-ylcarbamoyl]-methylsulfanyl}-acetic acid

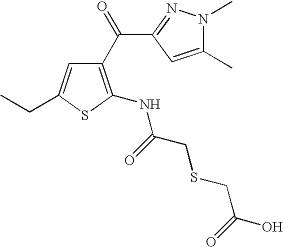

The title compound was prepared by an analogous procedure to Example 1, using 3-(1,5-dimethyl-1H-pyrazol-3-yl)-3-oxo-propionitrile.

¹H NMR (400 MHz, DMSO-d6) δ=12.65 (1H, bs), 7.97 (1H, t, J=1 Hz), 6.68 (1H, s), 3.89 (3H, s), 3.72 (2H, s), 3.40 (2H, s), 2.76 (2H, qd, J=8, 1 Hz), 2.33 (3H, s), 1.25 (3H, t, J=8 Hz).

LCMS (Method A): R_{T}=9.73 min. m/z 381 (ES+, M+H), 379 (ES−, M−H)

### Example 145

### {[5-Ethyl-3-(4-pyridin-2-yl-benzoyl)-thiophen-2-ylcarbamoyl]-methylsulfanyl}-acetic acid

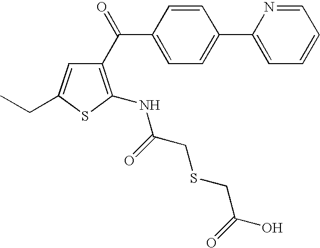

The title compound was prepared by an analogous procedure to Example 38, using 6-phenyl-2-pyridin-2-yl-[1,3,6,2]dioxazaborocane in the final coupling step.

¹H NMR (400 MHz, CDCl₃) δ=12.51 (1H, bs), 8.71 (1H, bs), 8.03 (2H, bd, J=7 Hz), 7.78 (4H, bd, J=8 Hz), 7.29 (1H, bs), 6.76 (1H, s), 3.61 (2H, s), 3.38 (2H, s), 2.68 (2H, q, J=8 Hz), 1.22 (3H, t, J=8 Hz).

LCMS (Method A): R_{T}=10.49 min. m/z 441 (ES+, M+H), 439 (ES−, M−H)

### Example 146

### 4-[5-Ethyl-3-(4-trifluoromethoxy-benzoyl)-thiophen-2-ylcarbamoyl]-2,2-dimethyl-butyric acid

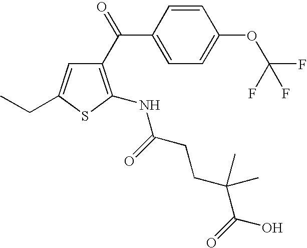

The title compound was prepared by an analogous procedure to Example 1, using 3,3-dimethyl-dihydro-pyran-2,6-dione in the final acylation step.

¹H NMR (400 MHz, CDCl₃) δ=11.88 (1H, bs), 7.69 (2H, d, J=9 Hz), 7.25 (2H, d, J=8 Hz), 6.65 (1H, t, J=1 Hz), 2.67 (2H, qd, J=8, 1 Hz), 2.53-2.48 (2H, m), 2.02-1.96 (2H, m), 1.22 (6H, s), 1.21 (3H, t, J=8 Hz).

LCMS (Method B): R_{T}=12.41 min. m/z 458 (ES+, M+H), 456 (ES−, M−H)

### Example 147

### 4-[3-(4-Chloro-benzoyl)-5-ethyl-thiophen-2-ylcarbamoyl]-2,2-dimethyl-butyric acid

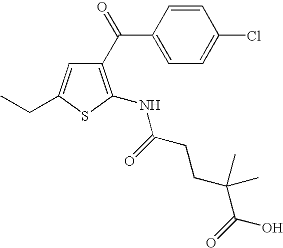

The title compound was prepared by an analogous procedure to Example 146, starting from (4-chlorobenzoyl)acetonitrile.

¹H NMR (400 MHz, CDCl₃) δ=11.88 (1H, bs), 7.58 (2H, d, J=8 Hz), 7.39 (2H, d, J=8 Hz), 6.34 (1H, t, J=1 Hz), 2.66 (2H, qd, J=8, 1 Hz), 2.53-2.87 (2H, m), 2.00-1.97 (2H, m), 1.21 (6H, s), 1.20 (3H, t, J=8 Hz).

LCMS (Method B): R_{T}=12.22 min. m/z 408/410 (ES+, M+H), 406/408 (ES−, M−H)

### Example 148

### 4-[5-Ethyl-3-(3-fluoro-4-trifluoromethoxy-benzoyl)-thiophen-2-ylcarbamoyl]-2,2-dimethyl-butyric acid

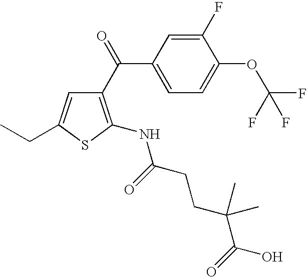

The title compound was prepared by an analogous procedure to Example 146, starting from (3-fluoro-4-trifluoromethoxybenzoyl)acetonitrile.

¹H NMR (400 MHz, CDCl₃) δ=11.82 (1H, bs), 7.49 (1H, dd, J=10, 10 Hz), 7.45 (1H, bd, J=10 Hz), 7.36 (1H, t, J=8 Hz), 6.63 (1H, t, J=1 Hz), 2.67 (2H, qd, J=7, 1 Hz), 2.55-2.48 (m, 2H), 2.03-1.96 (2H, m), 1.21 (6H, s), 1.21 (3H, t, J=7 Hz).

LCMS (Method B): R_{T}=12.51 min. m/z 476 (ES+, M+H), 474 (ES−, M−H)

### Example 149

### {[5-Ethyl-3-(1-methyl-1H-indole-2-carbonyl)-thiophen-2-ylcarbamoyl]-methylsulfanyl}-acetic acid

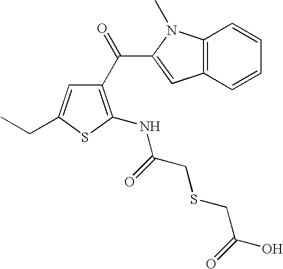

The title compound was prepared by an analogous procedure to Example 1, starting from 3-(1-methyl-1H-indol-2-yl)-3-oxo-propionitrile.

¹H NMR (400 MHz, DMSO-d6) δ=12.17 (1H, bs), 7.75 (1H, d, J=8 Hz), 7.61 (1H, d, J=8 Hz), 7.39 (1H, ddd, J=7, 7, 1 Hz), 7.19-7.14 (3H, m), 3.98 (3H, s), 3.73 (2H, s), 3.41 (2H, s), 2.78 (2H, qd, J=8, 1 Hz), 1.25 (3H, t, J=8 Hz).

LCMS (Method B): R_{T}=11.27 min. m/z 417 (ES+, M+H), 415 (ES−, M−H)

### Example 150

### {[5-Ethyl-3-(1-methyl-5-trifluoromethoxy-1H-indole-2-carbonyl)-thiophen-2-ylcarbamoyl]-methylsulfanyl}-acetic acid

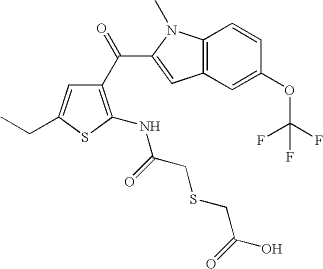

The title compound was prepared by an analogous procedure to Example 1, starting from 3-(1-methyl-5-trifluoromethoxy-1H-indol-2-yl)-3-oxo-propionitrile.

¹H NMR (400 MHz, DMSO-d6) δ=12.16 (1H, bs), 7.76 (1H, bs), 7.74 (1H, d, J=9 Hz), 7.36 (1H, dd, J=8, 2 Hz), 7.22 (1H, s), 7.16 (1H, t, J=1 Hz), 3.99 (3H, s), 3.74 (2H, s), 3.42 (2H, s), 2.78 (2H, qd, J=7.1 Hz), 1.25 (3H, t, J=7 Hz).

LCMS (Method B): R_{T}=12.27 min. m/z 501 (ES+, M+H), 499 (ES−, M−H)

### Example 151

### {[3-(5-Chloro-1-methyl-1H-indole-2-carbonyl)-5-ethyl-thiophen-2-ylcarbamoyl]-methylsulfanyl}-acetic acid

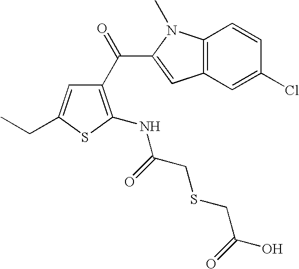

The title compound was prepared by an analogous procedure to Example 1, starting from 3-(5-chloro-1-methyl-1H-indol-2-yl)-3-oxo-propionitrile.

¹H NMR (400 MHz, CDCl₃) δ=12.42 (1H, bs), 7.61 (1H, bs), 7.27 (2H, bs), 7.02 (1H, t, J=1 Hz), 6.95 (1H, s), 3.92 (3H, s), 3.59 (2H, s), 3.37 (2H, s), 2.73 (2H, qd, J=7, 1 Hz), 1.25 (3H, t, J=7 Hz).

LCMS (Method B): R_{T}=12.09 min. m/z 450/452 (ES+, M+H), 449/451 (ES−, M−H)

### Example 152

### 4-[5-Ethyl-3-(1-methyl-5-trifluoromethoxy-H-indole-2-carbonyl)-thiophen-2-ylcarbamoyl]-2,2-dimethyl-butyric acid

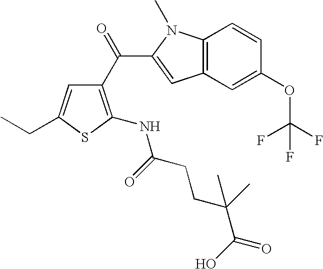

The title compound was prepared by an analogous procedure to Example 146, starting from 3-(1-methyl-5-trifluoromethoxy-1H-indol-2-yl)-3-oxo-propionitrile.

¹H NMR (400 MHz, CDCl₃) δ=11.88 (1H, bs), 7.49 (1H, s), 7.32 (1H, d, J=9 Hz), 7.18 (1H, d, J=9 Hz), 6.99 (2H, s), 3.91 (3H, s), 2.70 (2H, qd, J=7, 1 Hz), 2.55-2.50 (2H, m), 2.03-1.98 (2H, m), 1.24 (3H, t, J=7 Hz), 1.22 (6H, s).

LCMS (Method B): R_{T}=13.24 min. m/z 511 (ES+, M+H), 509 (ES−, M−H)

### Example 153

### {[5-Ethyl-3-(6-trifluoromethoxy-benzothiazole-2-carbonyl)-thiophen-2-ylcarbamoyl]-methylsulfanyl}-acetic acid

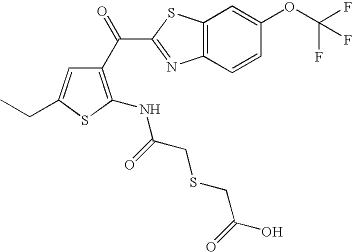

The title compound was prepared by an analogous procedure to Example 1, starting from 3-oxo-3-(6-trifluoromethoxy-benzothiazol-2-yl)-propionitrile.

¹H NMR (400 MHz, DMSO-d6) δ=12.45 (1H, bs), 8.44 (1H, s), 8.43 (1H, d, J=9 Hz), 8.20 (1H, t, J=1 Hz), 7.68 (1H, dd, J=8, 1 Hz), 3.80 (2H, s), 3.44 (2H, s), 2.83 (2H, qd, J=8, 1 Hz), 1.29 (3H, t, J=8 Hz).

LCMS (Method B): R_{T}=12.73 min. m/z 504 (ES+, M+H), 503 (ES−, M−H)

### Example 154

### {[3-(6-Chloro-benzothiazole-2-carbonyl)-5-ethyl-thiophen-2-ylcarbamoyl]-methylsulfanyl}-acetic acid

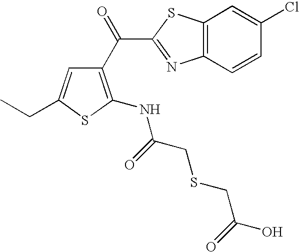

The title compound was prepared by an analogous procedure to Example 1, starting from 3-oxo-3-(6-chlorobenzothiazol-2-yl)-propionitrile.

¹H NMR (400 MHz, DMSO-d6) δ=12.46 (1H, bs), 8.46 (1H, d, J=2 Hz), 8.33 (1H, d, J=9 Hz), 8.20 (1H, t, J=1 Hz), 8.72 (1H, dd, J=9, 2 Hz), 3.79 (2H, s), 3.43 (2H, s), 2.83 (2H, qd, i=8, 1 Hz), 1.29 (3H, t, J=8 Hz).

LCMS (Method B): R_{T}=12.78 min. m/z 455/457 (ES+, M+H), 453/455 (ES−, M−H)

### Example 155

### {[3-(5-Chloro-benzothiazole-2-carbonyl)-5-ethyl-thiophen-2-ylcarbamoyl]-methylsulfanyl}-acetic acid

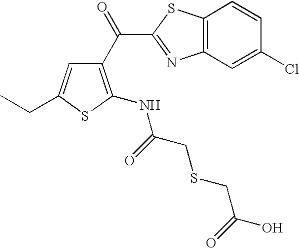

The title compound was prepared by an analogous procedure to Example 1, starting from 3-oxo-3-(5-chlorobenzothiazol-2-yl)-propionitrile.

¹H NMR (400 MHz, DMSO-d6) δ=12.44 (1H, bs), 8.45 (1H, d, J=2 Hz), 8.33 (1H, d, J=9 Hz), 8.20 (1H, t, J=1 Hz), 7.71 (1H, dd, J=9, 2 Hz), 3.79 (2H, s), 3.44 (2H, s), 2.83 (2H, qd, J=8, 1 Hz), 1.30 (3H, t, J=8 Hz).

LCMS (Method B): R_{T}=12.93 min. m/z 455/457 (ES+, M+H), 453/455 (ES−, M−H)

### Example 156

### {[3-(6-Chloro-quinoline-2-carbonyl)-5-ethyl-thiophen-2-ylcarbamoyl]-methylsulfanyl}-acetic acid

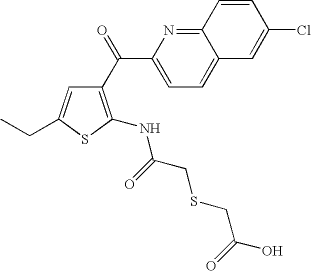

The title compound was prepared by an analogous procedure to Example 1, starting from 3-(6-chloro-quinolin-2-yl)-3-oxo-propionitrile.

¹H NMR (400 MHz, DMSO-d6) δ=12.53 (1H, bs), 8.60 (1H, d, J=8 Hz), 8.29 (1H, d, J=2 Hz), 8.22 (1H, d, J=9 Hz), 8.12 (1H, d, J=8 Hz), 7.91 (1H, dd, J=9, 2 Hz), 7.67 (1H, t, J=1 Hz), 3.78 (2H, s), 3.42 (2H, s), 2.77 (2H, qd, J=8, 1 Hz), 1.25 (3H, t, J=8 Hz).

LCMS (Method D): R_{T}=12.80 min. m/z 449/451 (ES+, M+H), 447/449 (ES−, M−H)

### Example 157

### {[3-(5-Chloro-1-methyl-1H-indole-3-carbonyl)-5-ethyl-thiophen-2-ylcarbamoyl]-methylsulfanyl}-acetic acid

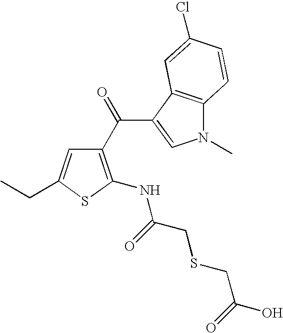

The title compound was prepared by an analogous procedure to Example 1, starting from 3-(5-chloro-1-methyl-1H-indol-3-yl)-3-oxo-propionitrile.

¹H NMR (400 MHz, DMSO-d6) δ=12.25 (1H, bs), 8.36 (1H, s), 8.28 (1H, d, J=2 Hz), 8.64 (1H, d, J=9 Hz), 7.35 (1H, dd, J=9, 2 Hz), 7.29 (1H, t, J=1 Hz), 3.93 (3H, s), 3.70 (2H, s), 3.42 (2H, s), 2.81 (2H, qd, J=7, 1 Hz), 1.29 (3H, t, J=7 Hz).

LCMS (Method D): R_{T}=10.83 min. m/z=451/453 (ES+, M+H), 449/451 (ES−, M−H)

### Example 158

### {1-[5-Ethyl-3-(4-trifluoromethoxy-benzoyl)-thiophen-2-ylcarbamoyl]-ethylamino}-acetic acid

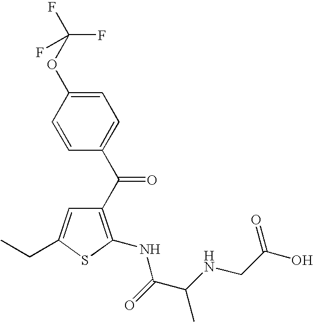

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.82 (1H, bs), 9.61 (1H, bs), 7.88 (2H, d, J=8.8 Hz), 7.57 (2H, d, J=8.8 Hz), 6.87 (1H, t, J=1.0 Hz), 4.44 (1H, bs), 3.88 (2H, m), 3.75-3.54 (2H, m), 2.78 (2H, qd, J=7.5, 1.0 Hz), 1.49 (3H, d, J=6.9 Hz), 1.23 (3H, t, J=7.5 Hz)

LCMS (Method A): R_{T}=9.14 min. m/z=345 (ES+, M+H), 343 (ES−, M−H)

### Example 159

### 1-{[5-Ethyl-3-(4-trifluoromethoxy-benzoyl)-thiophen-2-ylcarbamoyl]-methyl}-piperidine-3-carboxylic acid

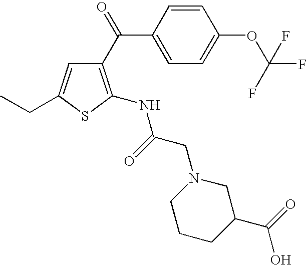

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.84 (1H, bs), 10.48 (1H, bs), 7.87 (2H, d, J=8.7 Hz), 7.56 (2H, d, J=8.7 Hz), 6.86 (1H, s), 4.47 (2H, m), 4.0-3.5 (3H, m), 3.3-2.8 (2H, m), 2.77 (2H, q, J=7.5 Hz), 2.05 (1H, m), 2.00-1.70 (2H, m), 1.55-1.40 (1H, m), 1.22 (3H, t, J=7.5 Hz)

LCMS (Method A): R_{T}=10.64 min. m/z=485 (ES+, M+H), 483 (ES−, M−H)

### Example 160

### (S)-2-({[5-Ethyl-3-(4-trifluoromethoxy-benzoyl)-thiophen-2-ylcarbamoyl]-methyl}-amino)-propionic acid

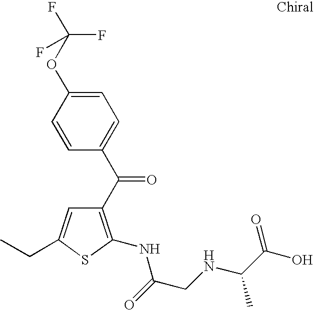

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.84 (1H, bs), 9.50 (1H, bs), 7.87 (2H, d, J=8.8 Hz), 7.56 (2H, m), 6.85 (1H, t, J=1.0 Hz), 4.28 (2H, m), 4.04 (1H, m), 2.77 (2H, qd, J=7.5, 1.0 Hz), 1.48 (3H, d, J=7.3 Hz), 1.22 (3H, t, J=7.5 Hz)

LCMS (Method A): R_{T}=9.12 min. m/z=445 (ES+, M+H), 443 (ES−, M−H)

### Example 161

### (R)-2-({[5-Ethyl-3-(4-trifluoromethoxy-benzoyl)-thiophen-2-ylcarbamoyl]-methyl}-amino)-propionic acid

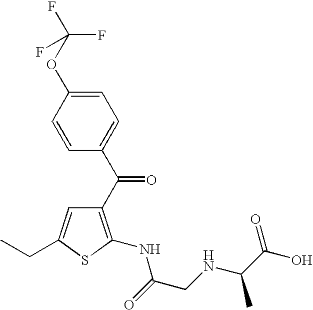

The required aminothiophene was prepared as described for Example 1 starting from (4-trifluoromethoxybenzoyl)acetonitrile. The acid bearing side chain was introduced by Methods A, D and E respectively, as described above.
¹H NMR (400 MHz, DMSO-d6) δ=11.84 (1H, bs), 9.50 (1H, bs), 7.87 (2H, d, J=8.8 Hz), 7.56 (2H, m), 6.86 (1H, t, J=1.0 Hz), 4.28 (2H, m), 4.05 (1H, m), 2.77 (2H, qd, J=7.5, 1.0 Hz), 1.48 (3H, d, J=7.3 Hz), 1.22 (3H, t, J=7.5 Hz)

LCMS (Method A): R_{T}=9.10 min. m/z=445 (ES+, M+H), 443 (ES−, M−H)

### Example 162

### 1-{[5-Ethyl-3-(4-trifluoromethoxy-benzoyl)-thiophen-2-ylcarbamoyl]-methyl}-pyrrolidine-3-carboxylic acid

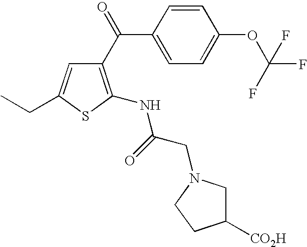

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.83 (1H, s), 10.81 (1H, bs), 7.87 (2H, d, J=8.7 Hz), 7.56 (2H, d, J=8.7 Hz), 6.86 (1H, s), 4.54 (2H, m), 4.0-3.6 (2H, m), 3.6-3.1 (3H, m), 2.77 (2H, q, J=7.5 Hz), 2.4-2.1 (2H, m), 1.22 (3H, t, J=7.5 Hz)

LCMS (Method A): R_{T}=7.89 min. m/z=471 (ES+, M+H), 469 (ES−, M−H)

### Example 163

### 1-{[5-Ethyl-3-(4-trifluoromethoxy-benzoyl)-thiophen-2-ylcarbamoyl]-methyl}-piperidine-4-carboxylic acid

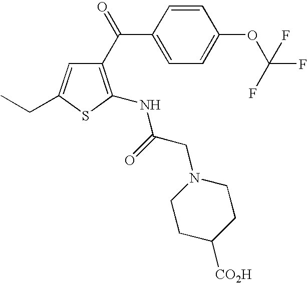

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.82 (1H, m), 10.34 (1H, bs), 7.87 (2H, d, J=8.7 Hz), 7.56 (2H, d, J=8.7 Hz), 6.87 (1H, s), 4.49 (2H, m), 3.59 (2H, m), 3.3-3.1 (2H, m), 2.81 (2H, q, J=7.5 Hz), 2.2-2.0 (3H, m), 2.0-1.85 (2H, m), 1.26 (3H, t, J=7.5 Hz)

LCMS (Method A): R_{T}=8.82 min. m/z=485 (ES+, M+H), 483 (ES−, M−H)

### Example 164

### 1-({[5-Ethyl-3-(4-trifluoromethoxy-benzoyl)-thiophen-2-ylcarbamoyl]-methyl}-amino)-cyclobutanecarboxylic acid

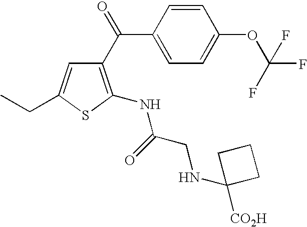

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.48 (1H, bs), 9.93 (1H, bs), 7.87 (2H, d, J=8.8 Hz), 7.56 (2H, m), 6.85 (1H, t, J=1.0 Hz), 4.25 (2H, bs), 2.77 (2H, qd, J=7.5, 1.0 Hz), 2.54 (2H, m), 2.43 (2H, m), 2.04 (2H, m), 1.22 (3H, t, J=7.5 Hz)

LCMS (Method A): R_{T}=11.27 min. m/z=471 (ES+, M+H), 469 (ES−, M−H)

### Example 165

### 1-{[5-Ethyl-3-(4-trifluoromethoxy-benzoyl)-thiophen-2-ylcarbamoyl]-methyl}-piperidine-2-carboxylic acid

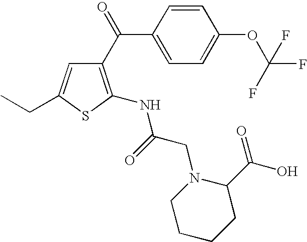

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=7.85 (2H, d, J=8.2 Hz), 7.54 (2H, d, J=8.1 Hz), 6.83 (1H, s), 4.0 (2H, m, obscured), 3.7-3.4 (2H, m), 3.2-2.9 (1H, m), 2.75 (2H, q, J=7.5 Hz), 2.15-1.87 (2H, m), 1.80-1.64 (2H, m), 1.63-1.35 (2H, m), 1.21 (3H, t, J=7.5 Hz)

LCMS (Method A): R_{T}=12.68 min. m/z=485 (ES+, M+H), 483 (ES−, M−H)

### Example 166

### 1-({[5-Ethyl-3-(4-trifluoromethoxy-benzoyl)-thiophen-2-ylcarbamoyl]-methyl}-amino)-cyclohexanecarboxylic acid

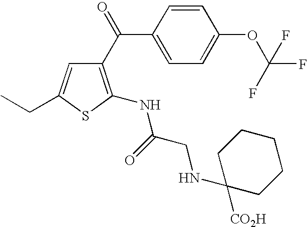

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.80 (1H, bs), 9.5 (2H, bs), 7.87 (2H, d, J=8.8 Hz), 7.56 (2H, d, J=8.8 Hz), 6.85 (1H, m), 4.3 (2H, bs), 2.76 (2H, q, J=7.5 Hz), 2.10 (2H, m), 1.9-1.6 (4H, m), 1.6-1.4 (3H, m), 1.35-1.25 (1H, m), 1.22 (3H, t, J=7.5 Hz)

LCMS (Method C): R_{T}=9.97 min. m/z=499 (ES+, M+H), 497 (ES−, M−H)

### Example 167

### (S)-1-{[5-Ethyl-3-(4-trifluoromethoxy-benzoyl)-thiophen-2-ylcarbamoyl]-methyl}-pyrrolidine-2-carboxylic acid

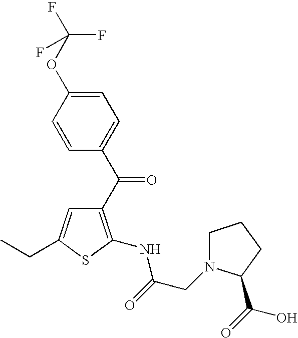

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.98 (1H, bs), 7.86 (2H, d, J=8.8 Hz), 7.56 (2H, m), 6.85 (1H, t, J=1.0 Hz), 4.6-4.2 (3H, m), 3.61 (1H, m), 3.18 (1H, m), 2.76 (2H, qd, J=7.5, 1.0 Hz), 2.38 (1H, m), 2.05 (2H, m), 1.93 (1H, m), 1.22 (3H, t, J=7.5 Hz)

LCMS (Method C): R_{T}=6.06 min. m/z=471 (ES+, M+H), 469 (ES−, M−H)

### Example 168

### (R)-1-{[5-Ethyl-3-(4-trifluoromethoxy-benzoyl)-thiophen-2-ylcarbamoyl]-methyl}-pyrrolidine-2-carboxylic acid

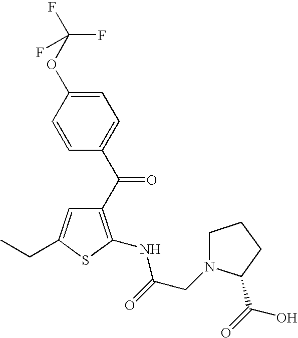

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=11.98 (1H, bs), 7.86 (2H, d, J=8.8 Hz), 7.56 (2H, m), 6.85 (1H, t, J=1.0 Hz), 4.6-4.2 (3H, m), 3.60 (1H, m), 3.17 (1H, m), 2.76 (2H, qd, J=7.5, 1.0 Hz), 2.38 (1H, m), 2.05 (2H, m), 1.93 (1H, m), 1.22 (3H, t, J=7.5 Hz)

LCMS (Method B): R_{T}=8.80 min. m/z=471 (ES+, M+H), 469 (ES−, M−H)

### Example 169

### (S)-2-({[3-(3-Chloro-4-trifluoromethoxy-benzoyl)-5-ethyl-thiophen-2-ylcarbamoyl]-methyl}-amino)-3-methyl-butyric acid

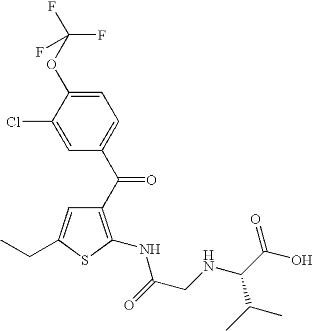

The required aminothiophene was prepared as described for Example 1 starting from (3-chloro-4-trifluoromethoxybenzoyl)acetonitrile. The acid bearing side chain was introduced by Methods A, D and E respectively, as described above.

¹H NMR (400 MHz, DMSO-d6) δ=11.83 (1H, bs), 9.46 (1H, bs), 7.96 (1H, d, J=1.9 Hz), 7.80 (1H, dd, J=8.4, 1.9 Hz), 7.76 (1H, m), 6.85 (1H, m), 4.25 (2H, bs), 3.8 (1H, obscured), 2.76 (2H, qd, J=7.5, 0.9 Hz), 2.31 (1H, m), 1.22 (3H, t, J=7.5 Hz), 1.07 (3H, d, J=7.0 Hz), 1.02 (3H, d, J=7.0 Hz)

LCMS (Method B): R_{T}=12.16 min. m/z=507/509 (ES+, M+H), 505/507 (ES−, M−H)

### Example 170

### (S)-2-({[3-(3,4-Dichloro-benzoyl)-5-ethyl-thiophen-2-ylcarbamoyl]-methyl}-amino)-3—methyl-butyric acid

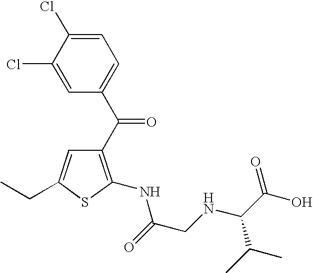

The required aminothiophene was prepared as described for Example 1 starting from (3,4-dichlorobenzoyl)acetonitrile. The acid bearing side chain was introduced by Methods A, D and E respectively, as described above.

¹H NMR (400 MHz, DMSO-d6) δ=11.81 (1H, bs), 9.46 (1H, bs), 7.89 (1H, d, J=2.0 Hz), 7.84 (1H, d, J=8.3 Hz), 7.67 (1H, dd, J=8.3, 2.0 Hz), 6.84 (1H, t, J=1.0 Hz), 4.24 (2H, bs), 3.83 (1H, bs), 2.75 (2H, qd, J=7.5, 1.0 Hz), 2.32 (1H, m), 1.22 (3H, t, J=7.5 Hz), 1.07 (3H, d, J=6.9 Hz), 1.02 (3H, d, J=6.9 Hz)

LCMS (Method B): R_{T}=11.88 min. m/z=457/459/461 (ES+, M+H), 455/457/459 (ES−, M−H)

### Example 171

### 2-({[3-(3-Chloro-4-trifluoromethoxy-benzoyl)-5-ethyl-thiophen-2-ylcarbamoyl]-methyl}-amino)-2-methyl-propionic acid

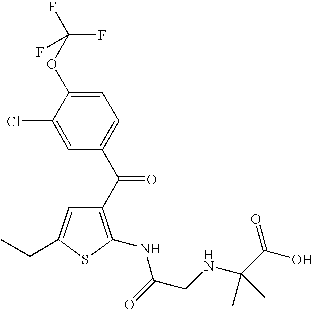

The required aminothiophene was prepared as described for Example 1 starting from (3-chloro-4-trifluoromethoxybenzoyl)acetonitrile. The acid bearing side chain was introduced by Methods A, D and E respectively, as described above.

¹H NMR (400 MHz, DMSO-d6) δ=11.81 (1H, bs), 9.61 (1H, bs), 7.98 (1H, d, J=2.0 Hz), 7.81 (1H, dd, J=8.5, 2.0 Hz), 7.76 (1H, m), 6.87 (1H, s), 4.33 (2H, bs), 2.77 (2H, qd, J=7.5, 0.9 Hz), 1.52 (6H, s), 1.22 (3H, t, J=7.5 Hz)

LCMS (Method B): R_{T}=9.71 min. m/z=495/493 (ES+, M+H), 493/491 (ES−, M−H)

### Example 172

### (R)-1-{[5-Ethyl-3-(4-trifluoromethoxy-benzoyl)-thiophen-2-ylcarbamoyl]-methyl}-2-methyl-pyrrolidine-2-carboxylic acid

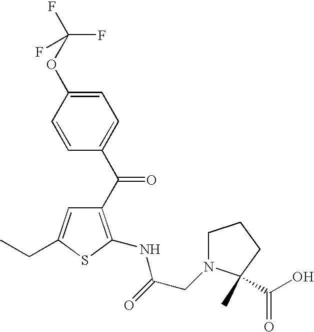

The required aminothiophene was prepared as described for Example 1 starting from (4-trifluoromethoxybenzoyl)acetonitrile.

2-Methyl-D-proline was prepared by a literature procedure. (A. K. Beck et al. Organic Syntheses, Coll. Vol. 9, p. 626 (1998); Vol. 72, p. 62 (1995))

The acid bearing side chain was introduced by Methods A, D and E respectively, as described above.

¹H NMR (400 MHz, DMSO-d6) δ=12.36 (1H, bs), 7.85 (2H, d, J=8.8 Hz), 7.55 (2H, d, J=8.7 Hz), 6.84 (1H, s), 5.0-4.3 (3H, m), 3.35-3.1 (1H, m), 2.75 (2H, q, J=7.5 Hz), 2.29 (1H, m), 2.1-1.8 (3H, m), 1.46 (3H, bs), 1.21 (3H, t, J=7.5 Hz)

LCMS (Method B): R_{T}=11.63 min. m/z=485 (ES+, M+H), 483 (ES−, M−H)

### Example 173

### 1-{[5-Ethyl-3-(4-trifluoromethoxy-benzoyl)-thiophen-2-ylcarbamoyl]-methyl}-2-methyl-pyrrolidine-2-carboxylic acid

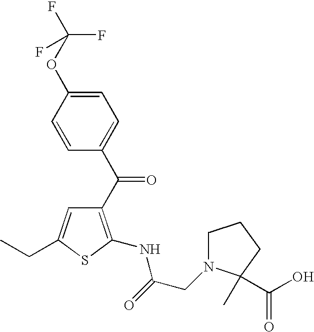

The required aminothiophene was prepared as described for Example 1 starting from (4-trifluoromethoxybenzoyl)acetonitrile. The acid bearing side chain was introduced by Methods A, D and E respectively, as described above.

¹H NMR (400 MHz, DMSO-d6) δ=12.3 (1H, bs), 7.85 (2H, d, J=8.8 Hz), 7.55 (2H, d, J=8.7 Hz), 6.84 (1H, s), 5.4-4.4 (3H, m), 3.35-3.0 (1H, m), 2.75 (2H, qd, J=7.5, 0.8 Hz), 2.29 (1H, m), 2.1-1.8 (3H, m), 1.46 (3H, bs), 1.21 (3H, t, J=7.5 Hz)

LCMS (Method B): R_{T}=11.55 min. m/z=485 (ES+, M+H), 483 (ES−, M−H)

### Example 174

### 4-[5-Ethyl-3-(4-trifluoromethoxy-benzoyl)-thiophen-2-ylcarbamoyl]-3-methyl-butyric acid

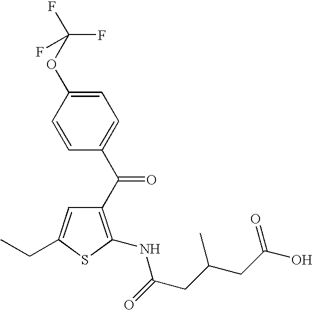

The title compound was prepared by an analogous procedure to Example 1, using 4-methylglutaric anhydride in the final acylation step.

¹H NMR (400 MHz, CDCl₃) δ=11.91 (1H, bs), 7.70 (2H, d, J=8.8 Hz), 7.26 (2H, m), 6.66 (1H, t, J=1.1 Hz), 2.68 (2H, qd, J=7.5, 1.1 Hz), 2.64-2.38 (4H, m), 2.32 (1H, dd, J=15.5, 6.8 Hz), 1.21 (3H, t, J=7.5 Hz), 1.07 (3H, d, J=6.6 Hz)

LCMS (Method B): R_{T}=11.84 min. m/z=444 (ES+, M+H), 442 (ES−, M−H)

### Example 175

### ({5-Ethyl-3-[4-(2,2,2-trifluoro-ethoxy)-benzoyl]-thiophen-2-ylcarbamoyl}-methylsulfanyl)-acetic acid

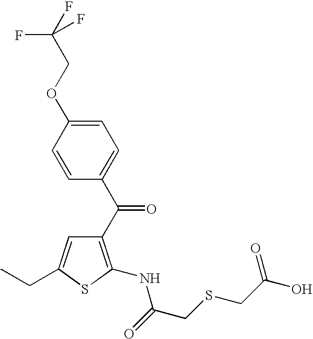

The title compound was prepared by an analogous procedure to Example 1, starting from 4-(2,2,2-trifluoroethoxy)benzoyl acetonitrile.

¹H NMR (400 MHz, CDCl₃) δ=12.49 (1H, s), 7.70 (2H, d, J=8.8 Hz), 6.96 (2H, d, J=8.8 Hz), 6.72 (1H, t, J=1.0 Hz), 4.37 (2H, q, J=8.0 Hz), 3.59 (2H, s), 3.35 (2H, s), 2.69 (2H, qd, J=7.5, 1.0 Hz), 1.22 (3H, t, J=7.5 Hz)

LCMS (Method A): R_{T}=11.49 min. m/z=462 (ES+, M+H), 460 (ES−, M−H)

### Example 176

### {3-[5-Ethyl-3-(4-trifluoromethoxy-benzoyl)-thiophen-2-ylcarbamoyl]-piperidin-1-yl}-acetic acid

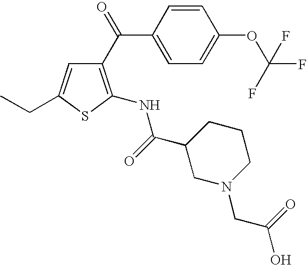

The required aminothiophene was prepared as described for Example 1 starting from (4-trifluoromethoxybenzoyl)acetonitrile, and reacted with 1-(benzyloxycarbonyl)-piperidine-3-carbonyl chloride via Method A.

The benzyloxycarbonyl group was removed as follows.

3-[5-Ethyl-3-(4-trifluoromethoxy-benzoyl)-thiophen-2-ylcarbamoyl]-piperidine-1-carboxylic acid benzyl ester (266 mg, 0.48 mmol) is dissolved in acetic acid (3 ml), treated with 33% HBr/AcOH (3 mL) and stirred for 30 minutes. The solution is diluted with water (20 ml) and extracted twice with dichloromethane (30 ml). The combined organic extracts are washed with brine (3×50 ml), dried over sodium sulphate, filtered and evaporated. The residual yellow gum is repeatedly triturated in diethyl ether/petroleum ether providing the desired piperidine hydrobromide salt as a yellow powder (230 mg, 95% yield).

The synthesis is completed via Methods F and E, as described above.

¹H NMR (400 MHz, DMSO-d6) δ=11.80 (1H, bs), 7.86 (2H, d, J=8.8 Hz), 7.56 (2H, m), 6.84 (1H, t, J=1.0 Hz), 4.12 (2H, m), 3.63 (1H, m), 3.55-3.15 (3H, m), 3.04 (1H, m), 2.75 (2H, qd, J=7.5, 1.0 Hz), 2.06 (1H, m), 1.90 (2H, m), 1.60 (1H, m), 1.21 (3H, t, J=7.5 Hz)

LCMS (Method A): R_{T}=9.03 min. m/z=485 (ES+, M+H), 483 (ES−, M−H)

### Example 177

### {4-[5-Ethyl-3-(4-trifluoromethoxy-benzoyl)-thiophen-2-ylcarbamoyl]-piperidin-1-yl}-acetic acid

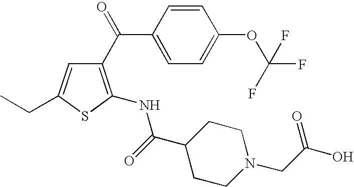

The title compound was prepared by an analogous procedure to Example 176, using 1-(benzyloxycarbonyl)-piperidine-4-carbonyl chloride in the synthesis of the side chain.

¹H NMR (400 MHz, DMSO-d6) δ=11.78 (1H, s), 7.85 (2H, d, J=8.8 Hz), 7.56 (2H, d, J=8.8 Hz), 6.84 (1H, t, J=1.0 Hz), 4.12 (2H, s), 3.55 (2H, m), 3.14 (2H, m), 2.94 (1H, m), 2.74 (2H, qd, J=7.5, 1.0 Hz), 2.12 (2H, m), 2.00 (2H, m), 1.21 (3H, t, J=7.5 Hz)

LCMS (Method A): R_{T}=8.95 min. m/z=485 (ES+, M+H), 483 (ES−, M−H)

### Example 178

### (2R*,5R*)-1-{[5-Ethyl-3-(4-trifluoromethoxy-benzoyl)-thiophen-2-ylcarbamoyl]-methyl}-5-methyl-pyrrolidine-2-carboxylic acid

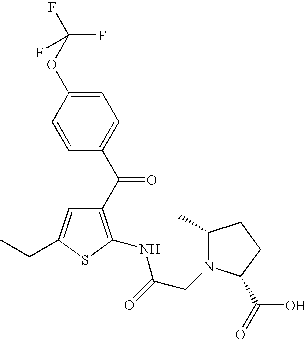

The required aminothiophene was prepared as described for Example 1 starting from (4-trifluoromethoxybenzoyl)acetonitrile. The side chain was introduced using Methods A and D respectively.

Racemic cis-5-methylproline methyl ester was prepared by a literature method (C. G. Overberger et al. Macromolecules, p. 368, Vol. 5(4), 1972)

The methyl ester was hydrolysed as follows.

### Method N

Hydrolysis of methyl and ethyl esters of basic amine containing examples.

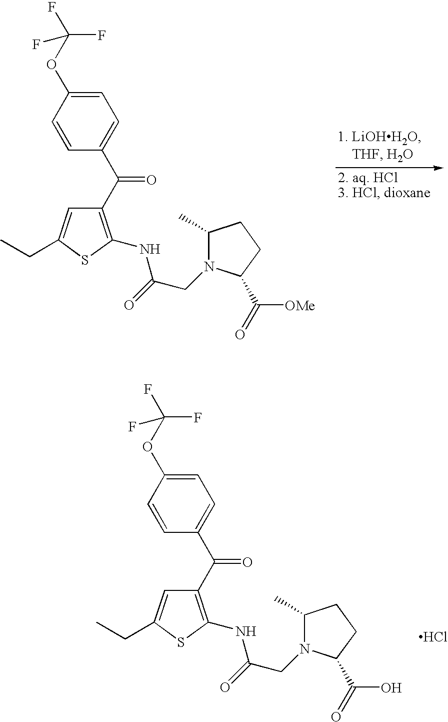

A solution of the methyl ester (144 mg, 0.29 mmol) in tetrahydrofuran (3 ml) and water (2 ml) is treated with lithium hydroxide monohydrate (12.2 mg, 0.29 mmol) and stirred at room temperature. After 3 hours 60% conversion was observed by LC-MS and further with lithium hydroxide monohydrate (12.2 mg, 0.29 mmol) added.

After 4.5 hours total 1 M aqueous HCl (1 ml) is added and the solution extracted with diethyl ether (5 ml). The ethereal extract is dried over sodium sulphate, filtered and evaporated. The residual yellow solid is the free base form of the desired product.

Treatment with a solution of hydrogen chloride in diethyl ether or dioxane, followed by removal of solvent in vacuo and trituration in diethyl ether/petroleum ether provides the desired product as the hydrochloride salt (90 mg, 60% yield).

¹H NMR (400 MHz, DMSO-d6) δ=12.19 (1H, bs), 7.85 (2H, d, J=8.8 Hz), 7.55 (2H, d, J=8.8 Hz), 6.83 (1H, s), 4.5-3.7 (4H, obscured), 2.75 (2H, q, j=7.5 Hz), 2.25 (1H, bs), 2.05 (2H, bs), 1.61 (1H, bs), 1.24 (3H, m), 1.21 (3H, t, J=7.5 Hz)

LCMS (Method A): R_{T}=11.31 min. m/z=485 (ES+, M+H), 483 (ES−, M−H)

### Example 179

### (2R*,5S*)-1-{[5-Ethyl-3-(4-trifluoromethoxy-benzoyl)-thiophen-2-ylcarbamoyl]-methyl}-5-methyl-pyrrolidine-2-carboxylic acid

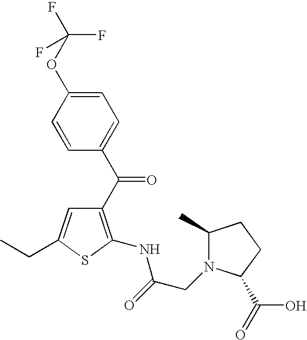

The required aminothiophene was prepared as described for Example 1 starting from (4-trifluoromethoxybenzoyl)acetonitrile. The side chain was introduced using Methods A and D respectively. The methyl ester was hydrolysed via Method N.

Racemic trans-5-methylproline methyl ester was prepared by a literature method (C. G. Overberger et al. Macromolecules, p. 368, Vol. 5(4), 1972)

¹H NMR (400 MHz, DMSO-d6) δ=12.1 (1H, bs), 7.86 (2H, d, J=8.8 Hz), 7.55 (2H, d, J=8.7 Hz), 6.84 (1H, s), 4.8-4.1 (2H, m, obscured), 3.9-3.5 (2H, bs), 2.75 (2H, q, J=7.5 Hz), 2.41 (1H, m), 2.15 (1H, m), 1.98 (1H, bs), 1.70 (1H, bs), 1.21 (3H, m, obscured), 1.21 (3H, t, J=7.5 Hz)

LCMS (Method B): R_{T}=11.85 min. m/z=485 (ES+, M+H), 483 (ES−, M−H)

### Example 180

### 1-{[5-Ethyl-3-(4-trifluoromethoxy-benzoyl)-thiophen-2-ylcarbamoyl]-methyl}-4-methyl-pyrrolidine-2-carboxylic acid

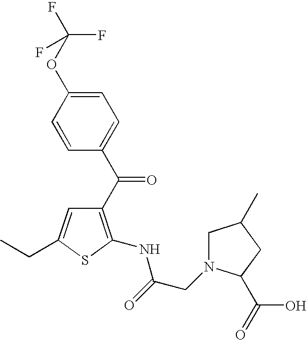

The required aminothiophene was prepared as described for Example 1 starting from (4-trifluoromethoxybenzoyl)acetonitrile. The side chain was introduced using Methods A and D respectively. The methyl ester was hydrolysed via Method N.

4-Methylproline methyl ester was prepared as a mixture of diastereoisomers by a literature method (Burgstahler et al. Nature p. 388, Vol 202 (1964)

¹H NMR (400 MHz, DMSO-d6) δ=12.08 (1H, bs), 7.92 (2H, d, J=8.0 Hz), 7.62 (2H, d, J=8.0 Hz), 6.91 (1H, s), 4.8-4.0 (4H, obscured), 3.7 (2H, m), 3.5-3.1 (2H, m), 2.82 (2H, q, J=7.5 Hz), 2.5-2.35 (0.5H, m), 2.35-2.2 (0.5H, m), 2.15-2.0 (0.5H, m), 1.8-1.65 (0.5H, m), 1.28 (3H, t, J=7.5 Hz), 1.13 (3H, m)

LCMS (Method B): R_{T}=10.14 and 10.21 min. m/z=485 (ES+, M+H), 483 (ES−, M−H)

### Example 181

### (2R,5R)-1-{[5-Ethyl-3-(4-trifluoromethoxy-benzoyl)-thiophen-2-ylcarbamoyl]-methyl}-5-(4-fluoro-phenyl)-pyrrolidine-2-carboxylic acid

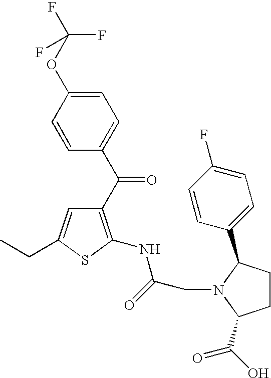

The required aminothiophene was prepared as described for Example 1 starting from (4-trifluoromethoxybenzoyl)acetonitrile.

The side chain was introduced using Methods A and D respectively. The ethyl ester was hydrolysed via Method N.

The required D-trans-5-(4-fluorophenyl)-proline ethyl ester was prepared as follows, by a literature procedure (I. Collado et al. J. Org. Chem. p. 5011, Vol. 60 (1995))

### Step 1: Boc-D-Pyr-OEt

A solution of D-Pyr-OEt (5.1 g, 32.6 mmol) in dichloromethane (70 ml) is treated with triethylamine (4.55 ml, 32.6 mmol), di-tert-butyl-dicarbonate (14.2 g, 65.2 mmol) and 4-(dimethylamino)-pyridine (3.98 g, 32.6 mmol) and the resulting yellow solution stirred at room temperature. After 1.5 h, TLC analysis showed complete conversion. The solution is washed with water twice, then brine, dried over sodium sulphate, filtered and evaporated. The residue is purified by column chromatography (1:1 ethyl acetate/petroleum ether) affording Boc-D-Pyr-OEt as a viscous light yellow oil (7.98 g, 95% yield) which solidifies on standing.

Step 2: Boc-D-5-hydroxyproline ethyl ester

A solution of Boc-D-Pyr-OEt (7.95 g, 31 mmol) in tetrahydrofuran (200 mL) is cooled at −78° C. under a nitrogen atmosphere and treated dropwise with a 1M solution of lithium triethylborohydride in tetrahydrofuran (37.2 mL, 37.2 mmol) over 20 minutes. After 30 minutes at −76° C. the reaction is quenched at this temperature with saturated aqueous sodium bicarbonate (80 mL) and the mixture allowed to warm to 0° C. 35% aqueous hydrogen peroxide (8 mL) is added, resulting in dissolution of the precipitate. After 30 minutes the solution is extracted with diethyl ether (3×200 mL). The combined extracts are washed with water and brine, dried over sodium sulphate, filtered and evaporated. The desired product is obtained as a clear, colourless gum (7.88 g, 98% yield).

Step 3: Boc-D-5-methoxypyrroline ethyl ester

A solution of Boc-D-5-hydroxyproline ethyl ester (7.87 g, 30.4 mmol) in methanol (100 ml) is treated with p-toluenesulfonic acid monohydrate (571 mg, 3.0 mmol) and the solution stirred at room temperature over night. Saturated aqueous sodium bicarbonate (20 ml) is added and the mixture stirred for 10 minutes. The methanol is removed under vacuum and the residue partitioned between water (100 ml) and diethyl ether (100 ml). The aqueous phase is extracted with further diethyl ether (2×100 ml) and the combined organic phases washed with brine, dried over sodium sulphate, filtered and evaporated. The desired hemiaminal is obtained as a pale yellow gum (7.48 g, 90% yield).

Step 4: Boc-D-trans-5-(4-fluorophenyl)proline ethyl ester

A suspension of copper (I) bromide-dimethylsulfide complex (1.64 g, 8 mmol, 4 equiv) in dry diethyl ether (16 ml) is cooled at −40° C. under nitrogen and treated dropwise with a 0.8 M solution of 4-fluorophenylmagnesium bromide in tetrahydrofuran (10 ml, 8 mmol, 4 equiv). The yellow suspension is stirred at −40° C. for 45 minutes and cooled to −75° C., before dropwise addition of boron trifluoride diethyl etherate (1.01 ml, 8 mmol, 4 equiv). After 30 minutes at −76° C., a solution of Boc-D-5-methoxypyrroline ethyl ester (546 mg, 2 mmol, 1 equiv) in diethyl ether (3 ml) is added dropwise, and the suspension stirred for 15 minutes before warming to room temperature over 3 hours. After 1 h at room temperature the mixture is quenched with a 1:1 mixture of saturated aqueous ammonium chloride/ammonium hydroxide (25 ml) and stirred for 30 minutes. The aqueous phase is extracted with diethyl ether (2×100 ml) and the combined organic phases washed with water and saturated aqueous sodium bicarbonate, dried over sodium sulfate, filtered and evaporated.

The crude material is purified by column chromatography (9:1 petroleum ether/ethyl acetate), providing the desired product as a clear, colourless oil (566 mg, 81% yield).

Step 5: D-trans-5-(4-fluorophenyl)proline ethyl ester

A solution of Boc-D-trans-5-(4-fluorophenyl)proline ethyl ester (560 mg, 1.66 mmol) in dichloromethane (30 ml) is treated with trifluoroacetic acid (1.5 ml, 20 mmol) and stirred at room temperature for 2 h. The solvent is removed under vacuum and the residue dissolved in dichloromethane (50 ml). The solution is washed with saturated aqueous sodium bicarbonate (2×5 ml) and the combined aqueous phases extracted with dichloromethane (3×50 ml). The combined organic extracts are dried over sodium sulphate, filtered and evaporated.

The crude material is purified by column chromatography (3% methanol in dichloromethane providing the desired product as a clear, colourless oil (319 mg, 81% yield).

¹H NMR (400 MHz, DMSO-d6) δ=12.50 (1H, bs), 7.90 (2H, d, J=8.8 Hz), 7.57 (2H, m), 7.49 (2H, m), 7.05 (2H, m), 6.81 (1H, t, J=1.0 Hz), 4.31 (1H, m), 4.08 (1H, d, J=7.5 Hz), 3.60 (1H, d, J=17.8 Hz), 3.38 (1H, d, J=17.8 Hz), 2.71 (2H, qd, J=7.5, 1.0 Hz), 2.57 (1H, m), 2.38 (1H, m), 2.05 (1H, m), 1.86 (1H, m), 1.18 (3H, t, J=7.5 Hz)

LCMS (Method B): R_{T}=11.16 min. m/z=565 (ES+, M+H), 563 (ES−, M−H)

### Example 182

### (2R,5S)-1-{[5-Ethyl-3-(4-trifluoromethoxy-benzoyl)-thiophen-2-ylcarbamoyl]-methyl}-5-methyl-pyrrolidine-2-carboxylic acid

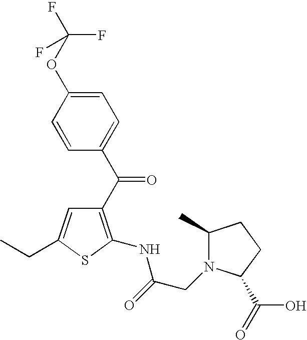

The title compound was prepared by an analogous procedure to Example 181, using D-trans-5-methylproline ethyl ester.

¹H NMR (400 MHz, DMSO-d6) δ=7.86 (2H, d, J=8.7 Hz), 7.55 (2H, d, J=8.6 Hz), 6.83 (1H, s), 4.8-4.1 (2H, m, obscured), 4.0-3.5 (2H, bs), 2.75 (2H, q, J=7.5 Hz), 2.40 (1H, m), 2.14 (1H, m), 1.97 (1H, m), 1.68 (1H, m), 1.21 (3H, t, J=7.5 Hz), 1.26-1.14 (3H, m, obscured)

LCMS (Method B): R_{T}=11.89 min. m/z=485 (ES+, M+H), 483 (ES−, M−H)

### Example 183

### (2R,5S)-5-Ethyl-1-{[5-ethyl-3-(4-trifluoromethoxy-benzoyl)-thiophen-2-ylcarbamoyl]-methyl}-pyrrolidine-2-carboxylic acid

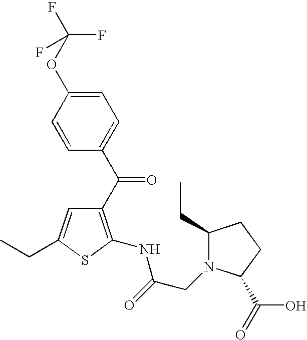

The title compound was prepared by an analogous procedure to Example 181, using D-trans-5-ethylproline ethyl ester.

¹H NMR (400 MHz, DMSO-d6) δ=12.5 (1H, bs), 7.85 (2H, d, J=8.7 Hz), 7.54 (2H, d, J=8.7 Hz), 6.83 (1H, s), 5.2-4.1 (2H, obs), 4.2-3.5 (2H, m), 2.74 (2H, q, J=7.5 Hz), 2.46-2.29 (1H, m), 2.20-2.04 (1H, m), 2.03-1.88 (1H, m), 1.80-1.60 (2H, m), 1.45-1.25 (1H, m), 1.21 (3H, t, J=7.5 Hz), 0.87 (3H, m)

LCMS (Method C): R_{T}=10.62 min. m/z=499 (ES+, M+H), 497 (ES−, M−H)

### Example 184

### (2R,5R)-1-{[5-Ethyl-3-(4-trifluoromethoxy-benzoyl)-thiophen-2-ylcarbamoyl]-methyl}-5-phenyl-pyrrolidine-2-carboxylic acid

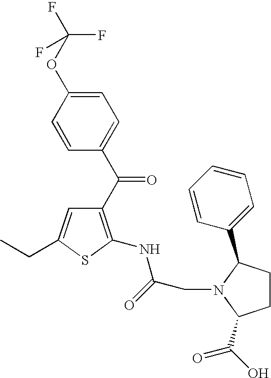

The title compound was prepared by an analogous procedure to Example 181, using D-trans-5-phenylproline ethyl ester.

¹H NMR (400 MHz, DMSO-d6) δ=12.51 (1H, bs), 7.90 (2H, d, J=8.4 Hz), 7.56 (2H, d, J=8.3 Hz), 7.45 (2H, d, J=7.3 Hz), 7.22 (3H, m), 6.81 (1H, s), 4.29 (1H, m), 4.09 (1H, m), 3.59 (1H, d, J=17.8 Hz), 3.38 (1H, m), 2.71 (2H, q, J=7.4 Hz), 2.56 (1H, m), 2.39 (1H, m), 2.05 (1H, m), 1.88 (1H, m), 1.18 (3H, t, J=7.4 Hz)

LCMS (Method C): R_{T}=11.08 min. m/z=547 (ES+, M+H), 545 (ES−, M−H)

### Example 185

### (2R,5R)-1-{[5-Ethyl-3-(4-trifluoromethoxy-benzoyl)-thiophen-2-ylcarbamoyl]-methyl}-5-isopropyl-pyrrolidine-2-carboxylic acid

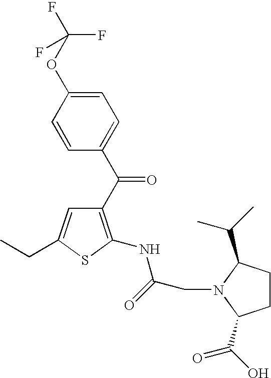

The title compound was prepared by an analogous procedure to Example 181, using D-trans-5-isopropylproline ethyl ester.

¹H NMR (400 MHz, DMSO-d6) δ=12.45 (1H, bs), 7.83 (2H, d, J=8.7 Hz), 7.53 (2H, d, J=8.7 Hz), 6.82 (1H, s), 4.0-3.5 (3H, m), 3.20 (1H, m), 2.74 (2H, q, J=7.5 Hz), 2.23 (1H, m), 1.98-1.73 (3H, m), 1.73-1.63 (1H, m), 1.20 (3H, t, J=7.5 Hz), 0.85 (6H, m)

LCMS (Method C): R_{T}=13.59 min. m/z=513 (ES+, M+H), 511 (ES−, M−H)

### Example 186

### (2R,5S)-1-{1-[5-Ethyl-3-(4-trifluoromethoxy-benzoyl)-thiophen-2-ylcarbamoyl]-ethyl}-5-methyl-pyrrolidine-2-carboxylic acid

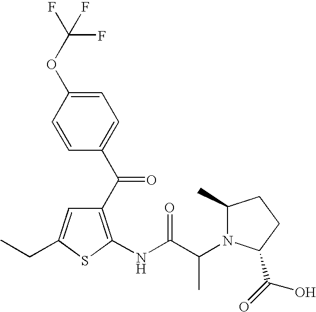

The title compound was prepared by an analogous procedure to Example 181, using 2-chloropropionyl chloride in Method A and D-trans-5-methylproline ethyl ester in Method D.

¹H NMR (400 MHz, CDCl₃) δ=12.39 and 12.33 (1H, 2×bs), 7.83 and 7.79 (2H, 2×d, J=8.4 Hz), 7.34 and 7.29 (2H, 2×d, J=8.4 Hz), 6.81 and 6.75 (1H, 2×s), 5.28-5.10 (2H, bm), 4.83-4.71 (1H, bm), 4.53-4.39 (1H, bm), 4.25-4.15 (1H, bm), 2.83-2.70 (3H, m), 2.36-2.19 (2H, bm), 1.90-1.83 (2H, bm), 1.68-1.61 and 1.53-1.43 (3H, 2×m), 1.33-1.26 (3H, m)

LCMS (Method C): R_{T}=12.36 and 12.83 min. m/z=499 (ES+, M+H), 497 (ES−, M−H)

### Example 187

### (2R,5R)-1-{[3-(4-Chloro-benzoyl)-5-ethyl-thiophen-2-ylcarbamoyl]-methyl}-5-phenyl-pyrrolidine-2-carboxylic acid hydrochloride

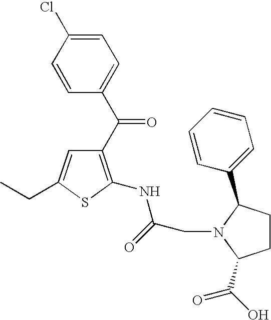

The title compound was prepared by an analogous procedure to Example 181, starting from using (4-chlorobenzoyl)acetonitrile, and using D-trans-5-isopropylproline ethyl ester in Method D.

¹H NMR (400 MHz, DMSO-d6) δ=12.50 (1H, bs), 7.78 (2H, d, J=8.5 Hz), 7.65 (2H, d, J=8.5 Hz), 7.45 (2H, m), 7.22 (3H, m), 6.79 (1H, m), 4.29 (1H, m), 4.09 (1H, m), 3.59 (1H, d, J=17.8 Hz), 3.38 (1H, m), 2.70 (2H, q, J=7.5 Hz), 2.58 (1H, m), 2.39 (1H, m), 2.06 (1H, m), 1.89 (1H, m), 1.18 (3H, t, J=7.5 Hz)

LCMS (Method C): R_{T}=11.11 min. m/z=499/497 (ES+, M+H), 497/495 (ES−, M−H)

### Example 188

### (2R,5R)-1-{[5-Chloro-3-(4-trifluoromethoxy-benzoyl)-thiophen-2-ylcarbamoyl]-methyl}-5-phenyl-pyrrolidine-2-carboxylic acid hydrochloride

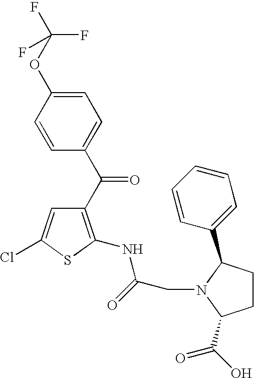

The required aminothiophene was prepared as in Example 65. The side chain was introduced according to Methods A, D and N.

D-trans-5-phenylproline ethyl ester was prepared as in Example 181.

¹H NMR (400 MHz, DMSO-d6) δ=12.53 (1H, s), 7.93 (2H, d, J=8.8 Hz), 7.58 (2H, m), 7.41 (2H, m), 7.21 (3H, m), 7.16 (1H, s), 4.27 (1H, dd, J=8.2, 6.0 Hz), 4.12 (1H, dd, J=8.2, 1.7 Hz), 3.68 (1H, d, J=18.0 Hz), 3.44 (1H, d, J=18.0 Hz), 2.57 (1H, m), 2.39 (1H, m), 2.07 (1H, m), 1.89 (1H, m)

LCMS (Method C): R_{T}=11.26 min. m/z=553/555 (ES+, M+H), 551/553 (ES−, M−H)

### Example 189

### 1-{[5-Ethyl-3-(4-trifluoromethoxy-benzoyl)-thiophen-2-ylcarbamoyl]-methyl}-pyrrolidine-2-carboxylic acid

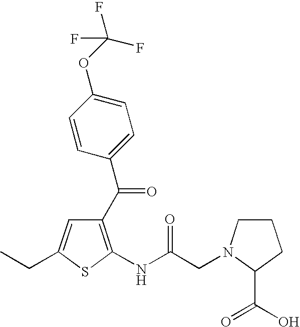

The required aminothiophene was prepared as in Example 1. The side chain was introduced according to Methods A, D and N.

¹H NMR (400 MHz, CD₃OD) δ=12.48 (1H, s), 7.67 (2H, d, J=7.6 Hz), 7.23 (2H, d, J=7.6 Hz), 6.63 (1H, app. t, J=1.0 Hz), 3.74 (1H, d, 17.2 Hz), 3.59 (2H, 1H, dd, J=4.6 Hz), 3.54 (1H, d, J=17.2 Hz), 3.24 (1H, ddd, J=4.4, 7.2, 11.6 Hz), 2.71-2.64 (3H, m), 2.31-2.21 (1H, m), 2.13-2.10 (1H, m), 1.95-1.87 (2H, m), 1.20 (3H, t, J=7.2 Hz)

LCMS (Method A): R_{T}=9.87 min. m/z=471.22 (ES+, M+H), 469.28 (ES−, M−H)

### Example 190

### 1-({[5-Ethyl-3-(4-trifluoromethoxy-benzoyl)-thiophen-2-ylcarbamoyl]-methyl}-amino)-cyclopentanecarboxylic acid

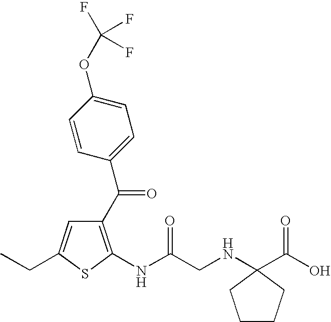

The required aminothiophene was prepared as in Example 1. The side chain was introduced according to Methods A, D and N.

¹H NMR (400 MHz, CD₃OD) δ=12.48 (1H, s), 7.83 (2H, d, J=7.2 Hz), 7.54 (2H, d, J=7.6 Hz), 6.80 (1H, s), 3.39 (2H, s), 2.73 (2H, q, J=7.4 Hz), 1.97-1.89 (4H, m), 1.70-1.64 (4H, m), 1.20 (3H, t, J=7.6 Hz)

LCMS (Method A): R_{T}=11.58 min. m/z=485 (ES+, M+H), 483 (ES−, M−H)

### Example 191

### (S)-2-({[5-Ethyl-3-(4-trifluoromethoxy-benzoyl)-thiophen-2-ylcarbamoyl]-methyl}-amino)-butyric acid

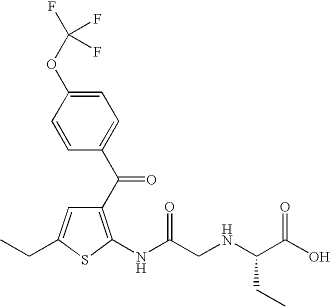

The required aminothiophene was prepared as in Example 1. The side chain was introduced according to Methods A, D and N.

¹H NMR (400 MHz, CD₃OD) δ=7.89 (2H, d, J=8.2 Hz), 7.46 (2H, d, J=8.0 Hz), 6.87 (1H, s), 4.39 (1H, d, J=16.4 Hz), 4.34 (1H, d, J=16.4 Hz), 4.12 (1H, t, I=6.4 Hz), 2.81 (2H, q, J=7.6 Hz), 2.16-2.05 (2H, m), 1.31 (3H, t, J=7.6 Hz), 1.13 (3H, t, J=7.6 Hz).

LCMS (Method A): R_{T}=10.23 min. m/z=459 (ES+, M+H), 457 (ES−, M−H)

### Example 192

### (S)-2-({[5-Ethyl-3-(4-trifluoromethoxy-benzoyl)-thiophen-2-ylcarbamoyl]-methyl}-amino)-2-methyl-butyric acid

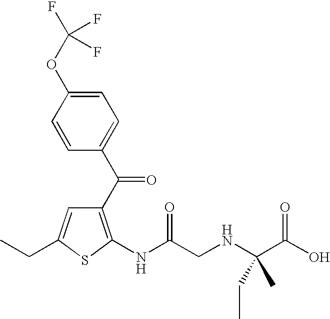

The required aminothiophene was prepared as in Example 1. The side chain was introduced according to Methods A, D and N.

¹H NMR (400 MHz, CD₃OD) δ=7.88 (2H, d, J=7.6 Hz), 7.47 (2H, d, J=7.6 Hz), 6.85 (1H, s), 4.02-3.80 (2H, m), 2.69 (2H, q, J=6.8 Hz), 1.94-1.78 (2H, m), 1.45-1.30 (3H, m), 1.19 (3H, t, J=7.2 Hz), 0.97-0.91 (3H, m).

LCMS (Method A): R_{T}=10.91 min. m/z=473 (ES+, M+H), 471 (ES−, M−H)

### Example 193

### (R)-1-{2-[5-Ethyl-3-(4-trifluoromethoxy-benzoyl)-thiophen-2-ylcarbamoyl]-ethyl}-pyrrolidine-2-carboxylic acid

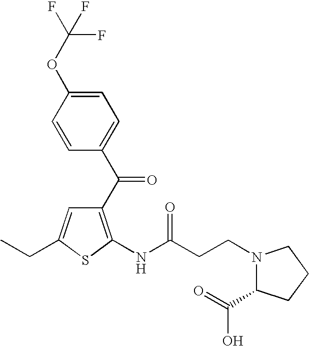

The required aminothiophene was prepared as in Example 1. The side chain was introduced according to Methods A, D and E. 3-Bromo-propionyl chloride was used in Method A.

¹H NMR (400 MHz, CDCl₃) δ=11.91 (1H, s), 7.69 (2H, d, J=8.9 Hz), 7.29 (2H, d, J=8.9 Hz), 6.63 (1H, s), 4.56-4.47 (1H, br. m), 4.17-4.09 (1H, br. m), 3.82-3.71 (1H, br. m), 3.54-3.46 (2H, br. m), 3.29-3.19 (1H, br. m), 2.73-2.60 (3H, br. m), 2.94-2.37 (1H, br. m), 1.22 (3H, t, J=7.3 Hz)

LCMS (Method A): R_{T}=9.11 min. m/z=485 (ES+, M+H), 483 (ES−, M−H)

### Example 194

### {2-[5-Ethyl-3-(4-trifluoromethoxy-benzoyl)-thiophen-2-ylcarbamoyl]-ethylsulfanyl}-acetic acid

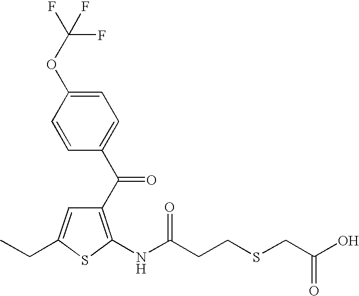

The required aminothiophene was prepared as in Example 1. The side chain was introduced according to Methods A, B and C. 3-Bromo-propionyl chloride was used in Method A.

¹H NMR (400 MHz, CDCl₃) δ=12.00 (1H, bs), 7.76 (2H, d, J=8.8 Hz), 7.33 (2H, d, J=8.8 Hz), 6.73 (1H, t, J=1.0 Hz), 3.34 (2H, s), 3.10 (2H, t, J=7.2 Hz), 2.90 (2H, t, J=7.2 Hz), 2.75 (2H, qd, J=7.2, 1.0 Hz), 1.28 (3H, t, J=7.2 Hz).

LCMS (Method A): R_{T}=12.26 min. m/z=462 (ES+, M+H), 460 (ES−, M−H)

### Example 195

### {(S)-1-[5-Ethyl-3-(4-trifluoromethoxy-benzoyl)-thiophen-2-ylcarbamoyl]-ethylsulfanyl}-acetic acid

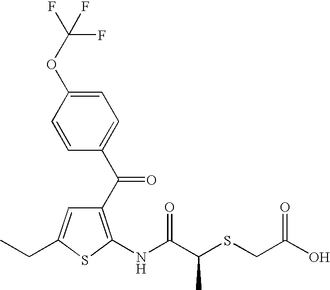

The required aminothiophene was prepared as in Example 1. The side chain was introduced by Method A, using (R)-2-bromopriopionyl chloride, then methods B and C, as described previously.

¹H NMR (400 MHz, CDCl₃) δ=12.54 (1H, s), 7.78 (2H, d, J=8.5 Hz), 7.32 (2H, d, J=8.5 Hz), 6.75 (1H, t, J=1.2 Hz), 3.90 (1H, q, J=7.0 Hz), 3.45 (1H, d, J=15.5 Hz), 3.35 (1H, d, J=15.5 Hz), 2.75, (2H, qd, J=7.5, 1.2 Hz), 1.63 (3H, d, J=7.0 Hz), 1.28 (3H, t, J=7.5 Hz)

LCMS (Method A): R_{T}=12.58 min. m/z=462 (ES+, M+H), 460 (ES−, M−H)

### Example 196

### 2-({[5-Ethyl-3-(4-trifluoromethoxy-benzoyl)-thiophen-2-ylcarbamoyl]-methyl}-methyl-amino)-2-methyl-propionic acid

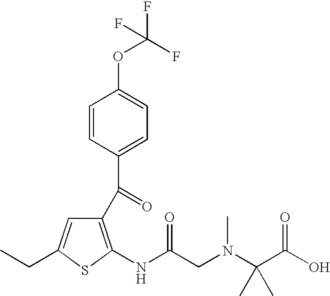

The required aminothiophene was prepared as in Example 1. The side chain was introduced by the following steps. Method A, using chloroacetyl chloride, Method D using tert-butyl-2-aminoisobutyrate, Method K, alkylation with iodomethane, and finally Method E.

¹H NMR (400 MHz, CDCl₃) δ=12.13 (1H, s), 7.60 (2H, d, J=8.2 Hz), 7.20 (2H, d, J=8.2 Hz), 6.59 (1H, s), 4.35 (1H, bs), 2.95 (3H, s), 2.65 (2H, q, J=7.2 Hz), 1.60 (6H, bs), 1.19 (3H, t, J=7.2 Hz).

LCMS (Method A): R_{T}=11.80 min. m/z=473 (ES+, M+H), 471 (ES−, M−H)

### Example 197

### 1-({[5-Ethyl-3-(4-trifluoromethoxy-benzoyl)-thiophen-2-ylcarbamoyl]-methyl}-methyl-amino)-cyclopropanecarboxylic acid

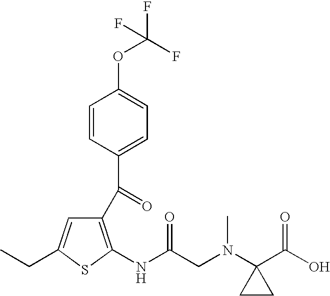

The required aminothiophene was prepared as in Example 1. The side chain was introduced by the following steps. Method A, using chloroacetyl chloride, Method D using tert-butyl-1-aminocyclopropane-1-carboxylate, Method K, alkylation with iodomethane, and finally Method E.

LCMS (Method A): R_{T}=12.88 min. m/z=471 (ES+, M+H), 469 (ES−, M−H)

### Example 198

### (S)-2-({[5-Ethyl-3-(4-trifluoromethoxy-benzoyl)-thiophen-2-ylcarbamoyl]-methyl}-methyl-amino)-propionic acid

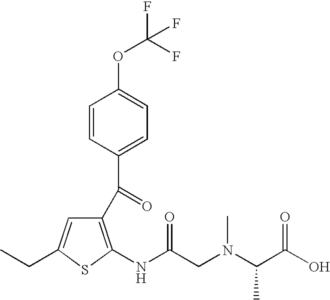

The required aminothiophene was prepared as in Example 1. The side chain was introduced by the following steps. Method A, using chloroacetyl chloride, Method D using alanine tert-butyl ester, Method K, alkylation with iodomethane, and finally Method E.

¹H NMR (400 MHz, CDCl₃) δ=12.74 (1H, s), 7.77 (2H, d, J=8.4 Hz), 7.32 (2H, d, J=8.4 Hz), 6.71 (1H, s), 3.60 (1H, d, J=17.5 Hz), 3.51 (1H, q, J=6.9 Hz), 3.46 (1H, d, J=17.5 Hz), 2.75 (2H, q, J=6.8 Hz), 2.54 (3H, s), 1.42 (3H, d, J=6.9 Hz), 1.28 (3H, t, J=6.8 Hz).

LCMS (Method A): R_{T}=11.61 min. m/z=459 (ES+, M+H), 457 (ES−, M−H) Biological Assays

### Biological Assay 1: Transactivation Assay

Compounds were screened for their functional potency in transiently transfected HEK293 cells for their ability to activate PPAR subtypes. Cells were cultured in DMEM (Invitrogen) supplemented with 10% foetal calf serum, glutamine, penicillin and streptomycin and plated at 10 000 cells/well of a 96-well solid white plate and incubated at 37° C./5% CO₂ for 24 hours. Media was removed and the cells washed with PBS. Cells were then transiently transfected using Fugene (Roche) with 50 ng pFACMV-PPARδ (plasmid encoding amino acids 1-147 of the GAL4 DNA binding domain, fused to amino acids 147-441 of PPARδ downstream of CMV promoter) and 250 ng pFR-Luc (reporter plasmid containing 5×GAL4 response elements upstream of a luciferase gene), using a ratio of 3:1 Fugene:DNA. 100 μl of this transfection mixture in DMEM (without foetal calf serum) was added to each well, and the incubation continued for a further 24 hours. The cells are then again washed with PBS prior to the addition of 100 μl reduced serum medium (OptiMEM; Invitrogen). Compounds were added (10 μl in 2% DMSO in OptiMEM) to achieve final concentrations between 0-30 μM. The cells were then returned to the incubator for a further 24 hours. 100 μl of luciferase reagent (Bright Glo, Promega) was added directly to each well, and the luminescence determined using a suitable luminometer.

To measure the selectivity of compounds, their ability to transactivate GAL4 fusions of PPARα LBD and PPARγ LBD was determined. The activity of compounds was expressed as a percentage relative to control compounds: PPARγ rosiglitazone (BRL 49653), PPARδ GW501516 (11) or PPARα KCL1999000269 (12). EC₅₀ values were calculated by fitting of the data to a sigmoidal dose response curve.

The compounds of the examples of the invention exhibited EC₅₀ values in the PPARδ GAL4 assay in the following categories as shown in tables 1 and 2 below: A represents an EC₅₀<0.1 μM; B represents an EC₅₀ in the range 0.1-1 μM; and C represents 1 P<EC₅₀≦30 μM.

TABLE 1

| Example | EC50 |
|---|---|
| 1 | A |
| 2 | A |
| 3 | B |
| 4 | B |
| 5 | B |
| 6 | C |
| 7 | C |
| 8 | B |
| 9 | B |
| 10 | B |
| 11 | C |
| 12 | B |
| 13 | C |
| 14 | C |
| 15 | C |
| 16 | C |
| 17 | C |
| 18 | C |
| 19 | B |
| 20 | C |
| 21 | C |
| 22 | A |
| 23 | B |
| 24 | B |
| 25 | C |
| 26 | C |
| 27 | B |
| 28 | A |
| 29 | A |
| 30 | C |
| 31 | B |
| 32 | C |
| 33 | B |
| 34 | B |
| 35 | C |
| 36 | A |
| 37 | B |
| 38 | A |
| 39 | A |
| 40 | A |
| 41 | C |
| 42 | C |
| 43 | B |
| 44 | B |
| 45 | A |
| 46 | C |
| 47 | B |
| 48 | C |
| 49 | C |
| 50 | C |
| 51 | C |
| 52 | C |
| 53 | C |
| 54 | B |
| 55 | C |
| 56 | C |
| 57 | C |
| 58 | B |
| 59 | B |
| 60 | C |
| 61 | C |
| 62 | C |
| 63 | C |
| 64 | C |
| 65 | C |
| 66 | C |
| 67 | A |
| 68 | C |
| 69 | C |
| 70 | C |
| 71 | B |
| 72 | B |
| 73 | C |
| 74 | C |
| 75 | A |
| 76 | A |
| 77 | A |
| 78 | C |
| 79 | A |
| 80 | A |
| 81 | B |
| 82 | C |
| 83 | A |
| 84 | B |
| 85 | B |
| 86 | B |
| 87 | B |
| 88 | C |
| 89 | C |
| 90 | C |
| 91 | C |
| 92 | B |
| 93 | B |
| 94 | B |
| 95 | B |
| 96 | B |
| 97 | B |
| 98 | B |
| 99 | A |
| 100 | B |
| 101 | A |
| 102 | B |
| 103 | B |

TABLE 2

| Example | EC50 |
|---|---|
| 104 | B |
| 105 | B |
| 106 | A |
| 107 | B |
| 108 | A |
| 109 | A |
| 110 | A |
| 111 | B |
| 112 | A |
| 113 | A |
| 114 | B |
| 115 | B |
| 116 | B |
| 117 | C |
| 118 | B |
| 119 | B |
| 120 | A |
| 121 | B |
| 122 | B |
| 123 | A |
| 124 | B |
| 125 | B |
| 126 | C |
| 127 | A |
| 128 | B |
| 129 | A |
| 130 | B |
| 131 | A |
| 132 | A |
| 133 | A |
| 134 | B |
| 135 | C |
| 136 | C |
| 137 | B |
| 138 | B |
| 139 | C |
| 140 | C |
| 141 | A |
| 142 | A |
| 143 | C |
| 144 | C |
| 145 | B |
| 146 | A |
| 147 | B |
| 148 | B |
| 149 | C |
| 150 | A |
| 151 | A |
| 152 | A |
| 153 | B |
| 154 | B |
| 155 | B |
| 156 | B |
| 157 | B |
| 158 | B |
| 159 | B |
| 160 | B |
| 161 | B |
| 162 | B |
| 163 | B |
| 164 | A |
| 165 | B |
| 166 | B |
| 167 | C |
| 168 | B |
| 169 | B |
| 170 | B |
| 171 | A |
| 172 | B |
| 173 | B |
| 174 | A |
| 175 | B |
| 176 | C |
| 177 | C |
| 178 | C |
| 179 | A |
| 180 | B |
| 181 | A |
| 182 | A |
| 183 | A |
| 184 | A |
| 185 | A |
| 186 | A |
| 187 | B |
| 188 | A |
| 189 | A |
| 190 | A |
| 191 | B |
| 192 | B |
| 193 | B |
| 194 | A |
| 195 | A |
| 196 | C |
| 197 | B |
| 198 | A |

### Biological Assay 2: Binding Assay

Compounds were tested for their ability to bind to PPARδ using a scintillation proximity assay (SPA). The PPARδ LBD (S139-Y441) was expressed in E. coli as an N-terminal GST fusion, with a hexhistidine tag immediately N-terminal to the PPARδ LBD. The purified protein was incubated with 3H GW2433 (for details of synthesis see reference 13) in the presence of varying concentrations of the compound to be tested in the presence of 5% DMSO. After 1 hour incubation at room temperature Yttrium silicate copper SPA bead were added and the incubation continued for a further 1 hour. After equilibration the radioactivity bound to the beads was determined by scintillation counting. Apparent Ki values were obtained by fitting the data by nonlinear regression analysis, assuming simple competitive binding. Non-specific binding was determined in the presence of excess unlabelled GW2433.

### Biological Assay 3: C2C12 Assay

C2C12 cells (ECACC, Salisbury, UK) were grown in Dulbecco's modified Eagle's medium supplemented with 200 units penicillin/50 μM streptomycin and 10% fetal calf serum. For cellular stimulation cells were seeded onto 6 cm dishes and grown until confluent. In order to induce differentiation the medium was changed to Dulbecco's modified Eagle's medium supplemented with 200 units penicillin/50 μM streptomycin and 2% horse serum. After 4 day of differentiation the cells were treated with the appropriate compound concentration (in a final of 0.1% DMSO) in the above mentioned medium for 24 h. Cells were lysed in 250 μl lysis solution and total RNA was extracted according to the manufacturer's protocol (Sigma Aldrich, St Louis, USA). cDNA was synthesized from 500 ng total RNA using random hexamers and multiscribe reverse transcriptase (Applied Biosystems) according to the manufacturer's protocol. Real time PCR was performed on the resulting cDNA using Applied Biosystems' Taqman method. In order to assess the beneficial effects of PPARδ agonists on β-oxidation and energy dissipation in muscle cells the following surrogate marker genes were analysed by real time quantitative PCR: FATP, LCAD, CPT1, PDK4, UCP2, UCP3, PGC-1a and GLUT4. Relative transcription levels were normalised to 18s ribosomal RNA levels.

### Biological Assay 4: In Vivo Study

In vivo studies were performed in ob/ob mice approximately 6 weeks old. Animals were fed for 14 days on a high fat diet and randomised by weight into groups. Compound or vehicle was administered daily by oral gavage for up to 4 weeks. The body weight and food intake was monitored daily and an oral glucose tolerance test performed periodically during the study. Blood samples were also taken for analysis to determine fasting levels of insulin, serum glucose, triglyceride, total and HDL-cholesterol and free fatty acids. Prior to termination all animals were subjected to DEXA scanning to assess body fat content. Following termination liver and muscle (gastrocnemius) tissue were excised from each animal for analysis of RNA.

Tissues were homogenised into Trizol solution (Invitrogen) and total RNA was extracted using a standard protocol. RNA was cleaned using the manufaturer's protocol (Sigma Aldrich, St Louis, USA). cDNA was synthesized from 500 ng total RNA using random hexamers and multiscribe reverse transcriptase (Applied Biosystems) according to the manufacturer's protocol. Real time PCR was performed on the resulting cDNA using Applied Biosystems' Taqman method. The following genes were analysed to determine whether favourable PPARδ-induced β-oxidation and energy uncoupling can be detected in the muscle samples: FATB, UCP2, UCP3, PGC1α, PDK4, CPT1, LCAD, GLUT4.

It will be understood that the invention is described above by way of example only and modifications may be made while remaining within the scope and spirit of the invention.

### REFERENCES

1 J. P. Berger et al., PPARs: therapeutic targets for metabolic disease, Trends Pharmacol Sci. (2005), 26(5), 244-251.
2 M. D. Leibowitz et al., Activation of PPARδ alters lipid metabolism in db/db mice, FEBS Lett. (2000), 473, 333-336.
3 W. R. Oliver et al., A selective peroxisome proliferator-activated receptor δ agonist promotes reverse cholesterol transport, Proc. Natl. Acad. Sci. USA (2001), 98, 5306-5311.
4 T. Tanaka et al., Activation of peroxisome proliferator-activated receptor delta induces fatty acid β-oxidation in skeletal muscle and attenuates metabolic syndrome, Proc. Natl. Acad. Sci. USA, (2003), 100, 15924-15929.
5 W.-X. Wang et al, Peroxisome-proliferator-activated receptor delta activates fat metabolism to prevent obesity, Cell (2003), 113, 159-170.
6 W.-X. Wang et al., Regulation of Muscle Fiber Type and Running Endurance by PPARδ, PLoS Biol. (2004), 2, 1532-1539.
7 Michalik et al., Impaired skin wound healing in peroxisome proliferator-activated receptor (PPAR)α and PPARβ mutant mice, J. Cell. Biol. (2001), 154, 799-819.
8 M. Schmuth et al., Peroxisome Proliferator-Activated Receptor (PPAR)-β/δ Stimulates Differentiation and Lipid Accumulation in Keratinocytes J. Invest. Dermatol. (2004), 122, 971-983.
9 S. J. Roberts-Thompson et al., Effect of the peroxisome proliferator-activated receptors activator GW0742 in rat cultured cerebellar granule neurons J. Neuroscience Research (2004), 77 (2), 240-249.
10 P. E. Polak et al., Protective effects of a peroxisome proliferator-activated receptor-β/δ agonist in experimental autoimmune encephalomyelitis, J. Neuroimmunol. (2005), In Press
11 M. Sznaidman et al., Novel Selective Small Molecule Agonists for Peroxisome Proliferator-Activated Receptor—Synthesis and Biological Activity, Biorg. Med. Chem. Lett. (2003), 13, 1517-1521.
12 M. Nomura et al., Design, Synthesis, and Evaluation of Substituted Phenylpropanoic Acid Derivatives as Human Peroxisome Proliferator Activated Receptor Activators. Discovery of Potent and Human Peroxisome Proliferator Activated Receptor αSubtype-Selective Activators, J. Med. Chem. (2003), 46, 3581.
13 P. Brown et al., Identification of peroxisome proliferator-activated receptor ligands from a biased chemical library, Chemistry & Biology (1997), 4, 909-918.

1. A compound of formula (I): 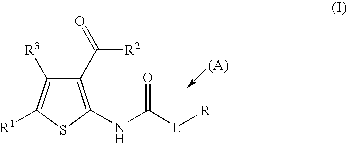
wherein:
R is a carboxylic acid or a derivative thereof;
R¹ is alkyl, alkenyl, alkynyl, cycloalkyl, alkoxy, alkylthio, halo or trihalomethyl;
R² is aryl, heteroaryl, arylalkyl or heteroarylalkyl;
R³ is H or F; and
L is a linking group comprising a chain of from 2 to 8 atoms linking R and the carbonyl group (A);
or a pharmaceutically acceptable derivative thereof.

2. A compound of claim 1 wherein R is a carboxylic acid.

3. A compound of claim 1 or claim 2 wherein R¹ is C₁₋₆alkyl, C₂₋₆alkenyl, C₂₋₆alkynyl, C₃₋₆cycloalkyl, C₁₋₆alkoxy, C₁₋₆alkylthio, halo or trihalomethyl.

4. A compound of claim 3 wherein R¹ is C₁₋₆alkyl or Cl.

5. A compound of any of claims 1-4 wherein R² is phenyl or pyridyl.

6. A compound of any of claims 1-5 wherein R³ is H.

7. A compound of any of claims 1-6 wherein L, in the orientation —(CO)-L-R₁ is -X-Y-Z-, where:
X is a single bond, alkylene, alkenylene, alkynylene, heteroalkylene, heteroalkenylene, heteroalkynylene, NR⁵, O, S, arylene, heteroarylene, cycloalkylene, heterocycloalkylene, cycloalkenylene or heterocycloalkenylene;
Y is a single bond, arylene, heteroarylene, cycloalkylene, heterocycloalkylene, cycloalkenylene or heterocycloalkenylene; and
Z is single bond, alkylene, alkenylene, alkynylene, heteroalkylene, heteroalkenylene, heteroalkynylene, NR⁵, O, S, arylene, heteroarylene, cycloalkylene, heterocycloalkylene, cycloalkenylene or heterocycloalkenylene; and
R⁵ is H, alkyl, aryl, —C(O)-alkyl, —C(O)-aryl, —S(O)₂-alkyl or —S(O)₂aryl;
provided that X, Y and Z are not each a single bond.

8. A compound of claim 7 wherein:
X is a single bond, alkylene, alkenylene, alkynylene, heteroalkylene, heteroalkenylene, heteroalkynylene, NR⁵, O, or S;
Y is a single bond, arylene, heteroarylene, cycloalkylene, heterocycloalkylene, cycloalkenylene or heterocycloalkenylene; and
Z is single bond, alkylene, alkenylene, alkynylene, heteroalkylene, heteroalkenylene, heteroalkynylene, NR⁵, O, or S;
provided that X, Y and Z are not each a single bond.

9. A compound of claim 7 or claim 8 wherein X is a single bond, alkylene, heteroalkylene, NR⁵ or O.

10. A compound of any of claims 7-9 wherein Y is a single bond, arylene, heteroarylene, cycloalkylene or heterocycloalkylene.

11. A compound of any of claims 7-10 wherein Z is a single bond, alkylene or heteroalkylene.

12. A compound of any of claims 1-8 wherein L is (in the orientation —(CO)-L-R)-(alkylene or heteroalkylene)-(arylene)-.

13. A compound of claim 12 wherein L is (in the orientation —(CO)-L-R) 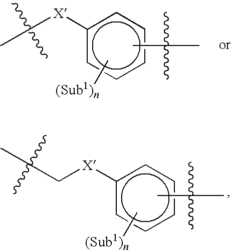
where:
X′ is CR⁷₂, O, S or NR⁶;
R⁶ is H, alkyl, aryl, —C(O)-alkyl, —C(O)-aryl, —S(O)₂-alkyl or —S(O)₂-aryl, or R⁶, together with a Sub¹ or R⁷ group, is alkylene;
R⁷ is independently H or Sub¹, or two R⁷ are alkylene or heteroalkylene;
n is 0, 1, 2 or 3;
Sub¹ is independently halogen, trihalomethyl, —NO₂, —CN, —N⁺(R^{s})₂O⁻, —CO₂H, —CO₂R₅, —SO₃H, —SOR^{s}, —SO₂R^{s}, —SO₃R^{s}, —OC(═O)ORS, —C(═O)H, —C(═O)R^{s}, —OC(═O)R^{s}, —NR^{s}₂, —C(═O)NH₂, —C(═O)NR^{s}₂, —N(R^{s})C(═O)OR^{s}, —N(R^{s})C(═O)NR^{s}₂, —OC(═O)NR^{s}₂, —N(R^{s})C(═O)R^{s}, —C(═S)NR^{s}₂, —NR^{s}C(═S)R^{s}, —SO₂NR^{s}₂, —NR^{s}SO₂R^{s}, —N(R^{s})C(═S)NR^{s}₂, —N(R^{s})SO₂NR^{s}₂, R^{s} or -Z^{s}R^{s};
Z^{s} is independently O, S or NR^{s};
R^{s} is independently H or C₁₋₆alkyl, C₃₋₆cycloalkyl, C₂₋₆alkenyl, C₃₋₆cycloalkenyl, C₃₋₆alkynyl, C₆₋₁₄aryl, heteroaryl having 5-13 members, C₆₋₁₄arylC₁₋₆alkyl, or heteroarylC₁₋₆alkyl where the heteroaryl has 5-13 members, where R^{s} is optionally substituted by 1 to 3 substituents Sub²;
Sub² is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z^{t}C₁₋₆alkyl; and
Z^{t} is O, S or N(C₁₋₆alkyl).

14. A compound of any of claims 1-8 wherein L is (in the orientation —(CO)-L-R), -(alkylene or heteroalkylene)-(arylene)-(alkylene or heteroalkylene)-.

15. A compound of claim 14 wherein L is (in the orientation —(CO)-L-R) 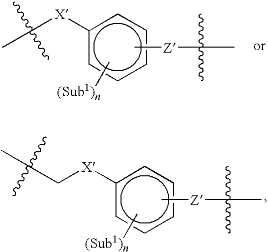
where:
Z′ is (in the orientation —(CO)— . . . -Z′-R)—CR⁷CR⁷—, —O—CR⁷—, —S—CR⁷— or —NR⁶—CR⁷—;
X′, R⁶, R⁷, Sub¹ and n are as defined in claim 13.

16. A compound of any of claims 1-8 wherein L is (in the orientation —(CO)-L-R)-(arylene)-(alkylene or heteroalkylene)-.

17. A compound of claim 16 wherein L is (in the orientation —(CO)-L-R) 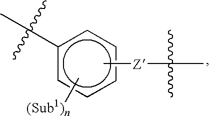
where:
Z′, Sub¹ and n are as defined in claim 13.

18. A compound of any of claims 1-8 wherein L is (in the orientation —CO)-L-R)-(alkylene or heteroalkylene)-.

19. A compound of claim 18 wherein L is (in the orientation —(CO)-L-R) 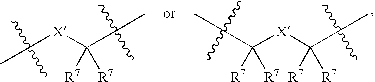
where:
X′ and R⁷ are as defined in claim 13.

20. A compound of any of claims 1-8 wherein L is (in the orientation —(CO)-L-R)-(arylene)-.

21. A compound of claim 20 wherein L is (in the orientation —(CO)-L-R) 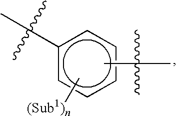
where:
Sub¹ and n are as defined in claim 13.

22. A compound of any of claims 1-21 wherein L comprises a chain of from 2 to 6 atoms linking R and the carbonyl group (A).

23. A compound of formula (II): 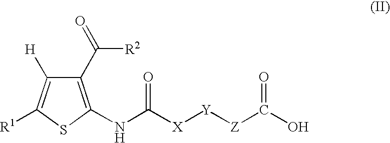
wherein:
R¹, R², X, Y and Z are as defined in any of claims 7-22;
or a pharmaceutically acceptable derivatives thereof.

24. A compound of claim 1 which is:
{[5-Ethyl-3-(4-trifluoromethoxy-benzoyl)-thiophen-2-ylcarbamoyl]-methylsulfanyl}-acetic acid
2-[5-Ethyl-3-(4-{-trifluoromethoxy-benzoyl)-thiophen-2-ylcarbamoyl]-methylsulfanyl}-2-methyl-propionic acid
4-[5-Ethyl-3-(4-trifluoromethoxy-benzoyl)-thiophen-2-ylcarbamoyl]-3,3-dimethyl-butyric acid
(1-{[5-Ethyl-3-(4-trifluoromethoxy-benzoyl)-thiophen-2-ylcarbamoyl]-methyl}-cyclopentyl)-acetic acid
2-{[5-Ethyl-3-(4-methoxy-benzoyl)-thiophen-2-ylcarbamoyl]-methylsulfanyl}-2-methyl-propionic acid
(1-{[5-Ethyl-3-(4-methoxy-benzoyl)-thiophen-2-ylcarbamoyl]-methyl}-cyclopentyl)-acetic acid
4-[5-Ethyl-3-(4-methoxy-benzoyl)-thiophen-2-ylcarbamoyl]-butylic acid
{[5-Ethyl-3-(4-methyl-benzoyl)-thiophen-2-ylcarbamoyl]-methylsulfanyl}-acetic acid
{[5-Ethyl-3-(4-ethyl-benzoyl)-thiophen-2-ylcarbamoyl]-methylsulfanyl}-acetic acid
2-{[5-Ethyl-3-(4-ethyl-benzoyl)-thiophen-2-ylcarbamoyl]-methylsulfanyl}-2-methyl-propionic acid
(1-{[5-Ethyl-3-(4-ethyl-benzoyl)-thiophen-2-ylcarbamoyl]-methyl}-cyclopentyl)-acetic acid
{[5-Ethyl-3-(4-phenoxy-benzoyl)-thiophen-2-ylcarbamoyl]-methylsulfanyl}-acetic acid
4-[5-Ethyl-3-(4-phenoxy-benzoyl)-thiophen-2-ylcarbamoyl]-3,3-dimethyl-butyric acid
[(3-Benzoyl-5-ethyl-thiophen-2-ylcarbamoyl)-methylsulfanyl]-acetic acid
2-[(3-Benzoyl-5-ethyl-thiophen-2-ylcarbamoyl)-methylsulfanyl]-2-methyl-propionic acid
4-(3-Benzoyl-5-ethyl-thiophen-2-ylcarbamoyl)-3,3-dimethyl-butyric acid
{1-[(3-Benzoyl-5-ethyl-thiophen-2-ylcarbamoyl)-methyl]-cyclopentyl}-acetic acid
{[5-Ethyl-3-(4-fluoro-benzoyl)-thiophen-2-ylcarbamoyl]-methylsulfanyl}-acetic acid
2-{[5-Ethyl-3-(4-fluoro-benzoyl)-thiophen-2-ylcarbamoyl]-methylsulfanyl}-2-methyl-propionic acid
4-[5-Ethyl-3-(4-fluoro-benzoyl)-thiophen-2-ylcarbamoyl]-3,3-di-ethyl-butyric acid
(1-{[5-Ethyl-3-(4-fluoro-benzoyl)-thiophen-2-ylcarbamoyl]-methyl}-cyclopentyl)-acetic acid
{[3-(4-Bromo-benzoyl)-5-ethyl-thiophen-2-ylcarbamoyl]-methylsulfanyl}-acetic acid
{[3-(4-Chloro-benzoyl)-5-ethyl-thiophen-2-ylcarbamoyl]-methylsulfanyl}-acetic acid
2-{[3-(4-Chloro-benzoyl)-5-ethyl-thiophen-2-ylcarbamoyl]-methylsulfanyl}-2-methyl-propionic acid
4-[3-(4-Chloro-benzoyl)-5-ethyl-thiophen-2-ylcarbamoyl]-3,3-dimethyl-butyric acid
(1-{[3-(4-Chloro-benzoyl)-5-ethyl-thiophen-2-ylcarbamoyl]-methyl}-cyclopentyl)-acetic acid
{[3-(3-Chloro-benzoyl)-5-ethyl-thiophen-2-ylcarbamoyl]-methylsulfanyl}-acetic acid
{[3-(3,4-Dichloro-benzoyl)-5-ethyl-thiophen-2-ylcarbamoyl]-methylsulfanyl}-acetic acid
2-{[3-(3,4-Dichloro-benzoyl)-5-ethyl-thiophen-2-ylcarbamoyl]-methylsulfanyl}-2-methyl-propionic acid
4-[3-(3,4-Dichloro-benzoyl)-5-ethyl-thiophen-2-ylcarbamoyl]-3,3-dimethyl-butyric acid
{[3-(3-Chloro-4-fluoro-benzoyl)-5-ethyl-thiophen-2-ylcarbamoyl]-methylsulfanyl}-acetic acid
4-[3-(3-Chloro-4-fluoro-benzoyl)-5-ethyl-thiophen-2-ylcarbamoyl]-3,3-dimethyl-butyric acid
{[5-Ethyl-3-(4-isopropoxy-benzoyl)-thiophen-2-ylcarbamoyl]-methylsulfanyl}-acetic acid
{[3-(3-Bromo-benzoyl)-5-ethyl-thiophen-2-ylcarbamoyl]-methylsulfanyl}-acetic acid
{[3-(4-Cyano-benzoyl)-5-ethyl-thiophen-2-ylcarbamoyl]-methylsulfanyl}-acetic acid
{[3-(Biphenyl-4-carbonyl)-5-ethyl-thiophen-2-ylcarbamoyl]-methylsulfanyl}-acetic acid
4-[3-(Biphenyl-4-carbonyl)-5-ethyl-thiophen-2-ylcarbamoyl]-3,3-dimethyl-butyric acid
{[5-Ethyl-3-(4′-trifluoromethyl-biphenyl-4-carbonyl)-thiophen-2-ylcarbamoyl]-methylsulfanyl}-acetic acid
{[5-Ethyl-3-(4′-trifluoromethoxy-biphenyl-4-carbonyl)-thiophen-2-ylcarbamoyl]-methylsulfanyl}-acetic acid
{[5-Ethyl-3-(4′-fluoro-biphenyl-4-carbonyl)-thiophen-2-ylcarbamoyl]-methylsulfanyl}-acetic acid
{[5-Ethyl-3-(4-pyridin-5-yl-benzoyl)-thiophen-2-ylcarbamoyl]-methylsulfanyl}-acetic acid
({5-Ethyl-3-[4-(1-methyl-1H-pyrazol-4-yl)-benzoyl]-thiophen-2-ylcarbamoyl}-methylsulfanyl)-acetic acid
{[3-(3-Bromo-benzoyl)-5-ethyl-thiophen-2-ylcarbamoyl]-methylsulfanyl}-acetic acid
{[5-Ethyl-3-(4-trifluoromethoxy-biphenyl-3-carbonyl)-thiophen-2-ylcarbamoyl]-methylsulfanyl}-acetic acid
{[5-Ethyl-3-(4-trifluoromethylbenzoyl)-thiophen-2-ylcarbamoyl]-methylsulfanyl}-acetic acid
{[5-Ethyl-3-(naphthalene-1-carbonyl)-thiophen-2-ylcarbamoyl]-methylsulfanyl}-acetic acid
{[5-Ethyl-3-(naphthalene-2-carbonyl)-thiophen-2-ylcarbamoyl]-methylsulfanyl}-acetic acid
{[5-Ethyl-3-(3-methoxybenzoyl)-thiophen-2-ylcarbamoyl]-methylsulfanyl}-acetic acid
{[3-(3,4-Dimethoxybenzoyl)-5-ethyl-thiophen-2-ylcarbamoyl]-methylsulfanyl}-acetic acid
{[3-(4-tert-Butyl-benzoyl)-5-ethyl-thiophen-2-ylcarbamoyl]-methylsulfanyl}-acetic acid
{[3-(3,4-Dimethyl-benzoyl)-5-ethyl-thiophen-2-ylcarbamoyl]-methylsulfanyl}-acetic acid
4-[3-(3,4-Dimethyl-benzoyl)-5-ethyl-thiophen-2-ylcarbamoyl]-3,3-dimethyl-butyric acid
(1-{[3-(3,4-Dimethyl-benzoyl)-5-ethyl-thiophen-2-ylcarbamoyl]-methyl}-cyclopentyl)-acetic acid
2-{[3-(3,4-Dimethyl benzoyl)-5-ethyl-thiophen-2-ylcarbamoyl]-methylsulfanyl}-2-methyl-propionic acid
{[5-Ethyl-3-(6-methoxy-pyridine-3-carbonyl)-thiophen-2-ylcarbamoyl]-methylsulfanyl}-acetic acid
(1-{[5-Ethyl-3-(6-methoxy-pyridine-3-carbonyl)-thiophen-2-ylcarbamoyl]-methyl}-cyclopentyl)-acetic acid
{[5-Ethyl-3-(6-trifluoromethyl-pyridine-3-carbonyl)-thiophen-2-ylcarbamoyl]-methylsulfanyl}-acetic acid
{[5-Ethyl-3-(6-isopropoxy-pyridine-3-carbonyl)-thiophen-2-ylcarbamoyl]-methylsulfanyl}-acetic acid
{[3-(5-Chloro-6-isopropoxy-pyridine-3-carbonyl)-5-ethyl-thiophen-2-ylcarbamoyl]-methylsulfanyl}-acetic acid
{[5-Ethyl-3-(6-phenoxy-pyridine-3-carbonyl)-thiophen-2-ylcarbamoyl]-methylsulfanyl}-acetic acid
{[3-(4-Methoxy-benzoyl)-5-methyl-thiophen-2-ylcarbamoyl]-methylsulfanyl}-acetic acid
{[5-Isopropyl-3-(4-methoxy-benzoyl)-thiophen-2-ylcarbamoyl]-methylsulfanyl}-acetic acid
{[3-(4-Methoxybenzoyl)-5-propyl-thiophen-2-ylcarbamoyl]-methylsulfanyl}-acetic acid
{[5-Cyclopropyl-3-(4-fluoro-benzoyl)-thiophen-2-ylcarbamoyl]-methylsulfanyl}-acetic acid
{[5-Chloro-3-(6-methoxy-pyridine-3-carbonyl)-thiophen-2-ylcarbamoyl]-methylsulfanyl}-acetic acid
{[5-Chloro-3-(4-methoxy-benzoyl)-thiophen-2-ylcarbamoyl]-methylsulfanyl}-acetic acid
{[5-Chloro-3-(4-trifluoromethoxy-benzoyl)-thiophen-2-ylcarbamoyl]-methylsulfanyl}-acetic acid
{[3-(4-Methoxy-benzoyl)-thiophen-2-ylcarbamoyl]-methylsulfanyl}-acetic acid
5-[5-Ethyl-3-(4-methoxy-benzoyl)-thiophen-2-ylcarbamoyl]-pentanoic acid
6-[5-Ethyl-3-(4-methoxy-benzoyl)-thiophen-2-ylcarbamoyl]-hexanoic acid
{3-[5-Ethyl-3-(4-methoxy-benzoyl)-thiophen-2-ylcarbamoyl]-propylsulfanyl}-acetic acid
{1-[5-Ethyl-3-(4-methoxy-benzoyl)-thiophen-2-ylcarbamoyl]-ethylsulfanyl}-acetic acid
({[5-Ethyl-3-(4-methoxy-benzoyl)-thiophen-2-ylcarbamoyl]-methyl}-methyl-amino)-acetic acid hydrochloride
({[5-Ethyl-3-(4-methoxy-benzoyl)-thiophen-2-ylcarbamoyl]-methyl}-amino)-acetic acid hydrochloride
2-({[5-Ethyl-3-(4-trifluoromethoxy-benzoyl)-thiophen-2-ylcarbamoyl]-methyl}-amino)-3-methyl-butyric acid
2-({[5-Ethyl-3-(4-trifluoromethoxy-benzoyl)-thiophen-2-ylcarbamoyl]-methyl}-amino)-2-methyl-propionic acid
2-({[5-Ethyl-3-(4-trifluoromethoxy-benzoyl)-thiophen-2-ylcarbamoyl]-methyl}-amino)-4-methyl-pentanoic acid
(R)-2-({[5-Ethyl-3-(4-trifluoromethoxy-benzoyl)-thiophen-2-ylcarbamoyl]-methyl}-amino)-3-methyl-butyric acid
(S)-2-({[5-Ethyl-3-(4-trifluoromethoxy-benzoyl)-thiophen-2-ylcarbamoyl]-methyl}-amino)-3-methyl-butyric acid
2-({[5-Ethyl-3-(4-trifluoromethoxy-benzoyl)-thiophen-2-ylcarbamoyl]-methyl}-amino)-butyric acid
({[5-Ethyl-3-(4-trifluoromethoxy-benzoyl)-thiophen-2-ylcarbamoyl]-methyl}-amino)-acetic acid
1-({[5-Ethyl-3-(4-trifluoromethoxy-benzoyl)-thiophen-2-ylcarbamoyl]-methyl}-amino)-cyclopropanecarboxylic acid
2-({[5-Ethyl-3-(4′-fluoro-biphenyl-4-carbonyl)-thiophen-2-ylcarbamoyl]-methyl}-amino)-2-methyl-propionic acid
2-({[5-Ethyl-3-(4′-trifluoromethoxy-biphenyl-3-carbonyl)-thiophen-2-yl carbamoyl]-methyl}-amino)-2-methyl-propionic acid
2-({[5-Ethyl-3-(4′-fluoro-biphenyl-3-carbonyl)-thiophen-2-ylcarbamoyl]-methyl}-amino)-2-methyl-propionic acid
2-({[5-Chloro-3-(4-trifluoromethoxy-benzoyl)-thiophen-2-ylcarbamoyl]-methyl}-amino)-2-methyl-propionic acid
({1-[3-(4-Chloro-benzoyl)-5-ethyl-thiophen-2-ylcarbamoyl]-ethyl}-methyl-amino)-acetic acid
(3-{[5-Ethyl-3-(4-methoxy-benzoyl)-thiophen-2-ylcarbamoyl]-methylsulfanyl}-phenoxy)-acetic acid
(4-{[5-Ethyl-3-(4-methoxy-benzoyl)-thiophen-2-ylcarbamoyl]-methylsulfanyl}-phenoxy)-acetic acid
3-{[5-Ethyl-3-(4-methoxy-benzoyl)-thiophen-2-ylcarbamoyl]-methylsulfanyl}-benzoic acid
(4-{[5-Ethyl-3-(4-methoxy-benzoyl)-thiophen-2-ylcarbamoyl]-methylsulfanyl}-2-methyl-phenoxy)-acetic acid
2-(4-{[5-Ethyl-3-(4-methoxy-benzoyl)-thiophen-2-ylcarbamoyl]-methylsulfanyl}-phenoxy)-2-methyl-propionic acid
(4-{[5-Ethyl-3-(4-trifluoromethoxy-benzoyl)-thiophen-2-ylcarbamoyl]-methylsulfanyl}-phenoxy)-acetic acid
(4-{[5-Ethyl-3-(4-trifluoromethoxy-benzoyl)-thiophen-2-ylcarbamoyl]-methylsulfanyl}-2-methyl-phenoxy)-acetic acid
2-(4-{[5-Ethyl-3-(4-trifluoromethoxy-benzoyl)-thiophen-2-ylcarbamoyl]-methylsulfanyl}-phenoxy)-2-methyl-propionic acid
3-(4-{[5-Ethyl-3-(4-trifluoromethoxy-benzoyl)-thiophen-2-ylcarbamoyl]-methylsulfanyl}-phenyl)-propionic acid
{4-[3-(4-Chloro-benzoyl)-5-ethyl-thiophen-2-ylcarbamoyl]-phenoxy}-acetic acid
{3-[5-Ethyl-3-(4-trifluoromethoxy-benzoyl)-thiophen-2-ylcarbamoyl]-phenylsulfanyl}-acetic acid
{4-[5-Ethyl-3-(4-trifluoromethoxy-benzoyl)-thiophen-2-ylcarbamoyl]-phenoxy}-acetic acid
{3-[5-Ethyl-3-(4-trifluoromethoxy-benzoyl)-thiophen-2-ylcarbamoyl]-phenoxy}-acetic acid
{4-[5-Ethyl-3-(4-trifluoromethoxy-benzoyl)-thiophen-2-ylcarbamoyl]-2-methyl-phenoxy}-acetic acid
2-{4-[5-Ethyl-3-(4-trifluoromethoxy-benzoyl)-thiophen-2-ylcarbamoyl]-phenoxy}-2-methyl-propionic acid or
{4-[5-Ethyl-3-(4-trifluoromethoxy-benzoyl)-thiophen-2-ylcarbamoyl]-phenylsulfanyl}-acetic acid.

25. A compound of claim 1 which is:
4-[5-Ethyl-3-(4-trifluoromethoxy-benzoyl)-thiophen-2-ylcarbamoyl]-butyric acid
{3-[5-Ethyl-3-(4-trifluoromethoxy-benzoyl)-thiophen-2-ylcarbamoyl]-propylsulfanyl}-acetic acid
{1-[5-Ethyl-3-(4-trifluoromethoxy-benzoyl)-thiophen-2-ylcarbamoyl]-ethylsulfanyl}-acetic acid
{1-[5-Ethyl-3-(4-trifluoromethoxy-benzoyl)-thiophen-2-ylcarbamoyl]-1-methyl-ethylsulfanyl}-acetic acid
2-{1-[5-Ethyl-3-(4-trifluoromethoxy-benzoyl)-thiophen-2-ylcarbamoyl]-1-methyl-ethylsulfanyl}-propionic acid
{1-[5-Ethyl-3-(4-trifluoromethoxy-benzoyl)-thiophen-2-ylcarbamoyl]-propylsulfanyl}-acetic acid
2-{1-[5-Ethyl-3-(4-trifluoromethoxy-benzoyl)-thiophen-2-ylcarbamoyl]-propylsulfanyl}-propionic acid
2-{[5-Ethyl-3-(4-methoxy-benzoyl)-thiophen-2-ylcarbamoyl]-methylsulfanyl}-propionic acid
2-{[5-Ethyl-3-(4-trifluoromethoxy-benzoyl)-thiophen-2-ylcarbamoyl]-methylsulfanyl}-propionic acid
{[5-Ethyl-3-(4-trifluoromethoxy-benzoyl)-thiophen-2-ylcarbamoyl]-phenyl-methylsulfanyl}-acetic acid
{1-[5-Ethyl-3-(4-trifluoromethoxy-benzoyl)-thiophen-2-ylcarbamoyl]-2-methyl-propylsulfanyl}-acetic acid
{4-[5-Ethyl-3-(4-trifluoromethoxy-benzoyl)-thiophen-2-ylcarbamoyl]-phenylamino}-acetic acid
3-{4-[5-Ethyl-3-(4-trifluoromethoxy-benzoyl)-thiophen-2-ylcarbamoyl]-phenyl}-propionic acid
(4-{[5-Ethyl-3-(4-trifluoromethoxy-benzoyl)-thiophen-2-ylcarbamoyl]-methoxy}-phenoxy)-acetic acid
{1-[5-Ethyl-3-(4-trifluoromethoxy-benzoyl)-thiophen-2-ylcarbamoyl]-1-methyl-ethylamino}-acetic acid
(R)-1-{1-[5-Ethyl-3-(4-trifluoromethoxy-benzoyl)-thiophen-2-ylcarbamoyl]-1-methyl-ethyl}-pyrrolidine-2-carboxylic acid
(R)-1-{1-[5-Ethyl-3-(4-trifluoromethoxy-benzoyl)-thiophen-2-ylcarbamoyl]-propyl}-pyrrolidine-2-carboxylic acid
{1-[5-Ethyl-3-(4-trifluoromethoxy-benzoyl)-thiophen-2-ylcarbamoyl]-propylamino}-acetic acid
({[5-Ethyl-3-(4-trifluoromethoxy-benzoyl)-thiophen-2-ylcarbamoyl]-phenyl-methyl}-amino)-acetic acid
(R)-1-{1-[5-Ethyl-3-(4-trifluoromethoxy-benzoyl)-thiophen-2-ylcarbamoyl]-ethyl}-pyrrolidine-2-carboxylic acid
(R)-1-{[5-Ethyl-3-(4-trifluoromethoxy-benzoyl)-thiophen-2-ylcarbamoyl]-phenyl-methyl}-pyrrolidine-2-carboxylic acid
(S)-2-(Ethyl-{[5-ethyl-3-(4-trifluoromethoxy-benzoyl)-thiophen-2-ylcarbamoyl]-methyl}-amino)-propionic acid
(S)-2-({[5-Ethyl-3-(4-trifluoromethoxy-benzoyl)-thiophen-2-ylcarbamoyl]-methyl}-methanesulfonyl-amino)-propionic acid
2-{[5-Ethyl-3-(4-trifluoromethoxy-benzoyl)-thiophen-2-ylcarbamoyl]-methylsulfanyl}-butyric acid
{[5-Ethyl-3-(4-trifluoromethoxy-benzoyl)-thiophen-2-ylcarbamoyl]-methylsulfanyl}-phenyl-acetic acid
(S)-2-{[5-Ethyl-3-(4-trifluoromethoxy-benzoyl)-thiophen-2-ylcarbamoyl]-methylsulfanyl}-propionic acid
{[3-(3-Chloro-4-isopropoxy-benzoyl)-5-ethyl-thiophen-2-ylcarbamoyl]-methylsulfanyl}-acetic acid
{[5-Ethyl-3-(3-fluoro-4-trifluoromethoxy-benzoyl)-thiophen-2-ylcarbamoyl]-methylsulfanyl}-acetic acid
{[5-Propyl-3-(4-trifluoromethoxy-benzoyl)-thiophen-2-ylcarbamoyl]-methylsulfanyl}-acetic acid
{[5-Isopropyl-3-(4-trifluoromethoxy-benzoyl)-thiophen-2-ylcarbamoyl]-methylsulfanyl}-acetic acid
{[5-sec-Butyl-3-(4-trifluoromethoxy-benzoyl)-thiophen-2-ylcarbamoyl]-methylsulfanyl}-acetic acid
(4-{[5-Ethyl-3-(4-fluoro-benzoyl)-thiophen-2-ylcarbamoyl]-methylsulfanyl}-phenoxy)-acetic acid
(4-{[3-(4-Chloro-benzoyl)-5-ethyl-thiophen-2-ylcarbamoyl]-methylsulfanyl}-phenoxy)-acetic acid
2-(4-{[5-Ethyl-3-(4-fluoro-benzoyl)-thiophen-2-ylcarbamoyl]-methylsulfanyl}-phenoxy)-2-methyl-propionic acid
2-(4-{[3-(4-Chloro-benzoyl)-5-ethyl-thiophen-2-ylcarbamoyl]-methylsulfanyl}-phenoxy)-2-methyl-propionic acid
{[3-(Benzothiazole-2-carbonyl)-5-ethyl-thiophen-2-ylcarbamoyl]-methylsulfanyl}-acetic acid
{[3-(Benzofuran-2-carbonyl)-5-ethyl-thiophen-2-ylcarbamoyl]-methylsulfanyl}-acetic acid
(4-{[5-Methyl-3-(4-trifluoromethoxy-benzoyl)-thiophen-2-ylcarbamoyl]-methylsulfanyl}-phenoxy)-acetic acid
{[3-(3-Chloro-4-trifluoromethoxy-benzoyl)-5-ethyl-thiophen-2-ylcarbamoyl]-methylsulfanyl}-acetic acid
{[5-Ethyl-3-(3-trifluoromethoxy-benzoyl)-thiophen-2-ylcarbamoyl]-methylsulfanyl}-acetic acid
{[3-(1,5-Dimethyl-1H-pyrazole-3-carbonyl)-5-ethyl-thiophen-2-ylcarbamoyl]-methylsulfanyl}-acetic acid
{[5-Ethyl-3-(4-pyridin-2-yl-benzoyl)-thiophen-2-ylcarbamoyl]-methylsulfanyl}-acetic acid
4-[5-Ethyl-3-(4-trifluoromethoxy-benzoyl)-thiophen-2-ylcarbamoyl]-2,2-di-ethyl-butyric acid
4-[3-(4-Chloro-benzoyl)-5-ethyl-thiophen-2-ylcarbamoyl]-2,2-dimethyl-butyric acid
4-[5-Ethyl-3-(3-fluoro-4-trifluoromethoxy-benzoyl)-thiophen-2-ylcarbamoyl]-2,2-dimethyl-butyric acid
{[5-Ethyl-3-(1-methyl-1H-indole-2-carbonyl)-thiophen-2-ylcarbamoyl]-methylsulfanyl}-acetic acid
{[5-Ethyl-3-(1-methyl-5-trifluoromethoxy-1H-indole-2-carbonyl)-thiophen-2-ylcarbamoyl]-methylsulfanyl}-acetic acid
{[3-(5-Chloro-1-methyl-1H-indole-2-carbonyl)-5-ethyl-thiophen-2-ylcarbamoyl]-methylsulfanyl}-acetic acid
4-[5-Ethyl-3-(1-methyl-5-trifluoromethoxy-H-indole-2-carbonyl)-thiophen-2-ylcarbamoyl]-2,2-dimethyl-butyric acid
{[5-Ethyl-3-(6-trifluoroethoxy-benzothiazole-2-carbonyl)-thiophen-2-ylcarbamoyl]-methylsulfanyl}-acetic acid
{[3-(6-Chloro-benzothiazole-2-carbonyl)-5-ethyl-thiophen-2-ylcarbamoyl]-methylsulfanyl}-acetic acid
{[3-(5-Chloro-benzothiazole-2-carbonyl)-5-ethyl-thiophen-2-ylcarbamoyl]-methylsulfanyl}-acetic acid
{[3-(6-Chloro-quinoline-2-carbonyl)-5-ethyl-thiophen-2-ylcarbamoyl]-methylsulfanyl}-acetic acid
{[3-(5-Chloro-1-methyl-1H-indole-3-carbonyl)-5-ethyl-thiophen-2-ylcarbamoyl]-methylsulfanyl}-acetic acid
{1-[5-Ethyl-3-(4-trifluoromethoxy-benzoyl)-thiophen-2-ylcarbamoyl]-ethylamino}-acetic acid
1-{[5-Ethyl-3-(4-trifluoromethoxy-benzoyl)-thiophen-2-ylcarbamoyl]-methyl}-piperidine-3-carboxylic acid
(S)-2-({[5-Ethyl-3-(4-trifluoromethoxy-benzoyl)-thiophen-2-ylcarbamoyl]-methyl}-amino)-propionic acid
(R)-2-({[5-Ethyl-3-(4-trifluoromethoxy-benzoyl)-thiophen-2-ylcarbamoyl]-methyl}-amino)-propionic acid
1-{[5-Ethyl-3-(4-trifluoromethoxy-benzoyl)-thiophen-2-ylcarbamoyl]-methyl}-pyrrolidine-3-carboxylic acid
1-{[5-Ethyl-3-(4-trifluoromethoxy-benzoyl)-thiophen-2-ylcarbamoyl]-methyl}-piperidine-4-carboxylic acid
1-({[5-Ethyl-3-(4-trifluoromethoxy-benzoyl)-thiophen-2-ylcarbamoyl]-methyl}-amino)-cyclobutanecarboxylic acid
1-{[5-Ethyl-3-(4-trifluoromethoxy-benzoyl)-thiophen-2-ylcarbamoyl]-methyl}-piperidine-2-carboxylic acid
1-({[5-Ethyl-3-(4-trifluoromethoxy-benzoyl)-thiophen-2-ylcarbamoyl]-methyl}-amino)-cyclohexanecarboxylic acid
(S)-1-{[5-Ethyl-3-(4-trifluoromethoxy-benzoyl)-thiophen-2-ylcarbamoyl]-methyl}-pyrrolidine-2-carboxylic acid
(R)-1-{[5-Ethyl-3-(4-trifluoromethoxy-benzoyl)-thiophen-2-ylcarbamoyl]-methyl}-pyrrolidine-2-carboxylic acid
(S)-2-({[3-(3-Chloro-4-trifluoromethoxy-benzoyl)-5-ethyl-thiophen-2-ylcarbamoyl]-methyl}-amino)-3-methyl-butyric acid
(S)-2-({[3-(3,4-Dichloro-benzoyl)-5-ethyl-thiophen-2-ylcarbamoyl]-methyl}-amino)-3-methyl-butyric acid
2-({[3-(3-Chloro-4-trifluoromethoxy-benzoyl)-5-ethyl-thiophen-2-ylcarbamoyl]-methyl}-amino)-2-methyl-propionic acid
(R)-1-{[5-Ethyl-3-(4-trifluoromethoxy-benzoyl)-thiophen-2-ylcarbamoyl]-methyl}-2-methyl-pyrrolidine-2-carboxylic acid
1-{[5-Ethyl-3-(4-trifluoromethoxy-benzoyl)-thiophen-2-ylcarbamoyl]-methyl}-2-methyl-pyrrolidine-2-carboxylic acid
4-[5-Ethyl-3-(4-trifluoromethoxy-benzoyl)-thiophen-2-ylcarbamoyl]-3-methyl-butyric acid
({5-Ethyl-3-[4-(2,2,2-trifluoro-ethoxy)-benzoyl]-thiophen-2-ylcarbamoyl}-methylsulfanyl)-acetic acid
{3-[5-Ethyl-3-(4-trifluoromethoxy-benzoyl)-thiophen-2-ylcarbamoyl]-piperidin-1-yl}-acetic acid
{4-[5-Ethyl-3-(4-trifluoromethoxy-benzoyl)-thiophen-2-ylcarbamoyl]-piperidin-1-yl}-acetic acid
(2R*,5R*)-1-{[5-Ethyl-3-(4-trifluoromethoxy-benzoyl)-thiophen-2-ylcarbamoyl]-methyl}-5-methyl-pyrrolidine-2-carboxylic acid
(2R*,5S*)-1-{[5-Ethyl-3-(4-trifluoromethoxy-benzoyl)-thiophen-2-ylcarbamoyl]-methyl}-5-methyl-pyrrolidine-2-carboxylic acid
1-{[5-Ethyl-3-(4-trifluoromethoxy-benzoyl)-thiophen-2-ylcarbamoyl]-methyl}-4-methyl-pyrrolidine-2-carboxylic acid
(2R,5R)-1-{[5-Ethyl-3-(4-trifluoromethoxy-benzoyl)-thiophen-2-ylcarbamoyl]-methyl}-5-(4-fluoro-phenyl)-pyrrolidine-2-carboxylic acid
(2R,5S)-1-{[5-Ethyl-3-(4-trifluoromethoxy-benzoyl)-thiophen-2-ylcarbamoyl]-methyl}-5-methyl-pyrrolidine-2-carboxylic acid
(2R,5S)-5-Ethyl-1-{[5-ethyl-3-(4-trifluoromethoxy-benzoyl)-thiophen-2-ylcarbamoyl]-methyl}-pyrrolidine-2-carboxylic acid
(2R,5R)-1-{[5-Ethyl-3-(4-trifluoromethoxy-benzoyl)-thiophen-2-ylcarbamoyl]-ethyl}-5-phenyl-pyrrolidine-2-carboxylic acid
(2R,5R)-1-{[5-Ethyl-3-(4-trifluoromethoxy-benzoyl)-thiophen-2-ylcarbamoyl]-methyl}-5-isopropyl-pyrrolidine-2-carboxylic acid
(2R,5S)-1-{1-[5-Ethyl-3-(4-trifluoromethoxy-benzoyl)-thiophen-2-ylcarbamoyl]-ethyl}-5-methyl-pyrrolidine-2-carboxylic acid
(2R,5R)-1-{[3-(4-Chloro-benzoyl)-5-ethyl-thiophen-2-ylcarbamoyl]-methyl}-5-phenyl-pyrrolidine-2-carboxylic acid hydrochloride
(2R,5R)-1-{[5-Chloro-3-(4-trifluoromethoxy-benzoyl)-thiophen-2-ylcarbamoyl]-methyl}-5-phenyl-pyrrolidine-2-carboxylic acid hydrochloride
1-{[5-Ethyl-3-(4-trifluoromethoxy-benzoyl)-thiophen-2-ylcarbamoyl]-methyl}-pyrrolidine-2-carboxylic acid
1-({[5-Ethyl-3-(4-trifluoromethoxy-benzoyl)-thiophen-2-ylcarbamoyl]-methyl}-amino)-cyclopentanecarboxylic acid
(S)-2-({[5-Ethyl-3-(4-trifluoromethoxy-benzoyl)-thiophen-2-ylcarbamoyl]-methyl}-amino)-butyric acid
(S)-2-({[5-Ethyl-3-(4-trifluoromethoxy-benzoyl)-thiophen-2-ylcarbamoyl]-methyl}-amino)-2-methyl-butyric acid
(R)-1-{2-[5-Ethyl-3-(4-trifluoromethoxy-benzoyl)-thiophen-2-ylcarbamoyl]-ethyl}-pyrrolidine-2-carboxylic acid
{2-[5-Ethyl-3-(4-trifluoromethoxy-benzoyl)-thiophen-2-ylcarbamoyl]-ethylsulfanyl}-acetic acid
{(S)-1-[5-Ethyl-3-(4-trifluoromethoxy-benzoyl)-thiophen-2-ylcarbamoyl]-ethylsulfanyl}-acetic acid
2-({[5-Ethyl-3-(4-trifluoromethoxy-benzoyl)-thiophen-2-ylcarbamoyl]-methyl}-methyl-amino)-2-methyl-propionic acid
1-({[5-Ethyl-3-(4-trifluoromethoxy-benzoyl)-thiophen-2-ylcarbamoyl]-methyl}-methyl-amino)-cyclopropanecarboxylic acid or
(S)-2-({[5-Ethyl-3-(4-trifluoromethoxy-benzoyl)-thiophen-2-ylcarbamoyl]-methyl}-methyl-amino)-propionic acid

26. A compound of any of claims 1-25 for use in therapy.

27. A pharmaceutical composition comprising a compound of any of claims 1-25 in combination with a pharmaceutically acceptable carrier, excipient or diluent.

28. A method for the treatment of a disease or condition mediated by PPARδ, comprising the step of administering a therapeutically effective amount of a compound of any of claims 1-25 to a patient.

29. The use of a compound of any of claims 1-25 in the manufacture of a medicament for the treatment of a disease or condition mediated by PPARδ.

30. The method of claim 28 or the use of claim 29 wherein the disease or condition is: metabolic syndrome, or a component thereof, e.g. dyslipidaemia, obesity or insulin resistance; type-II diabetes; wound healing; inflammation; a neurodegenerative disorder; or multiple sclerosis.

31. The method of claim 28 or the use of claim 29 wherein the disease or condition is: coronary heart disease; hypertension; hyperlipidaemia; type-II diabetes mellitus; stroke; osteoarthritis; restrictive pulmonary disease; sleep apnoea or cancer.

32. A crystal of PPARδ and a compound of any of claims 1-25.
